(19) United States
(12) Patent Application Publication
(10) Pub. No.: US 2017/0342046 A1
(43) Pub. Date: Nov. 30, 2017

(54) Fatty Acid Cysteine-Based Conjugates and Their Use in Treating Medical Disorders

(71) Applicant: Catabasis Pharmaceuticals, Inc., Cambridge, MA (US)

(72) Inventor: Chi B. Vu, Boston, MA (US)

(21) Appl. No.: 15/604,000

(22) Filed: May 24, 2017

Related U.S. Application Data
(60) Provisional application No. 62/340,792, filed on May 24, 2016.

(51) Int. Cl. C07D 317/60; C07C 323/60; C07C 323/41; C07C 323/59; C07D 261/18; C07D 213/82

(52) U.S. Cl. CPC C07D 317/60; C07D 261/18; C07D 213/82; C07C 323/41; C07C 323/59; C07C 323/60

(57) ABSTRACT

The invention relates to fatty acid cysteine-based conjugates, compositions comprising a fatty acid cysteine-based conjugates, and methods for using such conjugates and compositions to treat disease, such as a disease caused by dysregulation of autophagy or mitochondrial bioenergetics.

### CROSS-REFERENCE TO RELATED APPLICATIONS

This application claims priority to and the benefit of U.S. Provisional Patent Application No. 62/340,792, filed May 24, 2016, the contents of which are incorporated by reference herein in their entirety.

### FIELD OF THE INVENTION

The invention relates to fatty acid cysteine-based conjugates, compositions comprising a fatty acid cysteine conjugate, and methods for using such conjugates and compositions to treat disease such as a disease caused by dysregulation of autophagy or mitochondrial bioenergetics.

### BACKGROUND

Autophagy is an evolutionarily conserved lysosomal degradation pathway to essentially self-digest some cellular components (see, Levine and Kroemer (2008) CELL, 132, p. 27-42). This self-digestion process helps cells remove extraneous or damaged organelles, defective or mis-folded proteins, and even invading microorganisms. It has been speculated that autophagy is down-regulated in a number of diseases, for example, cystic fibrosis (Luciani et al. (2011) AUTOPHAGY, 7, p. 104-106).

Cystic fibrosis (CF) has been described as one of the most common, life-shortening autosomal recessive hereditary diseases in the Caucasian population. It is an orphan disease that affects approximately 30,000 children and adults in the U.S. (70,000 worldwide); and about 1,000 new cases are diagnosed each year. The disease is characterized by mutations in the cystic fibrosis transmembrane conductance regulator (CFTR), which results in either loss or impaired ability to transport chloride ions by various secretory and absorptive epithelial cells in the lung, pancreas, liver, and intestine (see, for example, Derichs (2013) EUR. RESP. REV, 22, p. 58-65). The resulting decrease in anion transport and imbalance in fluid homeostasis produce thick and viscous mucus in the lungs, which can obstruct airways, causing chronic inflammation and infection. This leads to a progressive decline in lung function and a limited life expectancy in patients with the more severe form of the disease.

The CFTR is a cAMP-activated ATP-gated ion channel composed of approximately 1,480 amino acids. The protein consists of 5 domains: two transmembrane domains, each containing 6 spans of alpha helices. Each transmembrane domain is connected to a nucleotide binding domain (NBD). The first NBD is connected to the second transmembrane domain by a regulatory “R” domain. The gene encoding CFTR was reported in year 1989 (see, Rommens et al. (1989) SCIENCE, 245, p. 1059-1065). Since then, over 1900 sequence variations in the CFTR gene have been identified, the majority of which fall into one of the following 6 classes: Class I mutations result from non-sense and frame shift mutations, which reduce the quantity of the CFTR; Class II mutations have folding defects which result in premature degradation; Class III mutations result in limited channel gating; Class IV mutations have conductance defects; Class V mutations have a transcriptional defect that results in a reduced quantity of the CFTR being produced; Class VI mutations have a high turnover of the CFTR at the channel surface (see, for example, Rowntree and Harris (2003) ANN. HUM. GENET., 67, p. 471-485; Zielenski (2000) RESPIRATION, 67, p. 117-133; and MacDonald et al. (2007) PAEDIATRIC DRUGS, 9, p. 1-10).

To manifest the debilitating CF disease, an individual inherits two defective CFTR alleles, one from each parent. Of the over 1900 sequence variations in the CFTR that have been identified, the following 4 mutations have a worldwide prevalence of around 1-3% each: G551D, W1282X, G542X and N1303K. The most prevalent CFTR mutation, with an allelic frequency of about 90% worldwide, is the ΔF508 mutation (a Class II mutation, deletion of a phenylalanine which causes protein mis-folding and premature degradation). The ΔF508 deletion mutation can be manifested in either homozygous or heterozygous form.

Research on therapeutic interventions has identified several anti-inflammatory and anti-infective therapies useful in controlling certain debilitating symptoms of CF (see, for example, Nichols et al. (2008) CLINIC REV. ALLERG. IMMUNOL., 35, p. 135-153). More recently, disease-modifying therapies have been introduced to address the defective CFTR. CFTR “potentiators” were designed to increase the open probability of CFTR channels that are available at the membrane but have gating (Class III) and conductance (Class IV) mutations. Ivacaftor (VX-770) is a CFTR potentiator that received FDA approval for the treatment of CF patients with gating mutations that included G551D, G178R, S549N, S549R, G551S, G124E, S1251N, S1255P, and G1349D (see, for example, Van Goor et al. (2009) PROC. NATL. ACAD. SCI. USA 106, p. 18825-18830). However, patients with these gating mutations represent only a small percentage of CF patients worldwide.

In addition to CFTR potentiators, clinical developments have been reported evaluating the potential of a CFTR “corrector” to increase the amount of CFTR that can be delivered to the cell membrane. VX-809 (Lumacaftor) is a CFTR corrector that has recently been approved by the FDA, when used in combination with Ivacaftor, in CF patients with homozygous ΔF508 mutation (see, for example, Van Goor et al. (2011) PNAS, 108, p. 18843-18848; and Ren et al. (2013) MOL. BIOL. CELL, 24, p. 3016-3024).

Other ways of potentially “correcting” the mis-folded ΔF508 CFTR protein is to use a molecular chaperone (Chanoux and Rubenstein (2012) FRONTIERS IN PHARMACOLOGY, 3; doi. 10.3389/fpharm. 2012.00137) or a small molecule inhibitor of the enzyme S-nitrosoglutathione reductase (GSNOR). GSNOR inhibitors have been shown to have efficacy in pre-clinical animal models of asthma, chronic obstructive pulmonary disease and inflammatory bowel disease (Green et al. (2012) BIOCHEMISTRY, 51, p. 2157-2168). GSNOR inhibitors have recently been shown to have a modest effect in rescuing the mis-folded ΔF508 CFTR protein when used in combination with VX-809 (Angers et al. (2014) “Pharmacological correction and acute inhibition of GSNOR results in improved in vitro CFTR function” Abstracts from the 28^{th} Annual N. America CF Conference, Atlanta, Ga.).

The mitochondria has often been referred to as the powerhouse for cells since it contains the respiratory assembly and the various enzymes needed for the citric acid cycle and the fatty acid oxidation. Oxidative phosphorylation takes place in the inner mitochondrial membrane to produce ATP, an energy source that is needed to power the various cellular functions. A dysregulation of mitochondrial function in any form leads to diseases; and some of these include neurodegenerative and muscular disorders, ischemia and reperfusion injury, diabetes, obesity, inherited mitochondrial diseases, and even cancer. For example, Yao et al. (PROC. NAT. ACAD. SCI. (2009) 106, p. 14670-14675) have reported that mitochondrial dysfunction plays a central role in the pathogenesis of neurodegenerative diseases such as Alzheimer's disease. This disease's pathology is accompanied by a decrease in the expression and activity of the various enzymes that are important for mitochondrial bioenergetics; and this, in turn, leads to a decrease in the synthesis of ATP. In CF patients, the defective CFTR can also lead to various mitochondrial dysfunctions, including differential gene expression, alterations in oxidative phosphorylation, calcium homeostasis, oxidative stress, apoptosis and innate immune response (for a review, see Valdivieso and Santa-Coloma, REDOX BIOLOGY (2013) 1, p. 190-202).

Despite the efforts made to date, there is still an ongoing need for additional compositions and methods for treating CF, and in particular certain forms of CF associated with mutations that are difficult to treat using existing therapies.

### SUMMARY OF THE INVENTION

The invention provides methods and compositions for treating various medical diseases associated with dysregulation of autophagy or mitochondrial bioenergetics, for example, disorders where either autophagy or mitochondrial bioenergetics is reduced relative to subjects without the disorder. The invention is based, in part, upon the discovery that fatty acid cysteine-based conjugates are useful in activating autophagy, and that the conjugate can be used treat a variety of human diseases such as CF.

The conjugates of the invention are also useful in improving mitochondrial bioenergetics and this can be useful in treating a variety of diseases. An agent that can improve the mitochondrial bioenergetics can, therefore, be of significant benefit to CF patients or to those with mitochondrial dysfunction. The transcription factor Nrf2 and its repressor Keap1 are known to regulate a large network of antioxidant genes. Holmstrom et al. have demonstrated that loss of Nrf2 can lead to mitochondrial depolarization, decreased ATP levels and impaired respiration (BIOLOGY OPEN (2013) 2, p. 761-770). Conversely, activation of Nrf2 increases the mitochondrial membrane potential and ATP levels, the rate of respiration and the efficiency of oxidative phosphorylation. An agent that can effectively activate the Nrf2 pathway can potentially be used to improve the mitochondrial bioenergetics.

The conjugates of the invention can synergistically activate the Nrf2 pathway; and this activity cannot be replicated by administering the individual components (e.g., cysteine and fatty acid) or a combination of the individual components. Furthermore, when the conjugates are coupled with the appropriate CFTR modulator, the resulting conjugates can be used to treat CF. The conjugates described herein have therapeutic effects that cannot be achieved by administering the CFTR modulator, cysteine, or a fatty acid separately or as a combination of individual components. The covalent linkage of the CFTR modulator, cysteine, and a fatty acid, for example, an omega-3 fatty acid, allows the simultaneous delivery of the three components to an intracellular location, whereupon the individual components are released by cleavage (e.g., enzymatic cleavage) at the location and at the same time. In addition, fatty acid cysteine-based conjugates can also be covalently linked to agents that target the mitochondria, to further enhance the delivery of the covalent conjugates to the desired site of action.

One benefit of the invention is that administration of the fatty acid cysteine-based conjugate results in a greater lever of activation of autophagy than can be achieved by administering the components individually. Administration of a fatty acid cysteine-based conjugate can cause a synergistic decrease in inflammation and an increase in CFTR function at a much lower concentration than the individual components, or as a combination of the individual components. Furthermore, administration of a fatty acid cysteine-based conjugate can result in an increase Nrf2 activity and in the mitochondrial bioenergetics; and this effect cannot be replicated by administering the individual components or a combination of the individual components. Thus, the fatty acid cysteine-based conjugates provide multiple benefits that cannot be achieved by separate administration of individual components (separately or co-administered) that are conjugated to produce the fatty acid cysteine conjugate.

Exemplary fatty acid cysteine-based conjugates are described herein using generic and specific chemical formulae. For example, in one aspect, the invention provides a family of fatty acid cysteine-based conjugates by Formula I:

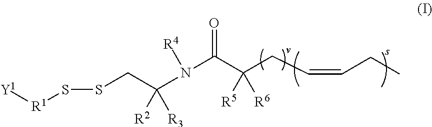

or a pharmaceutically acceptable salt or solvate thereof; wherein the variables are as defined in the detailed description.

In another aspect, the invention provides a family of fatty acid cysteine-based conjugates of Formula II:

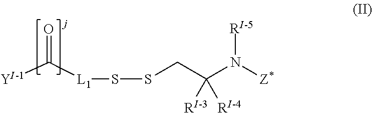

or a pharmaceutically acceptable salt or solvate thereof; wherein the variables are as defined in the detailed description.

In another aspect, the invention provides a family of cysteine conjugates embraced of Formula III:

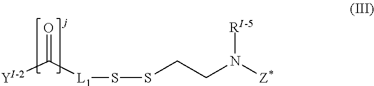

wherein the variables are as defined in the detailed description.

In another aspect, the invention provides a family of fatty acid cysteine-based conjugates of Formula IV:

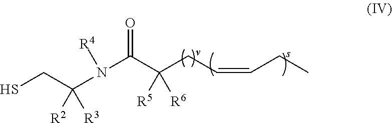

or a pharmaceutically acceptable salt or solvate thereof; wherein the variables are as defined in the detailed description.

Additional generic formulae and specific fatty acid cysteine-based conjugates are described in the detailed description and examples.

Another aspect of the invention provides a method of treating a disease described herein, such as CF, idiopathic pulmonary fibrosis (IPF), a neurodegenerative disease, inflammatory disease, liver disease, muscle disease, infection, mitochondria disease or immune disease. The method comprises administering to a subject in need thereof a therapeutically effective amount of a fatty acid cysteine conjugate, such as a compound of Formula I, Formula II, Formula III, or Formula IV, to treat the disease. Exemplary neurodegenerative diseases include Huntington's disease, Parkinson's disease, Alzheimer's disease, and transmissible spongiform encephalopathies. In certain embodiments, the disease to be treated is CF.

Another aspect of the invention provides a method of activating autophagy in a patient. The method comprises administering to a subject in need thereof an effective amount of a fatty acid cysteine conjugate described herein, such as a compound of Formula I, Formula II, Formula III, or Formula IV, to activate autophagy in the subject. In certain embodiments, the subject suffers from CF, a neurodegenerative disease, or inflammatory disease.

Another aspect of the invention provides a method of improving mitochondrial bioenergetics in a patient. The method comprises administering to a subject in need thereof an effective amount of a fatty acid cysteine conjugate described herein, such as a compound of Formula I, Formula II, Formula III, or Formula IV, to improve mitochondrial bioenergetics in the subject. In certain embodiments, the subject suffers from CF, a neurodegenerative disease, inflammatory disease or mitochondrial disease.

Pharmaceutical compositions that comprise a fatty acid cysteine conjugate (for example, the conjugate of Formula I or Formula II or Formula III or Formula IV) and a pharmaceutically acceptable carrier are provided. The compositions are useful for treating a disease by activating autophagy or improving mitochondrial bioenergetics.

Various aspects and embodiments of the invention are described in more detail below. Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise.

### BRIEF DESCRIPTION OF THE FIGURES

FIG. 1A is a graph showing the oxygen consumption rate (“OCR”) over time when C2C12 cells were treated for 4 hours with the following treatment groups prior to treatment with 500 μM of H₂O₂: 1) vehicle group (labelled “H₂O₂”); 2) 1 μM of compound I-1; 3) 10 μM of compound I-1; 4) positive control consisting of 5 mM of N-acetyl cysteine (NAC); 5) positive control consisting of 5K U/mL of the enzyme catalase; 6) a control group that was not treated with H₂O₂.

FIG. 1B is a bar chart summarizing the data shown in FIG. 1A with the OCR data corrected for the amount that was inhibited by the addition of the mitochondrial inhibitor antimycin A.

FIG. 2A is a graph showing the OCR data over time when C2C12 cells were treated for 24 hours with the following treatment groups prior to treatment with 500 μM of H₂O₂:1) vehicle group (labelled “H₂O₂”); 2) 1 μM of compound I-1; 3) 10 μM of compound I-1; 4) positive control consisting of 5 mM of N-acetyl cysteine (NAC); 5) positive control consisting of 5K U/mL of the enzyme catalase; 6) a control group that was not treated with H₂O₂.

FIG. 2B is a bar chart summarizing the data shown in FIG. 2A with the OCR data corrected for the amount that was inhibited by the addition of the mitochondrial inhibitor antimycin A.

FIG. 3A shows the OCR data over time when C2C12 cells were treated for 4 hours with the following treatment groups prior treatment with 1 mM of H₂O₂: 1) vehicle group (labelled “H₂O₂”); 2) 1 μM of compound I-3; 3) 10 μM of compound I-3; 4) positive control consisting of 5 mM of N-acetyl cysteine (NAC); 5) positive control consisting of 5K U/mL of the enzyme catalase; 6) a control group that was not treated with H₂O₂.

FIG. 3B is a bar chart summarizing the data shown in FIG. 3A with the OCR data corrected for the amount that was inhibited by the addition of the mitochondrial inhibitor antimycin A.

FIG. 4A is a graph showing the OCR data over time when C2C12 cells were treated for 4 hours with the following treatment groups prior treatment with 1 mM of H₂O₂: 1) vehicle group (labelled “H₂O₂”); 2) 1 μM of compound I-5; 3) 10 μM of compound I-5; 4) positive control consisting of 5 mM of N-acetyl cysteine (NAC); 5) positive control consisting of 5K U/mL of the enzyme catalase; 6) a control group that was not treated with H₂O₂.

FIG. 4B is a bar chart summarizing the data shown in FIG. 4A with the OCR data corrected for the amount that was inhibited by the addition of the mitochondrial inhibitor antimycin A.

FIG. 5A is a bar chart showing the ΔmRNA/HPRT level for the Gclc gene vs concentration of compound I-1.

FIG. 5B is a bar chart showing the ΔmRNA/HPRT level for the Gclm gene vs concentration of compound I-1.

FIG. 5C is a bar chart showing the ΔmRNA/HPRT level for the Gsta2 gene as a function of the concentration of compound I-1.

FIG. 5D is a bar chart showing the ΔmRNA/HPRT level for the HMOX gene as a function of the concentration of compound I-1.

FIG. 5E is a bar chart showing the ΔmRNA/HPRT level for the NQO1 gene as a function of the concentration of compound I-1.

FIG. 5F is a bar chart showing the ΔmRNA/HPRT level for the SRXN1 gene as a function of the concentration of compound I-1.

FIG. 5G is a bar chart showing the ΔmRNA/HPRT level for the TXNRD1 gene as a function of the concentration of compound I-1.

FIG. 6 is a bar chart showing the results for a synergy experiment for the fatty acid cysteine conjugate I-1 using the ΔmRNA/HPRT level for the NQO1 gene compared to NACA, DHA, and the combination of NACA and DHA.

### DETAILED DESCRIPTION

The invention provides methods and compositions for activating autophagy and treating various medical diseases, in particular diseases associated with autophagy dysregulation. The invention is based, in part, upon the discovery that fatty acid cysteine-based conjugates are useful in activating autophagy, and can be used to treat a variety of human diseases, for example, CF. Fatty acid cysteine-based conjugates described herein have therapeutic effects that cannot be achieved by administering the three individual components separately or in combination. When coupled together with a CFTR modulator, a fatty acid cysteine conjugate can be useful for treating CF.

The covalent linkage of a CFTR modulator, cysteine, and an omega-3 fatty acid allows the simultaneous delivery of the three components to an intracellular location, whereupon the individual components are released by cleavage (e.g., enzymatic cleavage) at the location and at the same time. A benefit of the invention is that administration of the fatty acid cysteine-based conjugate results in a greater lever of activation of autophagy than can be achieved by administering the components individually. Furthermore, administration of the fatty acid cysteine-based conjugate can cause a synergistic decrease in inflammation and an increase in CFTR function at a much lower concentration than the three individual components administered alone, or in combination with the fatty acid. As a result, fatty acid cysteine-based conjugates provide multiple benefits that cannot be achieved by separate administration of individual components (either separately or co-administered) that make up fatty acid cysteine conjugate. The fatty acid cysteine-based conjugates and the therapeutic methods described herein are contemplated to have particular advantages in treating CF.

CF is an orphan disease that affects some 30,000 patients in the U.S. It is a debilitating disease that is associated with a genetic mutation that leads to a defective CFTR, an ion channel that transports chloride ions across epithelial cell membranes. Patients with CF have been shown to have a defective and decreased level of autophagy, an evolutionarily conserved lysosomal degradation pathway that serves as a means to help cells remove extraneous or damaged organelles, defective or mis-folded proteins and even invading microorganisms. Activating autophagy has been shown to be potentially useful in restoring function to a defective CFTR.

The fatty acid cysteine-based conjugates described herein can improve mitochondrial bioenergetics and this can be useful in treating a variety of diseases. The fatty acid cysteine-based conjugates of the invention synergistically activate the Nrf2 pathway and this can provide cytoprotection as well as the substrate that is needed for mitochondrial respiration.

It is contemplated that the activation of autophagy is also useful for the treatment of a variety of diseases other than CF, for example, diseases associated with reduced autophagy in cells, tissues, organelles, organs. Such diseases include, for example, idiopathic pulmonary fibrosis (IPF), pulmonary hypertension (PH), neurodegenerative diseases, liver diseases, muscle diseases, cardiac diseases, metabolic diseases, infection, immunity and inflammatory diseases. Pulmonary hypertension includes pulmonary arterial hypertension (WHO group I, idiopathic, heritable and drug/toxin-induced PH), pulmonary hypertension due to systolic or diastolic dysfunction, valvular heart disease (WHO group II) and pulmonary hypertension of other classifications that include those from WHO group III-V. Liver diseases include non-alcoholic fatty liver disease (NAFLD), non-alcoholic steatohepatitis (NASH), NASH cirrhosis and hepatocellular carcinoma (HCC). An example of a metabolic disease that can be treated with a fatty acid cysteine conjugate includes type 2 diabetes, which is commonly observed among CF patients. Neurodegenerative diseases include Huntington's disease, Parkinson's disease, Alzheimer's disease, and transmissible spongiform encephalopathies. Autophagy restoration therapy could also be useful for diseases such as Vici syndrome, sarcopenia and muscular dystrophy. There are multiple forms of muscular dystrophy and these include Duchenne muscular dystrophy, which is most common. Other forms of muscular dystrophy include Becker, limb-girdle, congenital, facioscapulohumeral, myotonic, oculopharyngeal, distal and Emery-Dreifuss muscular dystrophy. In addition, other diseases that have defective autophagy include age-related macular degeneration, Danon disease, X-linked myopathy, infantile autophagic vacuolar myopathy, adult onset vacuolar myopathy, Pompe disease, sporadic inclusion body myositis, limb girdle muscular dystrophy type 2B, and Miyoshi myopathy. Fatty acid cysteine-based conjugates may also useful for the treatment of mitochondrial diseases such as Leigh Syndrome, Diabetes Mellitus and Deafness (DAD), Leber's hereditary optic neuropathy, Neuropathy-ataxia-retinis pigmentosa and ptosis (NARP), myoneurogenic gastrointestinal encephalopathy (MNGIE), myoclonic epilepsy with ragged red fibers (MERRF), and mitochondrial myopathy-encephalomyopathy-lactic acidosis-stroke like symptoms (MELAS). Since cysteamine is being released intracellularly, the compounds of the invention may also be used to treat the lysosomal disorder nephropathic cystinosis.

Unless otherwise indicated, the practice of the present invention employs conventional techniques of organic chemistry, cell biology, biochemistry, pharmacology, formulation and drug delivery. Various aspects of the invention are set forth below in sections for clarity; however, it is understood that aspects of the invention described in one particular section are not to be limited to any particular section.

### I. Definitions

To facilitate an understanding of the present invention, a number of terms and phrases are defined below.

The articles “a” and “an” are used in this disclosure to refer to one or more than one (i.e., to at least one) of the grammatical object of the article, unless the context is inappropriate. By way of example, “an element” means one element or more than one element.

The term “and/or” is used in this disclosure to mean either “and” or “or” unless indicated otherwise.

The term “alkyl” as used herein refers to a saturated straight or branched hydrocarbon, such as a straight or branched group of 1-12, 1-10, or 1-6 carbon atoms, referred to herein as C₁-C₁₂alkyl, C₁-C₁₀alkyl, and C₁-C₆alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “C₁-C₃ alkyl” refers to a straight or branched chain saturated hydrocarbon containing 1-3 carbon atoms. Examples of a C₁-C₃ alkyl group include, but are not limited to, methyl, ethyl, propyl and isopropyl. The term “C₁-C₄ alkyl” refers to a straight or branched chain saturated hydrocarbon containing 1-4 carbon atoms. Examples of a C₁-C₄ alkyl group include, but are not limited to, methyl, ethyl, propyl, butyl, isopropyl, isobutyl, sec-butyl and tert-butyl. The term “C₁-C₅ alkyl” refers to a straight or branched chain saturated hydrocarbon containing 1-5 carbon atoms. Examples of a C₁-C₅ alkyl group include, but are not limited to, methyl, ethyl, propyl, butyl, pentyl, isopropyl, isobutyl, sec-butyl and tert-butyl, isopentyl and neopentyl. The term “C₁-C₆ alkyl” refers to a straight or branched chain saturated hydrocarbon containing 1-6 carbon atoms. Examples of a C₁-C₆ alkyl group include, but are not limited to, methyl, ethyl, propyl, butyl, pentyl, hexyl, isopropyl, isobutyl, sec-butyl, tert-butyl, isopentyl, and neopentyl.

The term “hydroxyalkyl” refers to an alkyl group that is substituted with at least one hydroxyl group. In certain embodiments, the hydroxyalkyl group is an alkyl group that is substituted with one hydroxyl group. In certain other embodiments, the hydroxyalkyl group is an alkyl group that is substituted with two or three hydroxyl groups.

The term “alkylene” refers to a diradical of an alkyl group. Exemplary alkylene groups include —CH₂—, —CH₂CH₂—, —C(CH₃)₂CH₂—, and —CH₂C(H)(CH₃)CH₂—.

The term “cycloalkyl” refers to a cyclic, saturated hydrocarbon, such as one containing 3-6 carbon atoms. The cycloalkyl may contain 3-12, 3-8, 4-8, or 4-6 ring carbon atoms, referred to herein, e.g., as “C₄₋₈cycloalkyl”. Examples of a cycloalkyl group include, but are not limited to, cyclopropyl, cyclobutyl, cyclopentyl and cyclohexyl. Unless specified otherwise, it is understood that any of the substitutable hydrogens on a cycloalkyl can be substituted with halogen, C₁-C₃ alkyl, hydroxyl, alkoxy and cyano groups. In certain embodiments, the cycloalkyl is not substituted.

Unless indicated otherwise, the term “aryl” refers to carbocyclic, aromatic hydrocarbon group having 1 to 2 aromatic rings, including monocyclic or bicyclic groups such as phenyl, biphenyl or naphthyl. Where containing two aromatic rings (bicyclic, etc.), the aromatic rings of the aryl group may be joined at a single point (e.g., biphenyl), or fused (e.g., naphthyl). The aryl group may be optionally substituted by one or more substituents, e.g., 1 to 5 substituents, at any point of attachment, such substituents include, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. In certain other embodiments, the aromatic ring is not substituted, i.e., it is unsubstituted. In certain embodiments, the aryl group is a 6-10 membered ring structure. In certain embodiments, the aryl group is a 6-10 membered carbocyclic ring structure.

The term “aralkyl” refers to an alkyl group substituted with an aryl group.

The terms “heterocyclyl” and “heterocyclic group” are art-recognized and refer to saturated, partially unsaturated, or aromatic 3- to 10-membered ring structures, alternatively 3- to 7-membered rings, whose ring structures include one to four heteroatoms, such as nitrogen, oxygen, and sulfur. The number of ring atoms in the heterocyclyl group can be specified using Cₓ-Cₓ nomenclature where x is an integer specifying the number of ring atoms. For example, a C₃-C₇heterocyclyl group refers to a saturated or partially unsaturated 3- to 7-membered ring structure containing one to four heteroatoms, such as nitrogen, oxygen, and sulfur. The designation “C₃-C₇” indicates that the heterocyclic ring contains a total of from 3 to 7 ring atoms, inclusive of any heteroatoms that occupy a ring atom position. One example of a C₃heterocyclyl is aziridinyl. Heterocycles may also be mono-, bi-, or other multi-cyclic ring systems. A heterocycle may be fused to one or more aryl, partially unsaturated, or saturated rings. Heterocyclyl groups include, for example, biotinyl, chromenyl, dihydrofuryl, dihydroindolyl, dihydropyranyl, dihydrothienyl, dithiazolyl, homopiperidinyl, imidazolidinyl, isoquinolyl, isothiazolidinyl, isooxazolidinyl, morpholinyl, oxolanyl, oxazolidinyl, phenoxanthenyl, piperazinyl, piperidinyl, pyranyl, pyrazolidinyl, pyrazolinyl, pyridyl, pyrimidinyl, pyrrolidinyl, pyrrolidin-2-onyl, pyrrolinyl, tetrahydrofuryl, tetrahydroisoquinolyl, tetrahydropyranyl, tetrahydroquinolyl, thiazolidinyl, thiolanyl, thiomorpholinyl, thiopyranyl, xanthenyl, lactones, lactams such as azetidinones and pyrrolidinones, sultams, sultones, and the like. Unless specified otherwise, the heterocyclic ring is optionally substituted at one or more positions with substituents such as alkanoyl, alkoxy, alkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, oxo, phosphate, phosphonato, phosphinato, sulfate, sulfide, sulfonamido, sulfonyl and thiocarbonyl. In certain embodiments, the heterocyclyl group is not substituted, i.e., it is unsubstituted.

The term “heteroaryl” is art-recognized and refers to aromatic groups that include at least one ring heteroatom. In certain instances, a heteroaryl group contains 1, 2, 3, or 4 ring heteroatoms. Representative examples of heteroaryl groups include pyrrolyl, furanyl, thiophenyl, imidazolyl, oxazolyl, thiazolyl, triazolyl, pyrazolyl, pyridinyl, pyrazinyl, pyridazinyl and pyrimidinyl, and the like. Unless specified otherwise, the heteroaryl ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. The term “heteroaryl” also includes polycyclic ring systems having two or more rings in which two or more carbons are common to two adjoining rings (the rings are “fused rings”) wherein at least one of the rings is heteroaromatic, e.g., the other cyclic rings may be cycloalkyls, cycloalkenyls, cycloalkynyls, and/or aryls. In certain embodiments, the heteroaryl ring is substituted at one or more ring positions with halogen, alkyl, hydroxyl, or alkoxyl. In certain other embodiments, the heteroaryl ring is not substituted, i.e., it is unsubstituted. In certain embodiments, the heteroaryl group is a 5- to 10-membered ring structure, alternatively a 5- to 6-membered ring structure, whose ring structure includes 1, 2, 3, or 4 heteroatoms, such as nitrogen, oxygen, and sulfur.

The term “alkenyl” as used herein refers to an unsaturated straight or branched hydrocarbon having at least one carbon-carbon double bond, such as a straight or branched group of 2-12, 2-10, or 2-6 carbon atoms, referred to herein as C₂-C₁₂alkenyl, C₂-C₁₀alkenyl, and C₂-C₆alkenyl, respectively. Exemplary alkenyl groups include vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl, and the like.

The term “alkynyl” as used herein refers to an unsaturated straight or branched hydrocarbon having at least one carbon-carbon triple bond, such as a straight or branched group of 2-12, 2-10, or 2-6 carbon atoms, referred to herein as C₂-C₁₂alkynyl, C₂-C₁₀alkynyl, and C₂-C₆alkynyl, respectively. Exemplary alkynyl groups include ethynyl, prop-1-yn-1-yl, and but-1-yn-1-yl.

The terms “amine” and “amino” are art-recognized and refer to both unsubstituted and substituted amines, e.g., a moiety represented by the general formula —N(R⁵⁰)(R⁵¹), wherein R⁵⁰and R⁵¹ each independently represent hydrogen, alkyl, cycloalkyl, heterocyclyl, alkenyl, aryl, aralkyl, or —(CH₂)ₘ—R⁶¹; or R⁵⁰ and R⁵¹, taken together with the N atom to which they are attached complete a heterocycle having from 4 to 8 atoms in the ring structure; R⁶¹ represents an aryl, a cycloalkyl, a cycloalkenyl, a heterocycle or a polycycle; and m is zero or an integer in the range of 1 to 8. In certain embodiments, R⁵⁰ and R⁵¹ each independently represent hydrogen, alkyl, alkenyl, or —(CH₂)ₘ—R⁶¹.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O— alkenyl, —O-alkynyl, —O—(CH₂)ₘ—R₆₁, where m and R₆₁ are described above.

The term “carbamate” as used herein refers to a radical of the form —R_{g}OC(O)N(Rₕ)—, —R_{g}OC(O)N(Rₕ)Rᵢ—, or —OC(O)NRₕRᵢ, wherein R_{g}, Rₕ and Rᵢ are each independently alkoxy, aryloxy, alkyl, alkenyl, alkynyl, amide, amino, aryl, arylalkyl, carboxy, cyano, cycloalkyl, ester, ether, formyl, halogen, haloalkyl, heteroaryl, heterocyclyl, hydroxyl, ketone, nitro, sulfide, sulfonyl, or sulfonamide. Exemplary carbamates include arylcarbamates and heteroaryl carbamates, e.g., wherein at least one of R_{g}, Rₕ and Rᵢ are independently aryl or heteroaryl, such as phenyl and pyridinyl.

The symbol “ [symbol] ” indicates a point of attachment.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present invention encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise.

Individual stereoisomers of compounds of the present invention can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, or (3) direct separation of the mixture of optical enantiomers on chiral chromatographic columns. Stereoisomeric mixtures can also be resolved into their component stereoisomers by well-known methods, such as chiral-phase gas chromatography, chiral-phase high performance liquid chromatography, crystallizing the compound as a chiral salt complex, or crystallizing the compound in a chiral solvent. Further, stereoisomers can be obtained from stereomerically-pure intermediates, reagents, and catalysts by well-known asymmetric synthetic methods.

Geometric isomers can also exist in the compounds of the present invention. The symbol denotes a bond that may be a single, double or triple bond as described herein. The present invention encompasses the various geometric isomers and mixtures thereof resulting from the arrangement of substituents around a carbon-carbon double bond or arrangement of substituents around a carbocyclic ring. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers.

Substituents around a carbon-carbon double bond alternatively can be referred to as “cis” or “trans,” where “cis” represents substituents on the same side of the double bond and “trans” represents substituents on opposite sides of the double bond. The arrangement of substituents around a carbocyclic ring are designated as “cis” or “trans.” The term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

The invention also embraces isotopically labeled compounds of the invention which are identical to those recited herein, except that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the invention can generally be prepared by following procedures analogous to those disclosed in, e.g., the Examples herein by substituting an isotopically labeled reagent for a non-isotopically labeled reagent.

Unless the context suggests otherwise, the term “fatty acid cysteine conjugate” includes any and all possible isomers, stereoisomers, enantiomers, diastereomers, tautomers, pharmaceutically acceptable salts, hydrates, and solvates of the fatty acid cysteine-based conjugates described herein.

The term “any side chain of a naturally occurring amino acid” refers to a side chain of any one of the following amino acids: Isoleucine, Alanine, Leucine, Asparagine, Lysine, Aspartate, Methionine, Cysteine, Phenylalanine, Glutamate, Threonine, Glutamine, Tryptophan, Glycine, Valine, Proline, Arginine, Serine, Histidine, and Tyrosine.

The term “fatty acid” as used herein means an omega-3 fatty acid and fatty acids that are metabolized in vivo to omega-3 fatty acids. Non-limiting examples of fatty acids are all-cis-7,10,13-hexadecatrienoic acid, α-linolenic acid (ALA or all-cis-9,12,15-octadecatrienoic acid), stearidonic acid (STD or all-cis-6,9,12,15-octadecatetraenoic acid), eicosatrienoic acid (ETE or all-cis-11,14,17-eicosatrienoic acid), eicosatetraenoic acid (ETA or all-cis-8,11,14,17-eicosatetraenoic acid), eicosapentaenoic acid (EPA or all-cis-5,8,11,14,17-eicosapentaenoic acid), docosapentaenoic acid (DPA, clupanodonic acid or all-cis-7,10,13,16,19-docosapentaenoic acid), docosahexaenoic acid (DHA or all-cis-4,7,10,13,16,19-docosahexaenoic acid), tetracosapentaenoic acid (all-cis-9,12,15,18,21-docosahexaenoic acid), or tetracosahexaenoic acid (nisinic acid or all-cis-6,9,12,15,18,21-tetracosenoic acid).

The term, “cysteine” refers to a molecule having the following formula

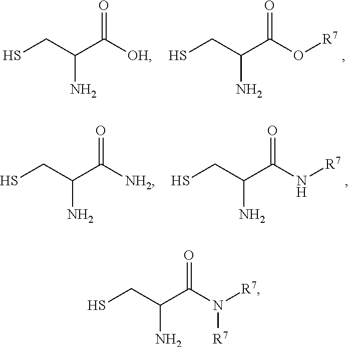

wherein R⁷ is a C₁-C₄ alkyl. In addition, the term “cysteine” can also refer to a molecule wherein the carboxyl group has been reduced to give cysteamine (also known as 2-aminoethane-1-thiol).

The term “cystic fibrosis” or “CF” refers to disorders, diseases and syndromes involving a defective CFTR. There are >1900 mutations that could lead to CF. These mutations are further divided into 6 different classes (Class I-VI). CF can refer to any of the possible mutations that could be present in any of the 6 different classes.

A “CFTR modulator” refers to a small molecule that can increase the function of a defective CFTR. A small molecule can increase the function of a CFTR by being either a CFTR potentiator or a CFTR corrector. A CFTR potentiator is useful for CFTR channels that have either gating or conductance mutations (class III or class IV CF mutations). Non-limiting examples of a CFTR potentiator include:

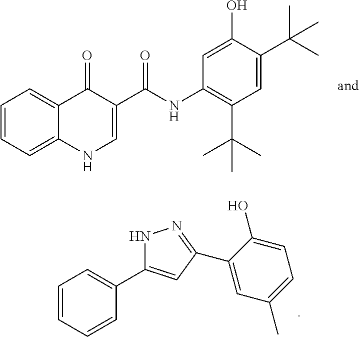

A CFTR corrector is a small molecule that can increase the amount of functioning CFTR protein to the cell membrane. Although the precise mechanism is not known in most cases, a CFTR corrector can accomplish in a number of ways, which may include 1) correcting the mis-folding protein, 2) allowing the trafficking of the defective CFTR protein to the membrane by interacting with various chaperone proteins, 3) stabilizing the defective CFTR protein at the cell membrane, 4) inhibiting S-nitrosoglutathione reductase, and/or 5) activating autophagy. Non-limiting examples of a CFTR corrector include:

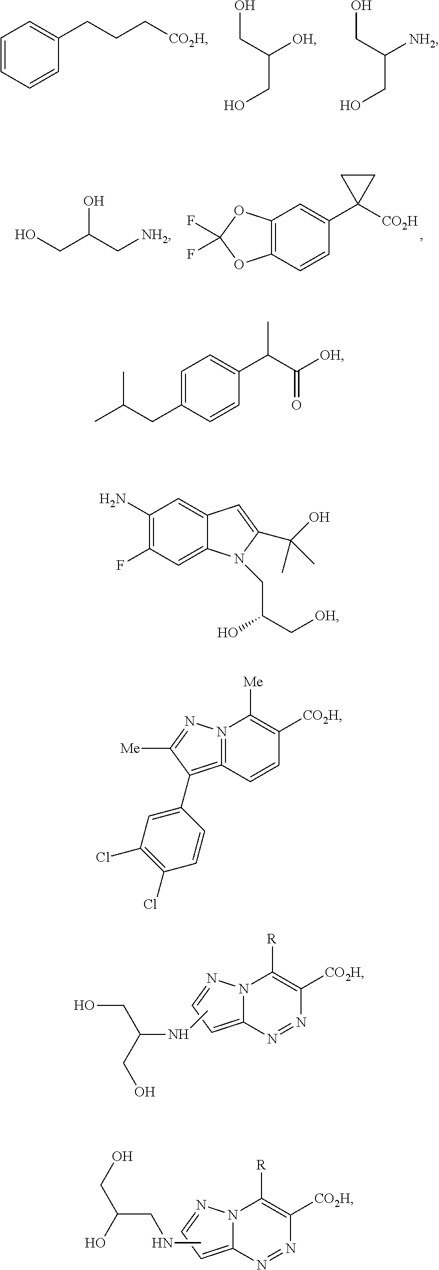

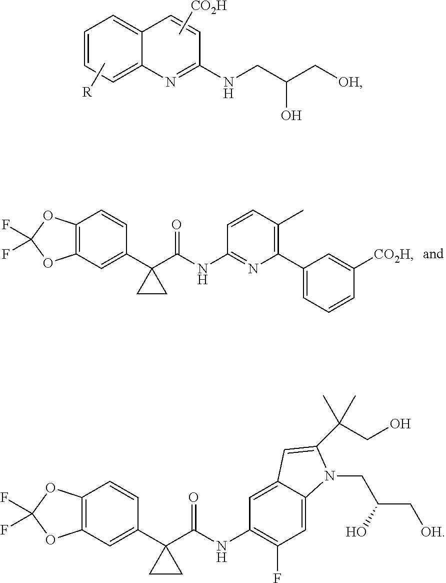

Additional non-limiting examples of a CFTR corrector can be found in WO 2007/021982, WO 2009/064959, WO 2010/053471, WO 2012/154880A1, WO 2013/112706A1, and WO 2014/210159A1.

The mitochondrial respiratory chain participates in the transfer of electrons to reduce oxygen down to water. This electron transport generates a proton gradient that is needed to drive the production of ATP by ATP synthase. This process essentially generates a negative potential across the inner mitochondrial membrane. Lipophilic cationic species can preferentially accumulate in the mitochondrial matrix at concentrations that are significantly higher than in the cytosol. Non-limiting examples of groups that can preferentially target the inner mitochondrial membrane include L-carnitive and creatine.

As used herein, the terms “patient” and “subject” refer to an organism to be treated by the methods and compositions of the present invention. Such organisms are preferably mammals (e.g., human, mouse, rat, guinea pig, dog, cat, horse, cow, pig, or non-human primate, such as a monkey, chimpanzee, baboon, rhesus, and the like), and more preferably humans.

As used herein, the term “effective amount” refers to the amount of a compound (e.g., a compound of the present invention) sufficient to effect beneficial or desired results. An effective amount can be administered in one or more administrations, applications or dosages and is not intended to be limited to a particular formulation or administration route.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see Martin, Remington's Pharmaceutical Sciences, 15th Ed., Mack Publ. Co., Easton, Pa. (1975).

As used herein, the term “pharmaceutically acceptable salt” refers to any pharmaceutically acceptable salt (e.g., acid or base) of a compound of the present invention which, upon administration to a patient, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like. For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

The term “carrier” refers to excipients and diluents, and means a material, composition or vehicle, such as a liquid or solid filler, diluent, excipient, solvent or encapsulating material, involved in carrying or transporting a pharmaceutical agent from one organ, or portion of the body, to another organ, or portion of the body.

As used herein, the term “treating” includes any effect, e.g., lessening, reducing, modulating, ameliorating or eliminating, that results in the improvement of the condition, disease, disorder, and the like, or ameliorating a symptom thereof. Treating can be curing, improving, or at least partially ameliorating the disorder. In certain embodiments, treating is curing the disease.

The term “disorder” refers to and is used interchangeably with, the terms disease, condition, or illness, unless otherwise indicated.

The following abbreviations are used herein and have the indicated definitions: Boc and BOC are tert-butoxycarbonyl, Boc₂O is di-tert-butyl dicarbonate, BSA is bovine serum albumin, CDI is 1,1′-carbonyldiimidazole, DCC is N,N′-dicyclohexylcarbodiimide, DIEA is N,N-diisopropylethylamine, DMAP is 4-dimethylaminopyridine, DMEM is Dulbecco's Modified Eagle Medium, DMF is N,N-dimethylformamide, DOSS is sodium dioctyl sulfosuccinate, EDC and EDCI are 1-ethyl-3-(3-dimethylaminopropyl)carbodiimide hydrochloride, ELISA is enzyme-linked immunosorbent assay, EtOAc is ethyl acetate, FBS is fetal bovine serum, h is hour, HATU is 2-(7-aza-1H-benzotriazole-1-yl)-1,1,3,3-tetramethyluronium hexafluorophosphate, HIV is human immunodeficiency virus, HPMC is hydroxypropyl methylcellulose, oxone is potassium peroxymonosulfate, Pd/C is palladium on carbon, TFA is trifluoroacetic acid, TGPS is tocopherol propylene glycol succinate, THF is tetrahydrofuran; HBTU is N,N,N′,N′-tetramethyl-O-(1H-benzotriazol-1-yl)uronium hexafluorophosphate; and VX-809 is the compound having the chemical name 3-{6-{[1-(2,2-difluoro-1,3-benzodioxol-5-yl)cyclopropanecarbonyl] amino}-3-methylpyridin-2-yl}benzoic acid.

Unless defined otherwise, all technical and scientific terms used herein have the same meaning as commonly understood by one of ordinary skill in the art to which this invention belongs.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

### II. Fatty Acid Cysteine-Based Conjugates

Exemplary fatty acid cysteine-based conjugates for use in the therapeutic applications and pharmaceutical compositions are described below.

### Formula I

One aspect of the invention provides a compound of Formula I represented by

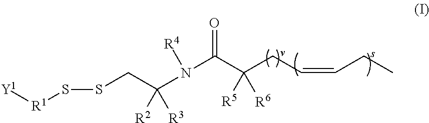

or a pharmaceutically acceptable salt or solvate thereof; wherein:

R¹ is C₁-C₆ alkylene optionally substituted by —C(O)N(R²)(hydroxyalkyl), —N(R²)C(O)-(hydroxyalkyl), —O-(hydroxyalkyl), —N(R²)-(hydroxyalkyl), —OC(O)N(R²)(hydroxyalkyl), —C(O)N(R²)(R⁸), —N(R²)C(O)R⁹, or —CO₂R⁹;

R³ is

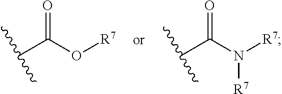

R², R⁴, and R⁷ each represent independently for each occurrence hydrogen or C₁-C₄ alkyl;

R⁵ and R⁶ are independently hydrogen, C₁-C₄ alkyl, or halogen;

R⁸ is C₁-C₆ alkyl, phenyl, benzyl, C₁-C₆ alkylene-CO₂R⁷, or C₁-C₆ alkylene-C(O)N(R⁷)₂;

R⁹ represents independently for each occurrence hydrogen, C₁-C₆ alkyl, phenyl, benzyl, C₁-C₆ alkylene-CO₂R⁷, or C₁-C₆ alkylene-C(O)N(R⁷)₂;

s is 3, 5, or 6;

v is 1, 2, or 6;

Y¹ is an amide or carbamate selected from the group consisting of —N(R⁷)C(O)-aralkyl, —N(R⁷)C(O)-(hydroxyalkyl), —C(O)N(R⁷)(hydroxyalkyl), —N(R⁷)CO₂-(hydroxyalkyl), —OC(O)N(R⁷)(hydroxyalkyl), —N(R⁷)C(O)—Z¹, and

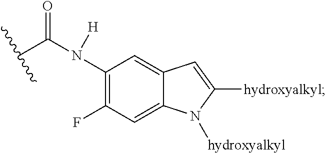

and

Z¹ is selected from the group consisting of

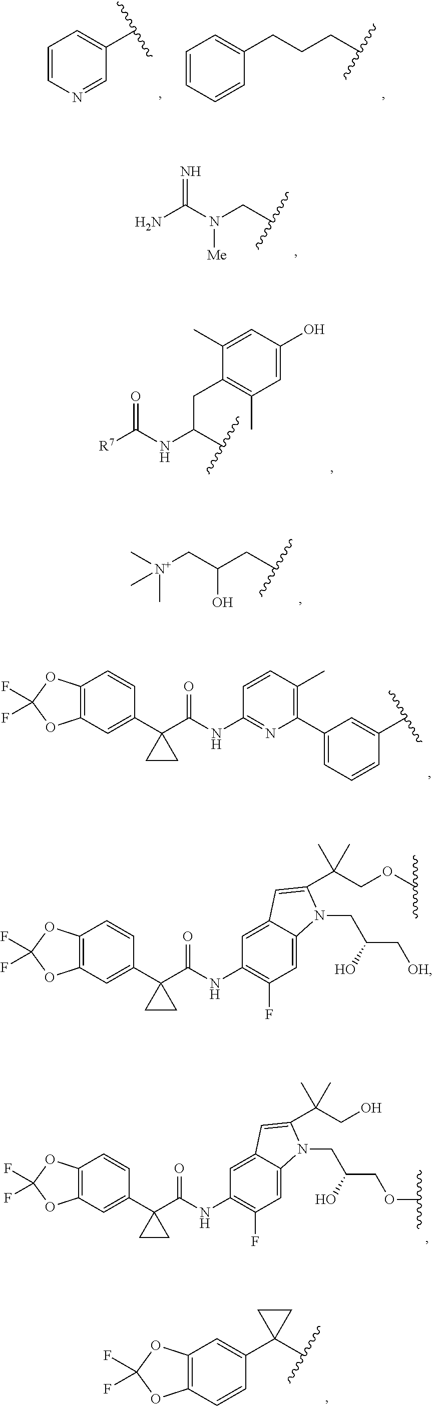

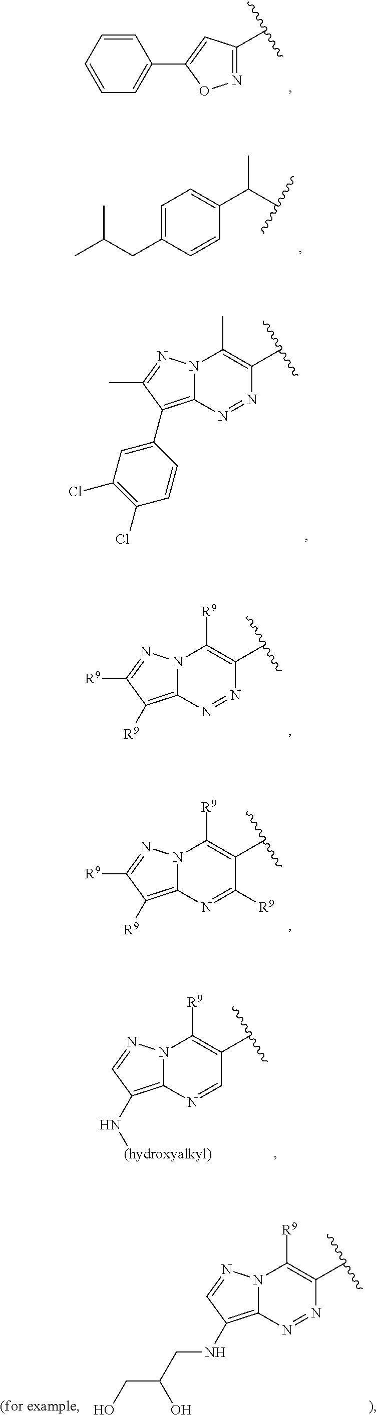

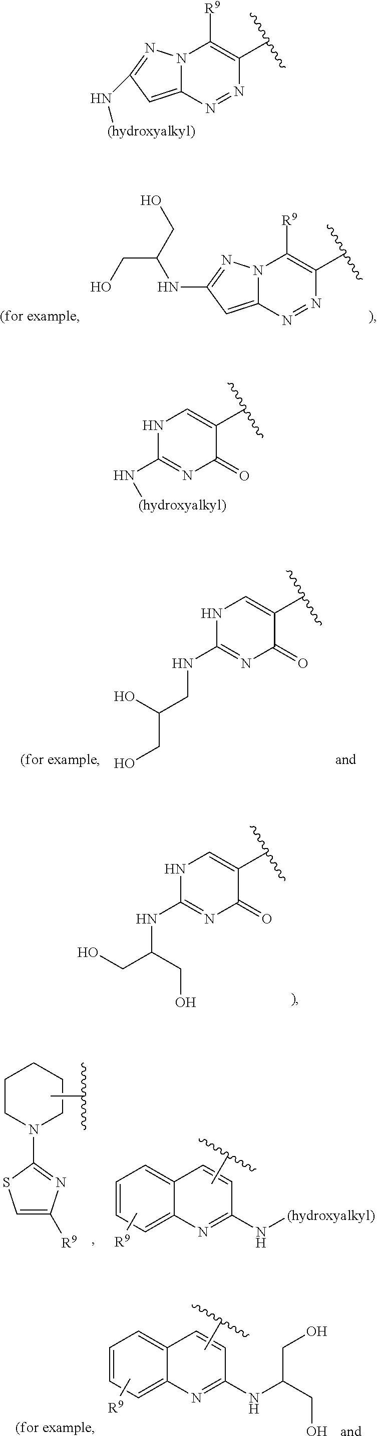

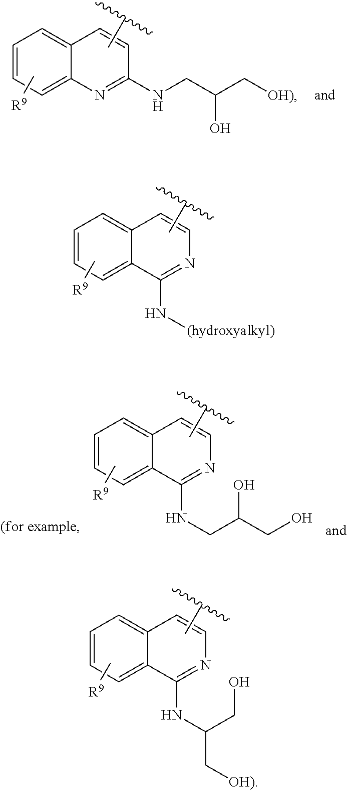

Definitions of the variables in Formula I above encompass multiple chemical groups. The application contemplates embodiments where, for example, i) the definition of a variable is a single chemical group selected from those chemical groups set forth above, ii) the definition is a collection of two or more of the chemical groups selected from those set forth above, and iii) the compound is defined by a combination of variables in which the variables are defined by (i) or (ii).

In certain embodiments, R¹ is C₁-C₆ alkylene optionally substituted by —C(O)N(R²)(hydroxyalkyl), —N(R²)C(O)(hydroxyalkyl), —O-(hydroxyalkyl), —N(R²)-(hydroxyalkyl) or —OC(O)N(R²)(hydroxyalkyl). In certain embodiments, R¹ is C₁-C₆ alkylene. In certain embodiments, R¹ is ethylene. In certain embodiments, R¹ is C₂-C₄ alkylene optionally substituted by one of the following:

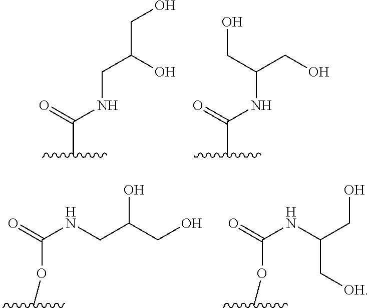 [SMILES: *C(=O)NCC(O)CO ; *C(=O)NC(CO)CO ; *OC(=O)NCC(O)CO ; *OC(=O)NC(CO)CO]

In certain embodiments, the substituent on the C₁-C₆ alkylene is attached to the same carbon atom of the C₁-C₆ alkylene as the Y¹ group.

In certain embodiments, R², and R⁴ are hydrogen.

In certain embodiments, R³ is

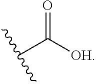 [SMILES: *C(=O)O]

In certain embodiments, R³ is

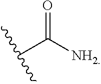 [SMILES: *C(N)=O]

In certain embodiments, R⁵ and R⁶ are hydrogen.

In certain embodiments, R⁷ is hydrogen.

In certain embodiments, s is 5. In certain embodiments, s is 6. In certain embodiments, v is 1. In certain embodiments, v is 2. In certain embodiments, v is 1, and s is 6. In certain embodiments, v is 2, and s is 5.

In certain embodiments, Y¹ is —N(R⁷)C(O)-aralkyl. In certain embodiments, Y¹ is —N(R⁷)C(O)—(CH₂)₃-phenyl. In certain embodiments, Y¹ is —N(R⁷)C(O)-(hydroxyalkyl), —C(O)N(R⁷)(hydroxyalkyl), —N(R⁷)CO₂-(hydroxyalkyl), or —OC(O)N(R⁷)(hydroxyalkyl). In certain embodiments, Y¹ is one of the following:

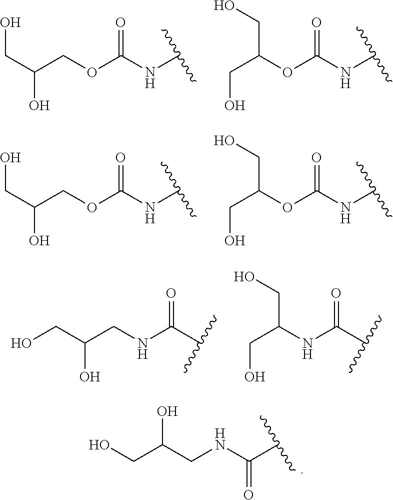

In certain embodiments, Y¹ is —N(R⁷)C(O)—Z¹.

In certain embodiments, Z¹ is

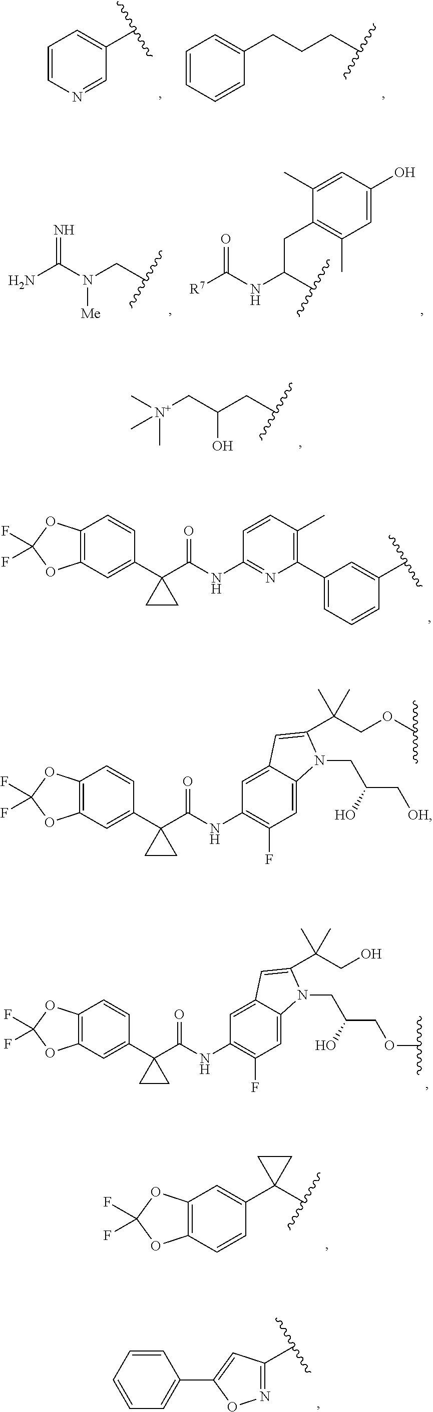

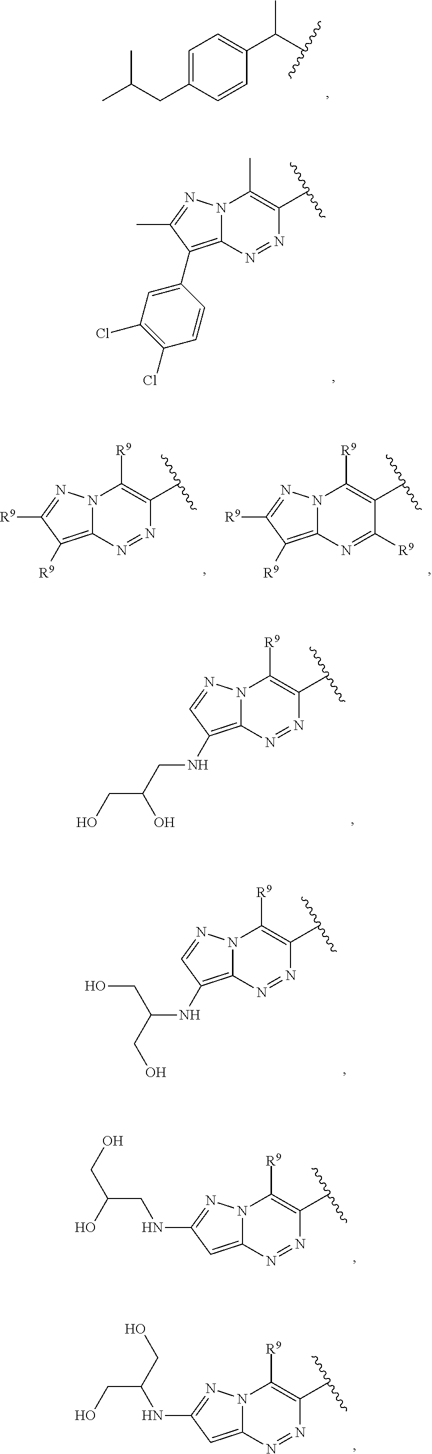

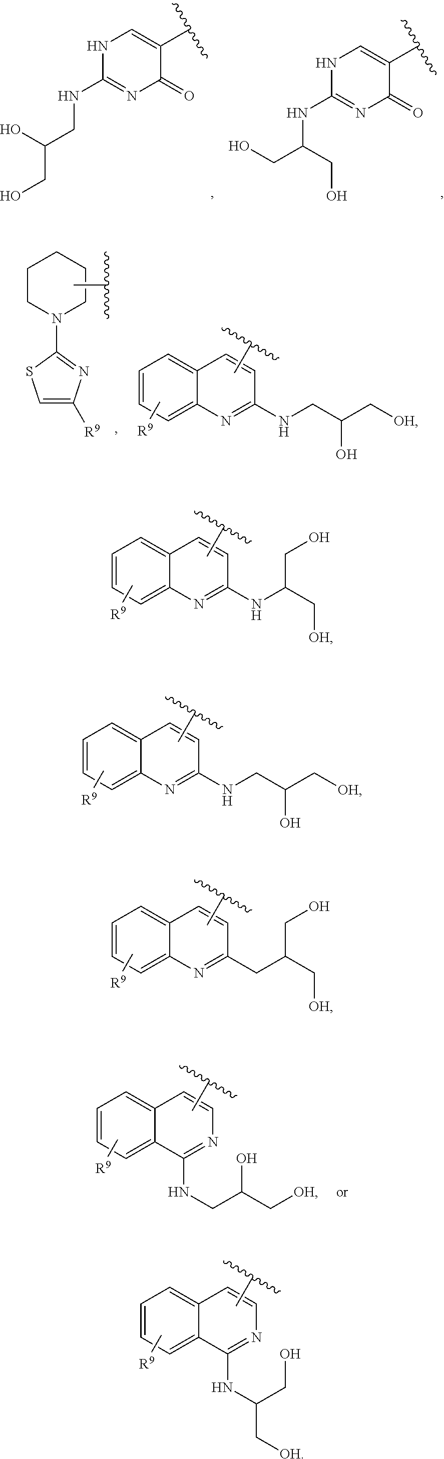

In certain embodiments, Y¹ is

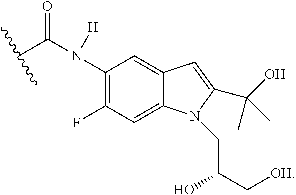 [SMILES: *C(=O)Nc1cc2cc(C(C)(C)O)n(C[C@@H](O)CO)c2cc1F]

The description above describes multiple embodiments relating to compounds of Formula I. The invention specifically contemplates all combinations of the embodiments.

### Formula II

Another aspect of the invention provides a compound of Formula II represented by:

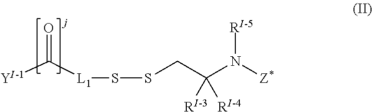

or a pharmaceutically acceptable salt or solvate thereof; wherein:

L₁ is independently

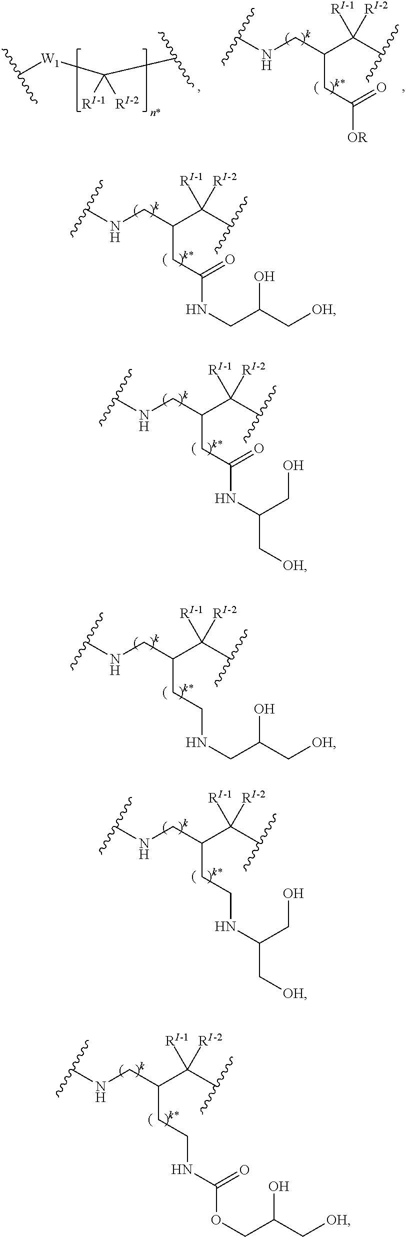

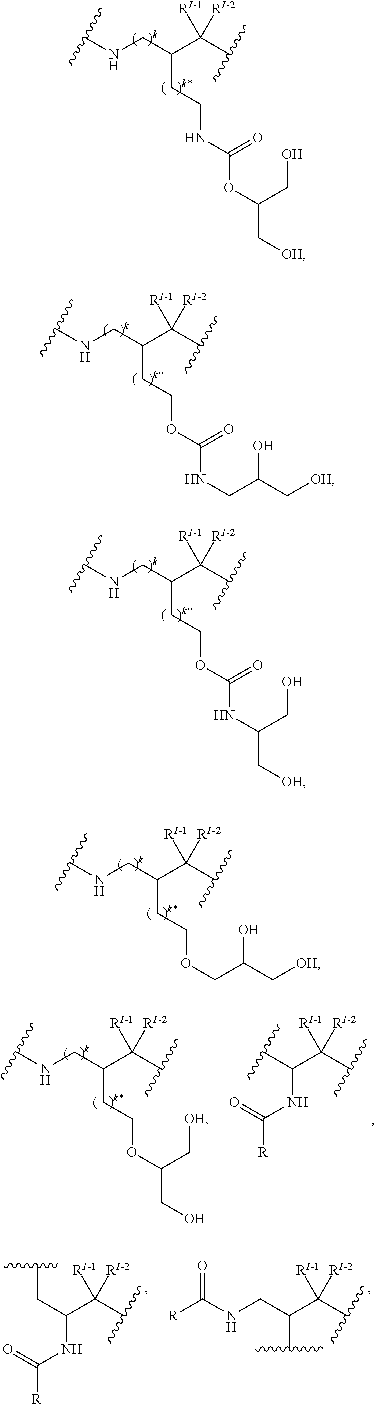

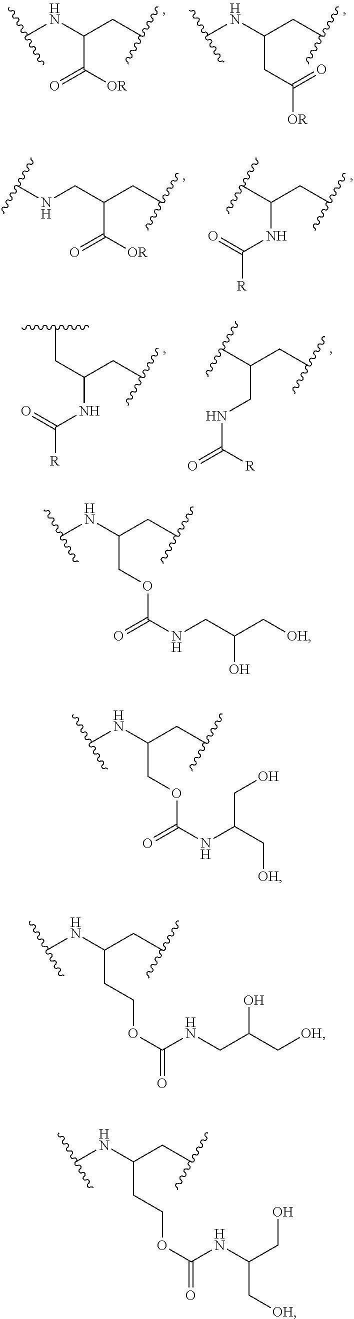

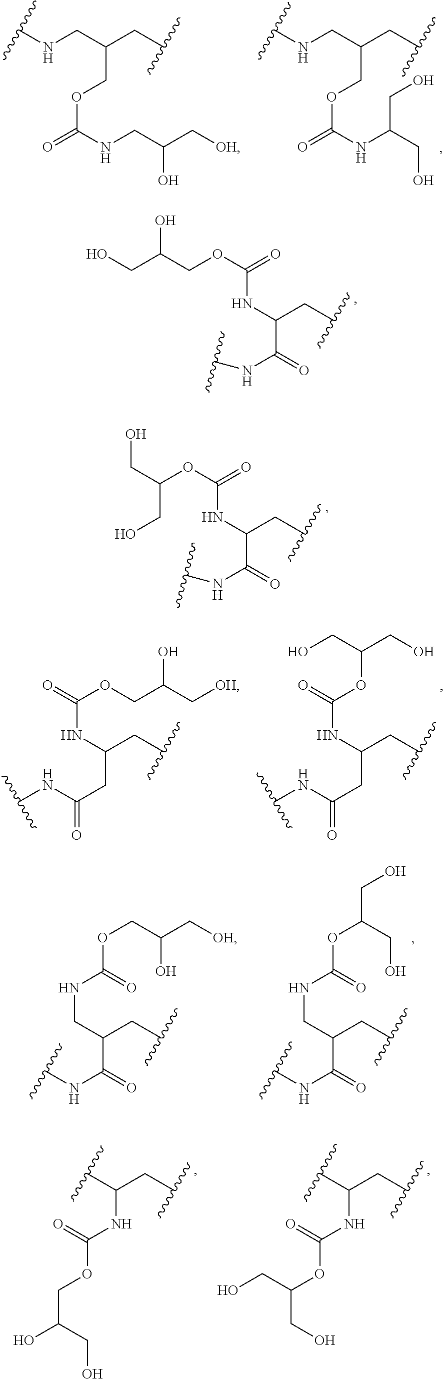

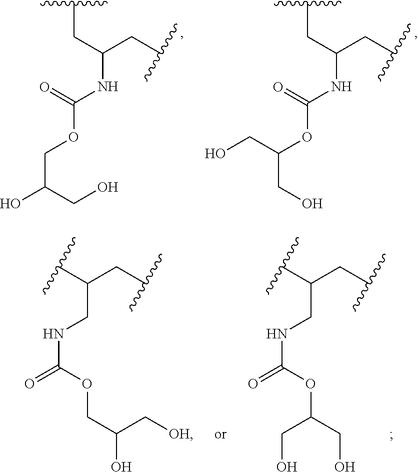 [SMILES: *CC(C*)NC(=O)OCC(O)CO ; *CC(C*)NC(=O)OC(CO)CO ; *CC(*)CNC(=O)OCC(O)CO ; *CC(*)CNC(=O)OC(CO)CO]

wherein the representation of L₁ is limited directionally left to right as is depicted, with the right hand side connected to one of the two S groups shown in Formula II;

R^{I-1}, R^{I-2}, R^{I-3}, and R^{I-5} each represent independently for each occurrence hydrogen or C₁-C₄ alkyl;

R^{I-4} is

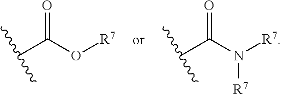

W₁ is independently a bond, O, or N(R^{I-1});

j is 0 or 1;

k and k* is independently 0 or 1;

n* is independently 1, 2, or 3, with the proviso that when n*=1 then W₁ cannot be O or NR^{I-1};

each R is independently —H, —C₁-C₆ alkyl, phenyl, benzyl, —CH₂CO₂R^{I-1}, —CH₂CON(R^{I-1})(R^{I-1});

Y^{I-1} is independently selected from

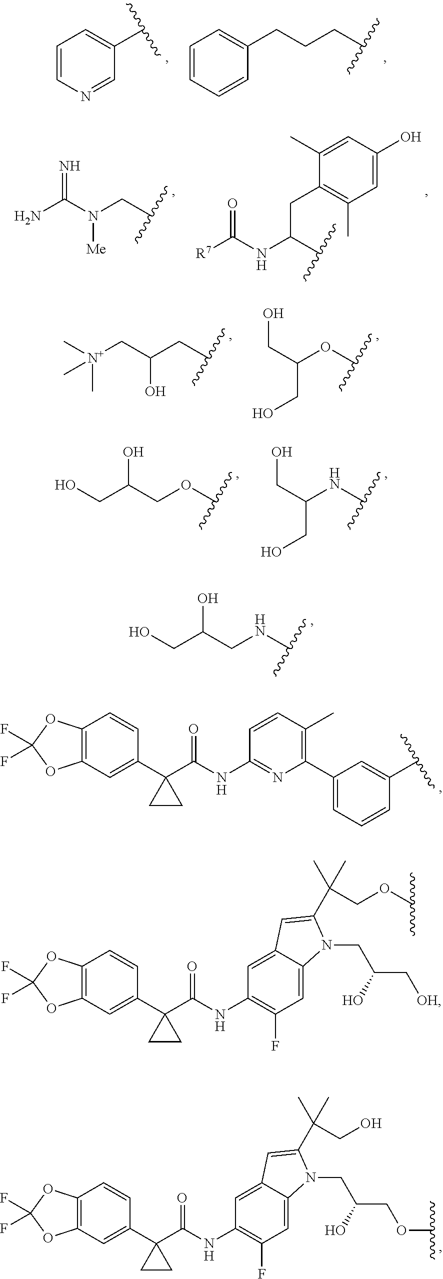

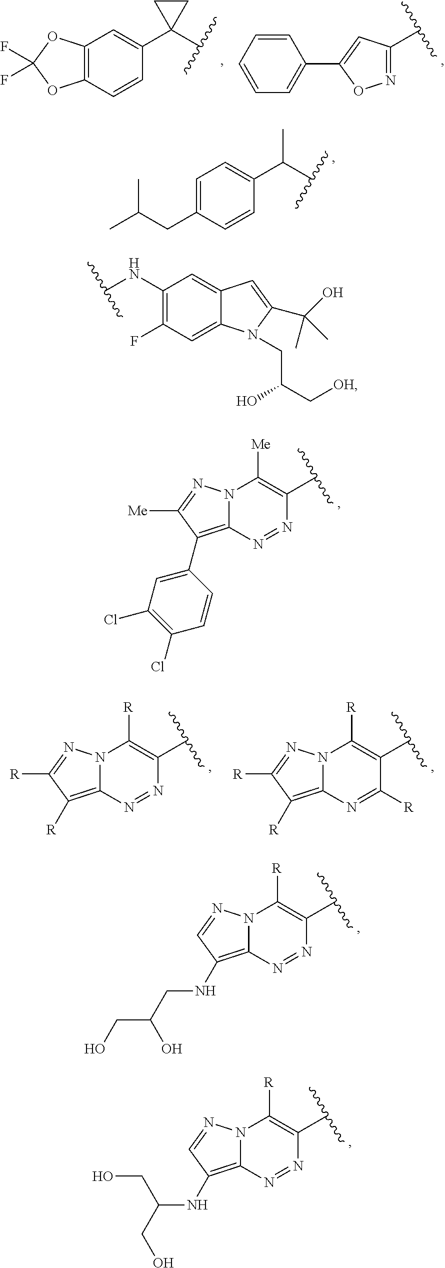

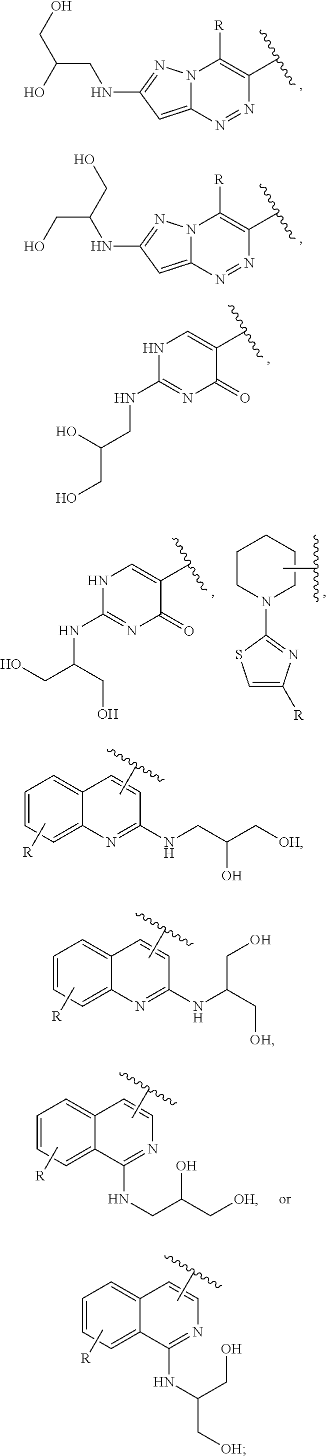

Z* is

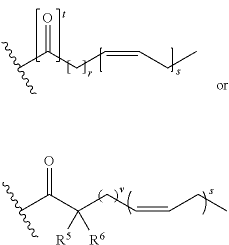

wherein:

R⁵, R⁶, and R⁷, are independently hydrogen, C₁-C₄ alkyl, or halogen;

r is 2, 3, or 7;

s is 3, 5, or 6;

t is 0 or 1; and

v is 1, 2, or 6.

### Formula III

Another aspect of the invention provides a compound of Formula III represented by:

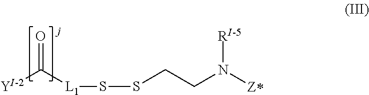

or a pharmaceutically acceptable salt or solvate thereof; wherein:

L₁ is independently

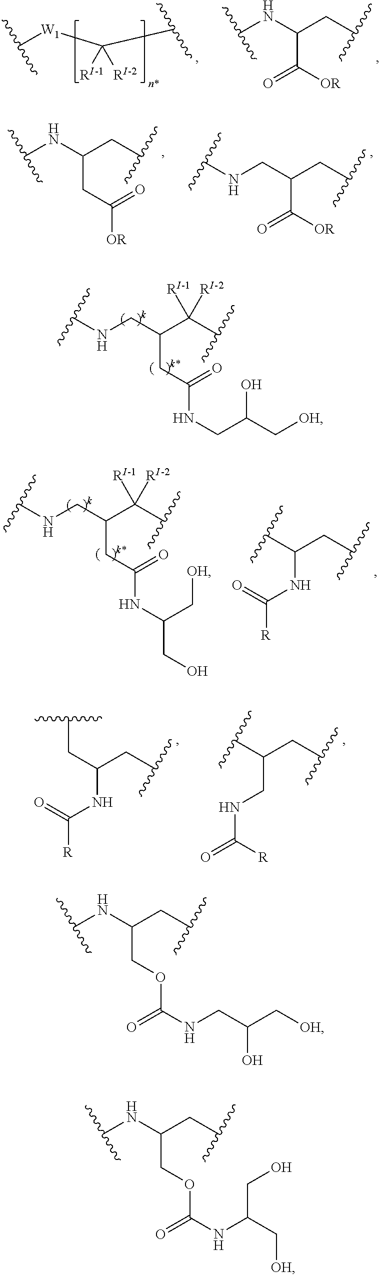

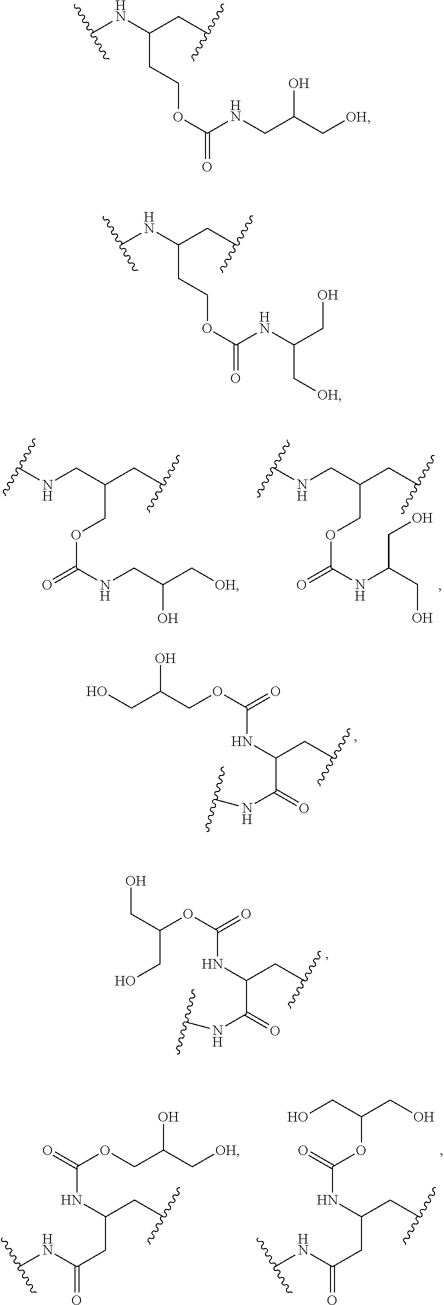

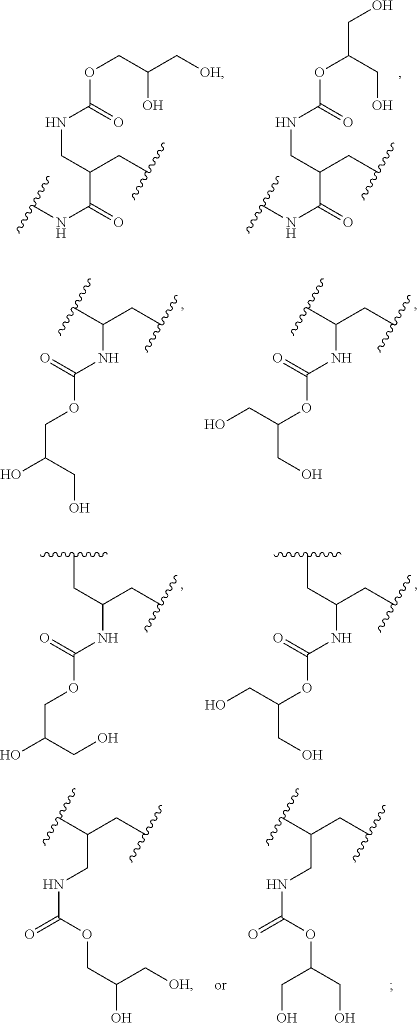

wherein the representation of L₁ is limited directionally left to right as is depicted, with the right hand side connected to one of the two S groups shown in Formula III;

R^{I-1}, R^{I-2}, and R^{I-5} each represent independently for each occurrence hydrogen or C₁-C₄ alkyl;

W₁ is independently a bond, O, or N(R^{I-1});

j is 0 or 1;

n* is independently 1, 2, or 3, with the proviso that when n*=1 then W₁ cannot be O or NR^{I-1};

each R is independently —H, —C₁-C₆ alkyl, phenyl, benzyl, —CH₂CO₂R^{I-1}, —CH₂CON(R^{I-1})(R^{I-1});

Y^{I-2} is independently selected from

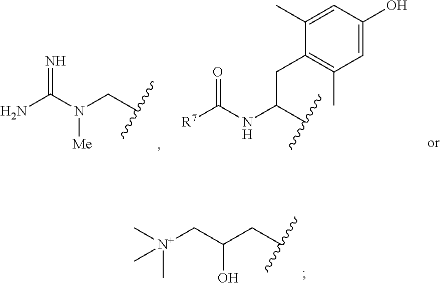

Z* is

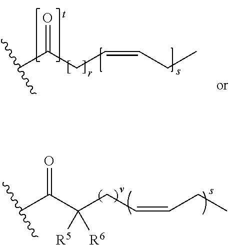

wherein:
R⁵, R⁶, and R⁷, are independently hydrogen, C₁-C₄ alkyl, or halogen;
r is 2, 3, or 7;
s is 3, 5, or 6;
t is 0 or 1; and
v is 1, 2, or 6.

Definitions of the variables in Formula III above encompass multiple chemical groups. The application contemplates embodiments where, for example, i) the definition of a variable is a single chemical group selected from those chemical groups set forth above, ii) the definition is a collection of two or more of the chemical groups selected from those set forth above, and iii) the compound is defined by a combination of variables in which the variables are defined by (i) or (ii).

In certain embodiments, the compound is a compound of (i) Formula III or a pharmaceutically acceptable salt thereof.

For the compounds of each of Formula III, the compounds can have one or more of the embodiments set forth below.

For example, in certain embodiments, R^{I-1}, R^{I-2}, and R^{I-5} each represent independently for each occurrence hydrogen or methyl. In certain embodiments, R^{I-1}, R^{I-2}, and R^{I-5} are hydrogen.

In certain embodiments, n* is 2. In certain embodiments, m* is 2. In certain embodiments, n* is 2, and m* is 2.

In certain embodiments, Y^{I-2} is

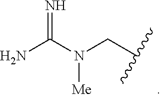 [SMILES: *CN(C)C(=N)N]

In certain embodiments, Y^{I-2} is

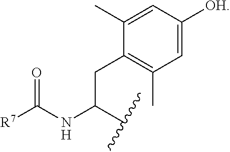

In certain embodiments, Y^{I-2} is

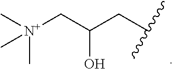 [SMILES: *CC(O)C[N+](C)(C)C]

In certain embodiments, Z* is

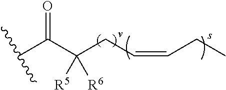

wherein R⁵ and R⁶ are hydrogen or methyl. In certain embodiments, R⁵ and R⁶ are hydrogen. In certain embodiments, Z* is one of the following:

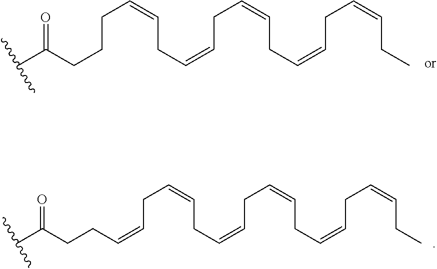 [SMILES: *C(=O)CCC/C=C\C/C=C\C/C=C\C/C=C\C/C=C\CC ; *C(=O)CC/C=C\C/C=C\C/C=C\C/C=C\C/C=C\C/C=C\CC]

The description above describes multiple embodiments relating to compounds of Formula III. The invention specifically contemplates all combinations of the foregoing embodiments.

### Formula IV

One aspect of the invention provides a compound of Formula IV represented by

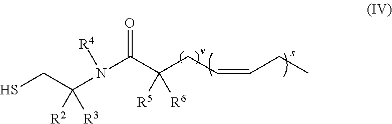

or a pharmaceutically acceptable salt or solvate thereof; wherein:

R² and R⁴ each represent independently for each occurrence hydrogen or C₁-C₄ alkyl;

R³ is

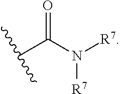

R⁵, R⁶ and R⁷ are independently hydrogen, C₁-C₄ alkyl, or halogen;

s is 3, 5, or 6; and

v is 1, 2, or 6.

In certain embodiments, the compound is a compound of (i) Formula IV or a pharmaceutically acceptable salt thereof.

In certain embodiments, R³ is

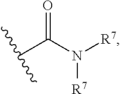

and R⁷ is independently for each occurrence hydrogen or C₁-C₄ alkyl.

In certain embodiments, R⁵ and R⁶ are H.

In certain embodiments, v is 2 and s is 6.

In certain embodiments, v is 3 and s is 5.

### Additional Fatty Acid Cysteine-Based Conjugates.

Another aspect of the invention provides a molecular conjugate comprising cysteine covalently linked via a linker to a fatty acid, wherein the fatty acid is selected from the group consisting of omega-3 fatty acids and fatty acids that are metabolized in vivo to omega-3 fatty acids. The conjugate is capable of intracellular hydrolysis to produce free cysteine and free fatty acid.

In certain embodiments, the fatty acid is selected from the group consisting of all-cis-7,10,13-hexadecatrienoic acid, α-linolenic acid, stearidonic acid, eicosatrienoic acid, eicosatetraenoic acid, eicosapentaenoic acid (EPA), docosapentaenoic acid, docosahexaenoic acid (DHA), tetracosapentaenoic acid and tetracosahexaenoic acid. In other embodiments, the fatty acid is selected from eicosapentaenoic acid and docosahexaenoic acid. In other embodiments, the fatty acid is selected from eicosapentaenoic acid and docosahexaenoic acid. In some embodiments, the fatty acid is eicosapentaenoic acid (EPA). In other embodiments, the fatty acid is docosahexaenoic acid (DHA). In some embodiments, the hydrolysis is enzymatic.

### Exemplary Compounds

In certain embodiments, the compound is one of the following compounds or a pharmaceutically acceptable salt thereof:

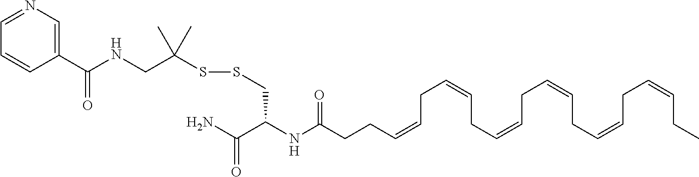 [SMILES: CC/C=C\C/C=C\C/C=C\C/C=C\C/C=C\C/C=C\CCC(=O)N[C@@H](CSSC(C)(C)CNC(=O)c1cccnc1)C(N)=O]

N-(2-(((R)-3-amino-2-((4Z,7Z,10Z,13Z,16Z,19Z)-docosa-4,7,10,13,16,19-hexaenamido)-3-oxopropyl)disulfanyl)-2-methylpropyl)nicotinamide (I-1);

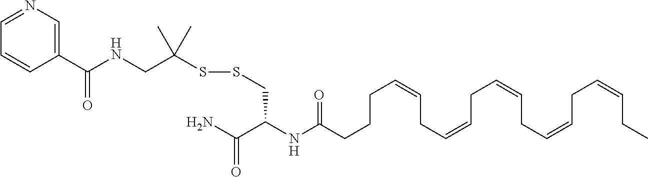 [SMILES: CC/C=C\C/C=C\C/C=C\C/C=C\C/C=C\CCCC(=O)N[C@@H](CSSC(C)(C)CNC(=O)c1cccnc1)C(N)=O]

N-(2-(((R)-3-amino-2-((5Z,8Z,11Z,14Z,17Z)-icosa-5,8,11,14,17-pentaenamido)-3-oxopropyl)disulfanyl)-2-methylpropyl)nicotinamide (I-2);

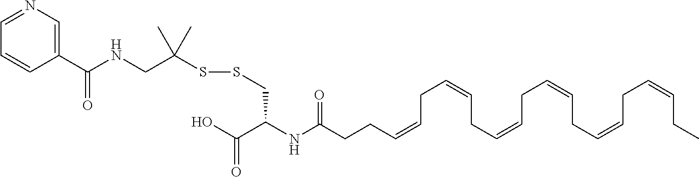 [SMILES: CC/C=C\C/C=C\C/C=C\C/C=C\C/C=C\C/C=C\CCC(=O)N[C@@H](CSSC(C)(C)CNC(=O)c1cccnc1)C(=O)O]

N-((4Z,7Z,10Z,13Z,16Z,19Z)-docosa-4,7,10,13,16,19-hexaenoyl)-S-((2-methyl-1-(nicotinamido)propan-2-yl)thio)-L-cysteine (I-3);

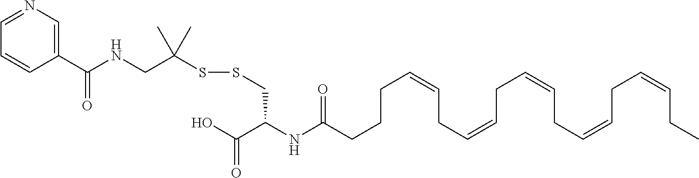 [SMILES: CC/C=C\C/C=C\C/C=C\C/C=C\C/C=C\CCCC(=O)N[C@@H](CSSC(C)(C)CNC(=O)c1cccnc1)C(=O)O]

N-((5Z,8Z,11Z,14Z,17Z)-icosa-5,8,11,14,17-pentaenoyl)-S-((2-methyl-1-(nicotinamido)propan-2-yl)thio)-L-cysteine (I-4);

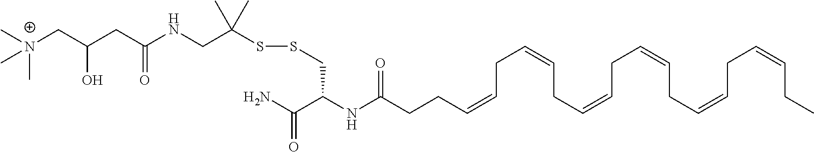 [SMILES: CC/C=C\C/C=C\C/C=C\C/C=C\C/C=C\C/C=C\CCC(=O)N[C@@H](CSSC(C)(C)CNC(=O)CC(O)C[N+](C)(C)C)C(N)=O]

4-((2-(((R)-3-amino-2-((4Z,7Z,10Z,13Z,16Z,19Z)-docosa-4,7,10,13,16,19-hexaenamido)-3-oxopropyl)disulfanyl)-2-methylpropyl)amino)-2-hydroxy-N,N,N-trimethyl-4-oxobutan-1-aminium (I-5);

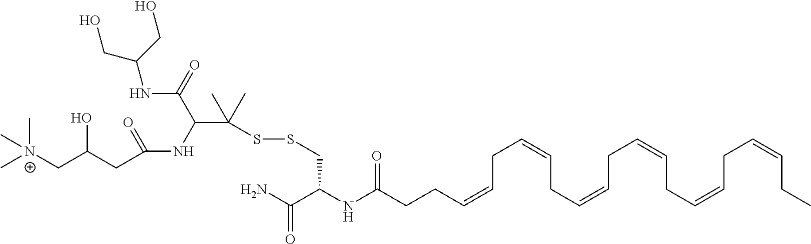 [SMILES: CC/C=C\C/C=C\C/C=C\C/C=C\C/C=C\C/C=C\CCC(=O)N[C@@H](CSSC(C)(C)C(NC(=O)CC(O)C[N+](C)(C)C)C(=O)NC(CO)CO)C(N)=O]

4-((3-(((R)-3-amino-2-((4Z,7Z,10Z,13Z,16Z,19Z)-docosa-4,7,10,13,16,19-hexaenamido)-3-oxopropyl)disulfanyl)-1-((1,3-dihydroxypropan-2-yl)amino)-3-methyl-1-oxobutan-2-yl)amino)-2-hydroxy-N,N,N-trimethyl-4-oxobutan-1-aminium (I-6);

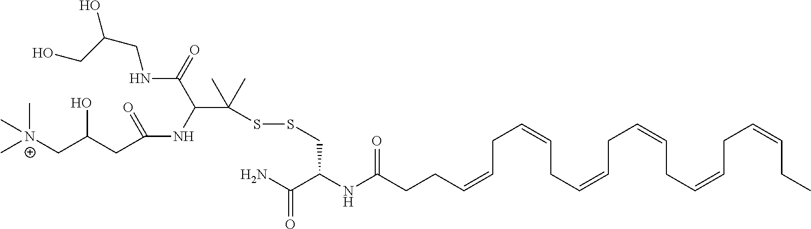 [SMILES: CC/C=C\C/C=C\C/C=C\C/C=C\C/C=C\C/C=C\CCC(=O)N[C@@H](CSSC(C)(C)C(NC(=O)CC(O)C[N+](C)(C)C)C(=O)NCC(O)CO)C(N)=O]

4-((3-(((R)-3-amino-2-((4Z,7Z,10Z,13Z,16Z,19Z)-docosa-4,7,10,13,16,19-hexaenamido)-3-oxopropyl)disulfanyl)-1-((2,3-dihydroxypropyl)amino)-3-methyl-1-oxobutan-2-yl)amino)-2-hydroxy-N,N,N-trimethyl-4-oxobutan-1-aminium (I-7);

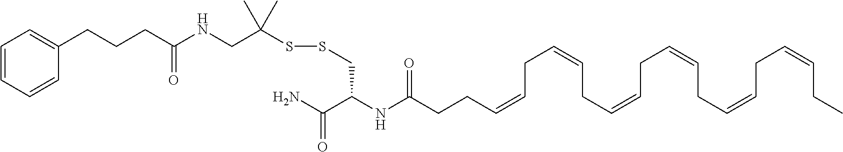 [SMILES: CC/C=C\C/C=C\C/C=C\C/C=C\C/C=C\C/C=C\CCC(=O)N[C@@H](CSSC(C)(C)CNC(=O)CCCc1ccccc1)C(N)=O]

(4Z,7Z,10Z,13Z,16Z,19Z)—N—((R)-1-amino-3-((2-methyl-1-(4-phenylbutanamido)propan-2-yl)disulfanyl)-1-oxopropan-2-yl)docosa-4,7,10,13,16,19-hexaenamide (I-8);

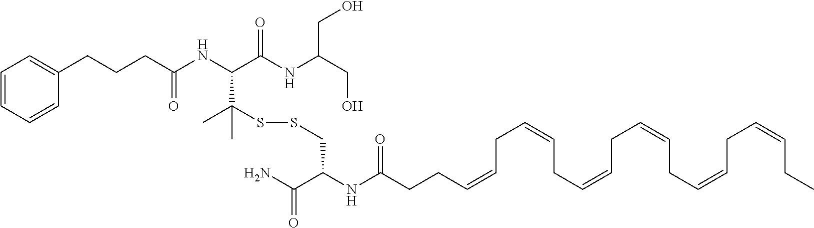 [SMILES: CC/C=C\C/C=C\C/C=C\C/C=C\C/C=C\C/C=C\CCC(=O)N[C@@H](CSSC(C)(C)[C@H](NC(=O)CCCc1ccccc1)C(=O)NC(CO)CO)C(N)=O]

(4Z,7Z,10Z,13Z,16Z,19Z)—N—((R)-1-amino-3-(((R)-4-((1,3-dihydroxypropan-2-yl)amino)-2-methyl-4-oxo-3-(4-phenylbutanamido)butan-2-yl)disulfanyl)-1-oxopropan-2-yl)docosa-4,7,10,13,16,19-hexaenamide (I-9);

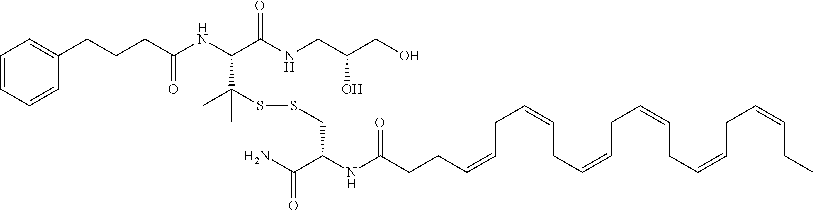 [SMILES: CC/C=C\C/C=C\C/C=C\C/C=C\C/C=C\C/C=C\CCC(=O)N[C@@H](CSSC(C)(C)[C@H](NC(=O)CCCc1ccccc1)C(=O)NC[C@@H](O)CO)C(N)=O]

(4Z,7Z,10Z,13Z,16Z,19Z)—N—((R)-1-amino-3-(((R)-4-(((R)-2,3-dihydroxypropyl)amino)-2-methyl-4-oxo-3-(4-phenylbutanamido)butan-2-yl)disulfanyl)-1-oxopropan-2-yl)docosa-4,7,10,13,16,19-hexaenamide (I-10);

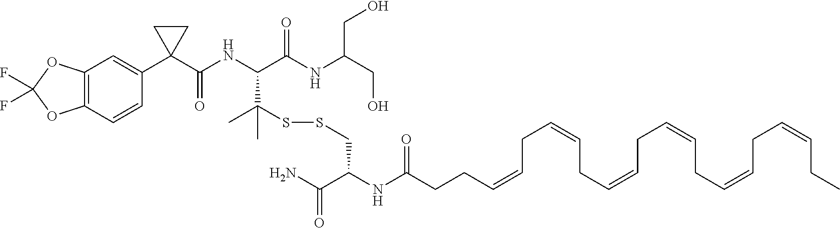 [SMILES: CC/C=C\C/C=C\C/C=C\C/C=C\C/C=C\C/C=C\CCC(=O)N[C@@H](CSSC(C)(C)[C@H](NC(=O)C1(c2ccc3c(c2)OC(F)(F)O3)CC1)C(=O)NC(CO)CO)C(N)=O]

N—((R)-3-(((R)-3-amino-2-((4Z,7Z,10Z,13Z,16Z,19Z)-docosa-4,7,10,13,16,19-hexaenamido)-3-oxopropyl)disulfanyl)-1-((1,3-dihydroxypropan-2-yl)amino)-3-methyl-1-oxobutan-2-yl)-1-(2,2-difluorobenzo[d][1,3]dioxol-5-yl)cyclopropane-1-carboxamide (I-11);

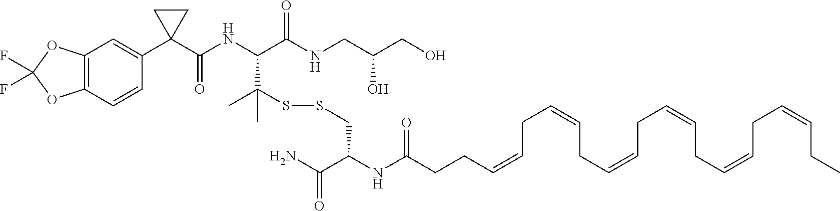 [SMILES: CC/C=C\C/C=C\C/C=C\C/C=C\C/C=C\C/C=C\CCC(=O)N[C@@H](CSSC(C)(C)[C@H](NC(=O)C1(c2ccc3c(c2)OC(F)(F)O3)CC1)C(=O)NC[C@@H](O)CO)C(N)=O]

N—((R)-3-(((R)-3-amino-2-((4Z,7Z,10Z,13Z,16Z,19Z)-docosa-4,7,10,13,16,19-hexaenamido)-3-oxopropyl)disulfanyl)-1-(((R)-2,3-dihydroxypropyl)amino)-3-methyl-1-oxobutan-2-yl)-1-(2,2-difluorobenzo[d][1,3]dioxol-5-yl)cyclopropane-1-carboxamide (I-12);

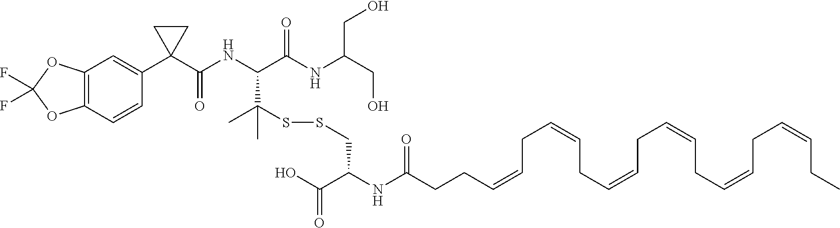 [SMILES: CC/C=C\C/C=C\C/C=C\C/C=C\C/C=C\C/C=C\CCC(=O)N[C@@H](CSSC(C)(C)[C@H](NC(=O)C1(c2ccc3c(c2)OC(F)(F)O3)CC1)C(=O)NC(CO)CO)C(=O)O]

S—(((R)-3-(1-(2,2-difluorobenzo[d][1,3]dioxol-5-yl)cyclopropane-1-carboxamido)-4-((1,3-dihydroxypropan-2-yl)amino)-2-methyl-4-oxobutan-2-yl)thio)-N-((4Z,7Z,10Z,13Z,16Z,19Z)-docosa-4,7,10,13,16,19-hexaenoyl)-L-cysteine (I-13);

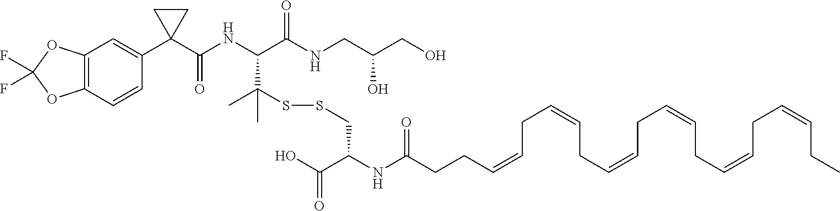 [SMILES: CC/C=C\C/C=C\C/C=C\C/C=C\C/C=C\C/C=C\CCC(=O)N[C@@H](CSSC(C)(C)[C@H](NC(=O)C1(c2ccc3c(c2)OC(F)(F)O3)CC1)C(=O)NC[C@@H](O)CO)C(=O)O]

S—(((R)-3-(1-(2,2-difluorobenzo[d][1,3]dioxol-5-yl)cyclopropane-1-carboxamido)-4-(((R)-2,3-dihydroxypropyl)amino)-2-methyl-4-oxobutan-2-yl)thio)-N-((4Z,7Z,10Z,13Z,16Z,19Z)-docosa-4,7,10,13,16,19-hexaenoyl)-L-cysteine (I-14);

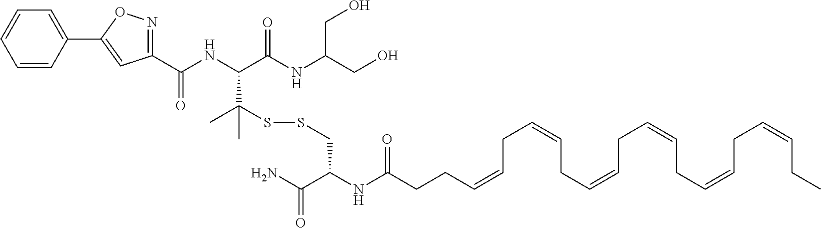 [SMILES: CC/C=C\C/C=C\C/C=C\C/C=C\C/C=C\C/C=C\CCC(=O)N[C@@H](CSSC(C)(C)[C@H](NC(=O)c1cc(-c2ccccc2)on1)C(=O)NC(CO)CO)C(N)=O]

N—((R)-3-(((R)-3-amino-2-((4Z,7Z,10Z,13Z,16Z,19Z)-docosa-4,7,10,13,16,19-hexaenamido)-3-oxopropyl)disulfanyl)-1-((1,3-dihydroxypropan-2-yl)amino)-3-methyl-1-oxobutan-2-yl)-5-phenylisoxazole-3-carboxamide (I-15);

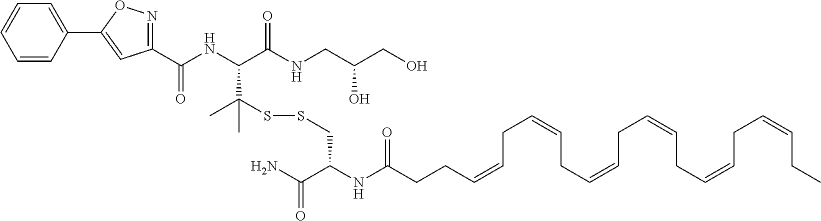 [SMILES: CC/C=C\C/C=C\C/C=C\C/C=C\C/C=C\C/C=C\CCC(=O)N[C@@H](CSSC(C)(C)[C@H](NC(=O)c1cc(-c2ccccc2)on1)C(=O)NC[C@@H](O)CO)C(N)=O]

N—((R)-3-(((R)-3-amino-2-((4Z,7Z,10Z,13Z,16Z,19Z)-docosa-4,7,10,13,16,19-hexaenamido)-3-oxopropyl)disulfanyl)-1-(((R)-2,3-dihydroxypropyl)amino)-3-methyl-1-oxobutan-2-yl)-5-phenylisoxazole-3-carboxamide (I-16);

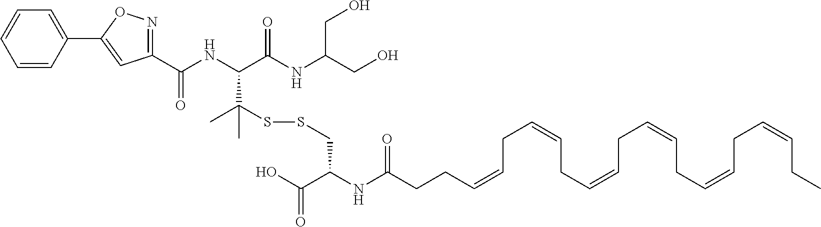 [SMILES: CC/C=C\C/C=C\C/C=C\C/C=C\C/C=C\C/C=C\CCC(=O)N[C@@H](CSSC(C)(C)[C@H](NC(=O)c1cc(-c2ccccc2)on1)C(=O)NC(CO)CO)C(=O)O]

S—(((R)-4-((1,3-dihydroxypropan-2-yl)amino)-2-methyl-4-oxo-3-(5-phenylisoxazole-3-carboxamido)butan-2-yl)thio)-N-((4Z,7Z,10Z,13Z,16Z,19Z)-docosa-4,7,10,13,16,19-hexaenoyl)-L-cysteine (I-17);

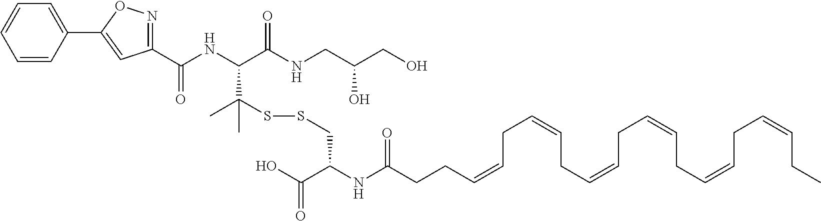 [SMILES: CC/C=C\C/C=C\C/C=C\C/C=C\C/C=C\C/C=C\CCC(=O)N[C@@H](CSSC(C)(C)[C@H](NC(=O)c1cc(-c2ccccc2)on1)C(=O)NC[C@@H](O)CO)C(=O)O]

S—(((R)-4-(((R)-2,3-dihydroxypropyl)amino)-2-methyl-4-oxo-3-(5-phenylisoxazole-3-carboxamido)butan-2-yl)thio)-N-((4Z,7Z,10Z,13Z,16Z,19Z)-docosa-4,7,10,13,16,19-hexaenoyl)-L-cysteine (I-18);

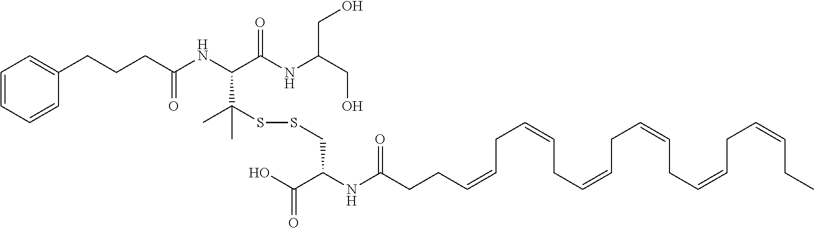 [SMILES: CC/C=C\C/C=C\C/C=C\C/C=C\C/C=C\C/C=C\CCC(=O)N[C@@H](CSSC(C)(C)[C@H](NC(=O)CCCc1ccccc1)C(=O)NC(CO)CO)C(=O)O]

S—(((R)-4-((1,3-dihydroxypropan-2-yl)amino)-2-methyl-4-oxo-3-(4-phenylbutanamido)butan-2-yl)thio)-N-((4Z,7Z,10Z,13Z,16Z,19Z)-docosa-4,7,10,13,16,19-hexaenoyl)-L-cysteine (I-19);

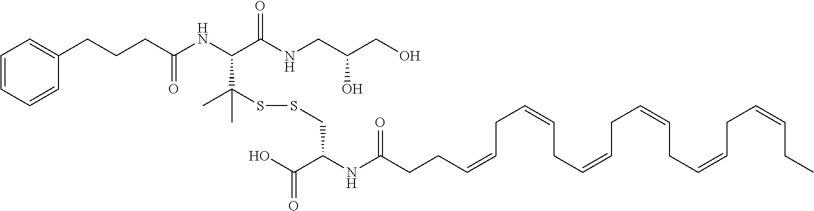 [SMILES: CC/C=C\C/C=C\C/C=C\C/C=C\C/C=C\C/C=C\CCC(=O)N[C@@H](CSSC(C)(C)[C@H](NC(=O)CCCc1ccccc1)C(=O)NC[C@@H](O)CO)C(=O)O]

S—(((R)-4-(((R)-2,3-dihydroxypropyl)amino)-2-methyl-4-oxo-3-(4-phenylbutanamido)butan-2-yl)thio)-N-((4Z,7Z,10Z,13Z,16Z,19Z)-docosa-4,7,10,13,16,19-hexaenoyl)-L-cysteine (I-20);

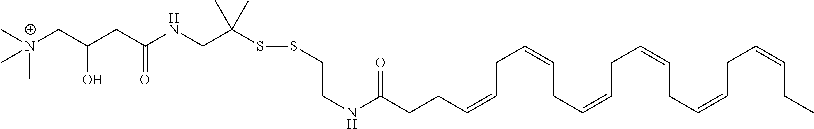 [SMILES: CC/C=C\C/C=C\C/C=C\C/C=C\C/C=C\C/C=C\CCC(=O)NCCSSC(C)(C)CNC(=O)CC(O)C[N+](C)(C)C]

4-((2-((2-((4Z,7Z,10Z,13Z,16Z,19Z)-docosa-4,7,10,13,16,19-hexaenamido)ethyl)disulfanyl)-2-methylpropyl)amino)-2-hydroxy-N,N,N-trimethyl-4-oxobutan-1-aminium (III-1);

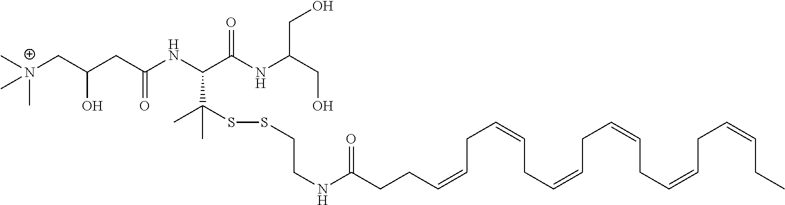 [SMILES: CC/C=C\C/C=C\C/C=C\C/C=C\C/C=C\C/C=C\CCC(=O)NCCSSC(C)(C)[C@H](NC(=O)CC(O)C[N+](C)(C)C)C(=O)NC(CO)CO]

4-(((R)-1-((1,3-dihydroxypropan-2-yl)amino)-3-((2-((4Z,7Z,10Z,13Z,16Z,19Z)-docosa-4,7,10,13,16,19-hexaenamido)ethyl)disulfanyl)-3-methyl-1-oxobutan-2-yl)amino)-2-hydroxy-N,N,N-trimethyl-4-oxobutan-1-aminium (III-2);

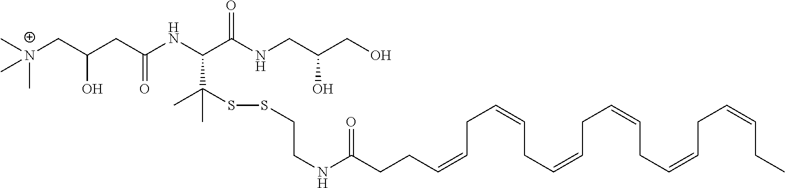 [SMILES: CC/C=C\C/C=C\C/C=C\C/C=C\C/C=C\C/C=C\CCC(=O)NCCSSC(C)(C)[C@H](NC(=O)CC(O)C[N+](C)(C)C)C(=O)NC[C@@H](O)CO]

4-(((R)-1-(((R)-2,3-dihydroxypropyl)amino)-3-((2-((4Z,7Z,10Z,13Z,16Z,19Z)-docosa-4,7,10,13,16,19-hexaenamido)ethyl)disulfanyl)-3-methyl-1-oxobutan-2-yl)amino)-2-hydroxy-N,N,N-trimethyl-4-oxobutan-1-aminium (III-3);

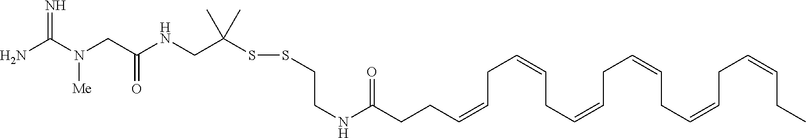 [SMILES: CC/C=C\C/C=C\C/C=C\C/C=C\C/C=C\C/C=C\CCC(=O)NCCSSC(C)(C)CNC(=O)CN(C)C(=N)N]

(4Z,7Z,10Z,13Z,16Z,19Z)—N-(2-((2-methyl-1-(2-(1-methylguanidino)acetamido)propan-2-yl)disulfanyl)ethyl)docosa-4,7,10,13,16,19-hexaenamide (III-4);

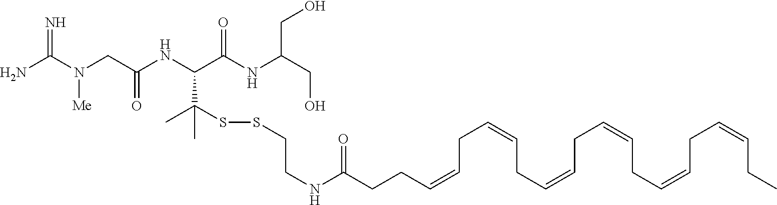 [SMILES: CC/C=C\C/C=C\C/C=C\C/C=C\C/C=C\C/C=C\CCC(=O)NCCSSC(C)(C)[C@H](NC(=O)CN(C)C(=N)N)C(=O)NC(CO)CO]

(4Z,7Z,10Z,13Z,16Z,19Z)—N-(2-(((R)-4-((1,3-dihydroxypropan-2-yl)amino)-2-methyl-3-(2-(1-methylguanidino)acetamido)-4-oxobutan-2-yl)disulfanyl)ethyl)docosa-4,7,10,13,16,19-hexaenamide (III-5);

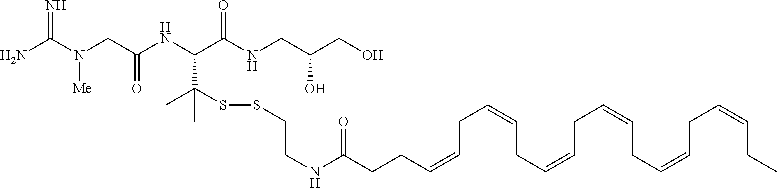 [SMILES: CC/C=C\C/C=C\C/C=C\C/C=C\C/C=C\C/C=C\CCC(=O)NCCSSC(C)(C)[C@H](NC(=O)CN(C)C(=N)N)C(=O)NC[C@@H](O)CO]

(4Z,7Z,10Z,13Z,16Z,19Z)—N-(2-(((R)-4-(((R)-2,3-dihydroxypropyl)amino)-2-methyl-3-(2-(1-methylguanidino)acetamido)-4-oxobutan-2-yl)disulfanyl)ethyl)docosa-4,7,10,13,16,19-hexaenamide (III-6);

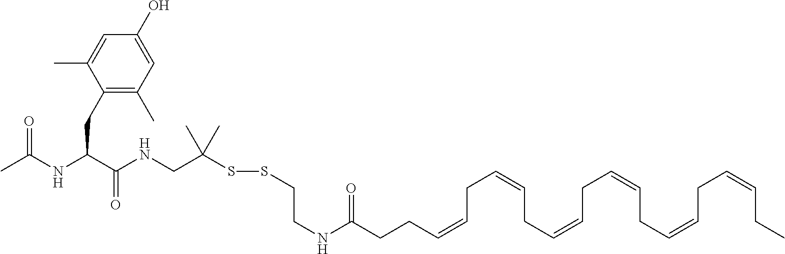 [SMILES: CC/C=C\C/C=C\C/C=C\C/C=C\C/C=C\C/C=C\CCC(=O)NCCSSC(C)(C)CNC(=O)[C@H](Cc1c(C)cc(O)cc1C)NC(C)=O]

(4Z,7Z,10Z,13Z,16Z,19Z)—N-(2-((1-((S)-2-acetamido-3-(4-hydroxy-2,6-dimethylphenyl)propanamido)-2-methylpropan-2-yl)disulfanyl)ethyl)docosa-4,7,10,13,16,19-hexaenamide (III-7);

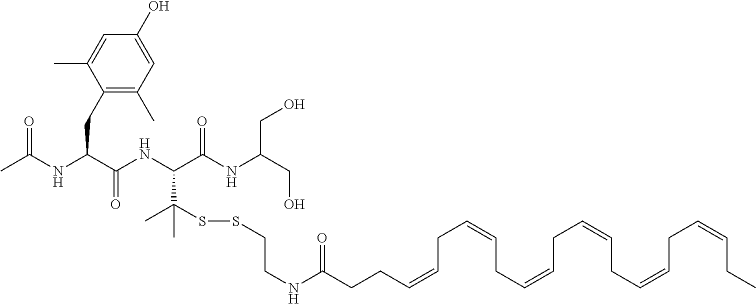 [SMILES: CC/C=C\C/C=C\C/C=C\C/C=C\C/C=C\C/C=C\CCC(=O)NCCSSC(C)(C)[C@H](NC(=O)[C@H](Cc1c(C)cc(O)cc1C)NC(C)=O)C(=O)NC(CO)CO]

(4Z,7Z,10Z,13Z,16Z,19Z)—N-(2-(((R)-3-((S)-2-acetamido-3-(4-hydroxy-2,6-dimethylphenyl)propanamido)-4-((1,3-dihydroxypropan-2-yl)amino)-2-methyl-4-oxobutan-2-yl)disulfanyl)ethyl)docosa-4,7,10,13,16,19-hexaenamide (III-8);

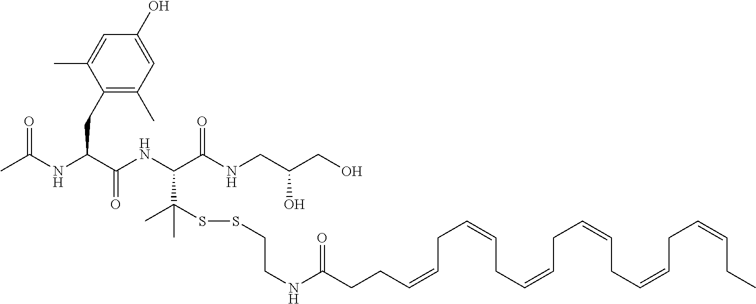 [SMILES: CC/C=C\C/C=C\C/C=C\C/C=C\C/C=C\C/C=C\CCC(=O)NCCSSC(C)(C)[C@H](NC(=O)[C@H](Cc1c(C)cc(O)cc1C)NC(C)=O)C(=O)NC[C@@H](O)CO]

(4Z,7Z,10Z,13Z,16Z,19Z)—N-(2-(((R)-3-((S)-2-acetamido-3-(4-hydroxy-2,6-dimethylphenyl)propanamido)-4-(((R)-2,3-dihydroxypropyl)amino)-2-methyl-4-oxobutan-2-yl)disulfanyl)ethyl)docosa-4,7,10,13,16,19-hexaenamide (III-9);

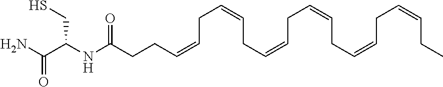 [SMILES: CC/C=C\C/C=C\C/C=C\C/C=C\C/C=C\C/C=C\CCC(=O)N[C@@H](CS)C(N)=O]

(4Z,7Z,10Z,13Z,16Z,19Z)—N—((R)-1-amino-3-mercapto-1-oxopropan-2-yl)docosa-4,7,10,13,16,19-hexaenamide (IV-1);

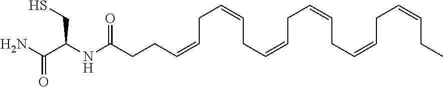 [SMILES: CC/C=C\C/C=C\C/C=C\C/C=C\C/C=C\C/C=C\CCC(=O)N[C@H](CS)C(N)=O]

(4Z,7Z,10Z,13Z,16Z,19Z)—N—((S)-1-amino-3-mercapto-1-oxopropan-2-yl)docosa-4,7,10,13,16,19-hexaenamide (IV-2);

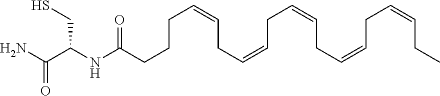 [SMILES: CC/C=C\C/C=C\C/C=C\C/C=C\C/C=C\CCCC(=O)N[C@@H](CS)C(N)=O]

(5Z,8Z,11Z,14Z,17Z)—N—((R)-1-amino-3-mercapto-1-oxopropan-2-yl)icosa-5,8,11,14,17-pentaenamide (IV-3); and

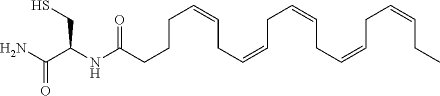 [SMILES: CC/C=C\C/C=C\C/C=C\C/C=C\C/C=C\CCCC(=O)N[C@H](CS)C(N)=O]

(5Z,8Z,11Z,14Z,17Z)—N—((S)-1-amino-3-mercapto-1-oxopropan-2-yl)icosa-5,8,11,14,17-pentaenamide (IV-4).

The invention also provides any of the foregoing compounds as a pharmaceutically acceptable salt thereof.

In certain embodiments, the compound is:

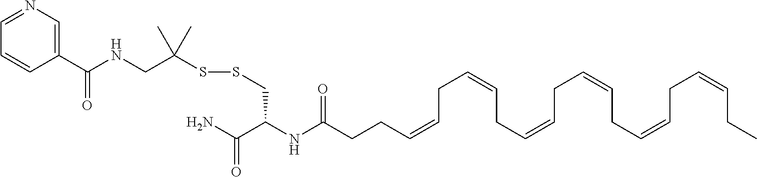 [SMILES: CC/C=C\C/C=C\C/C=C\C/C=C\C/C=C\C/C=C\CCC(=O)N[C@@H](CSSC(C)(C)CNC(=O)c1cccnc1)C(N)=O]

N-(2-(((R)-3-amino-2-((4Z,7Z,10Z,13Z,16Z,19Z)-docosa-4,7,10,13,16,19-hexaenamido)-3-oxopropyl)disulfanyl)-2-methylpropyl)nicotinamide (I-1) or a pharmaceutically acceptable salt thereof.

In certain embodiments, the compound is:

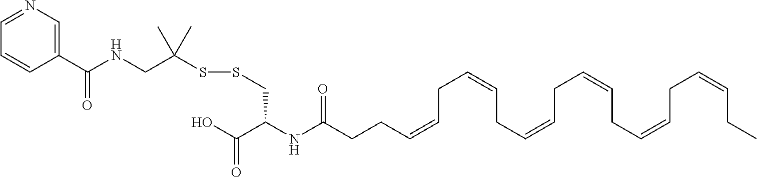 [SMILES: CC/C=C\C/C=C\C/C=C\C/C=C\C/C=C\C/C=C\CCC(=O)N[C@@H](CSSC(C)(C)CNC(=O)c1cccnc1)C(=O)O]

N-((4Z,7Z,10Z,13Z,16Z,19Z)-docosa-4,7,10,13,16,19-hexaenoyl)-S-((2-methyl-1-(nicotinamido)propan-2-yl)thio)-L-cysteine (I-3) or a pharmaceutically acceptable salt thereof.

In certain embodiments, the compound is:

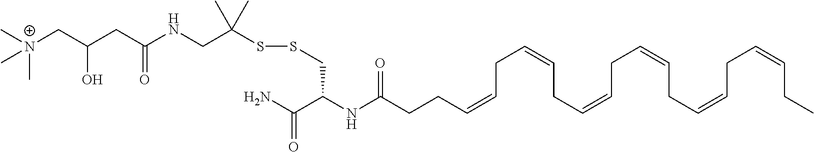 [SMILES: CC/C=C\C/C=C\C/C=C\C/C=C\C/C=C\C/C=C\CCC(=O)N[C@@H](CSSC(C)(C)CNC(=O)CC(O)C[N+](C)(C)C)C(N)=O]

4-((2-(((R)-3-amino-2-((4Z,7Z,10Z,13Z,16Z,19Z)-docosa-4,7,10,13,16,19-hexaenamido)-3-oxopropyl)disulfanyl)-2-methylpropyl)amino)-2-hydroxy-N,N,N-trimethyl-4-oxobutan-1-aminium (I-5) or a pharmaceutically acceptable salt thereof.

In certain embodiments, the compound is:

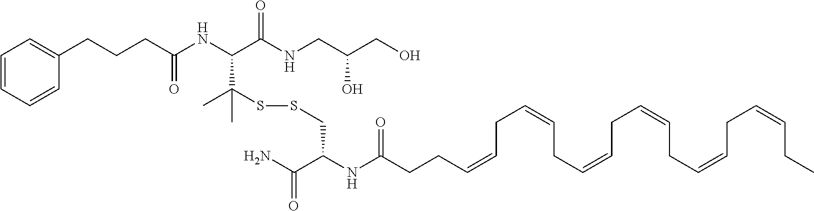 [SMILES: CC/C=C\C/C=C\C/C=C\C/C=C\C/C=C\C/C=C\CCC(=O)N[C@@H](CSSC(C)(C)[C@H](NC(=O)CCCc1ccccc1)C(=O)NC[C@@H](O)CO)C(N)=O]

(4Z,7Z,10Z,13Z,16Z,19Z)—N—((R)-1-amino-3-(((R)-4-(((R)-2,3-dihydroxypropyl)amino)-2-methyl-4-oxo-3-(4-phenylbutanamido)butan-2-yl)disulfanyl)-1-oxopropan-2-yl)docosa-4,7,10,13,16,19-hexaenamide (I-10) or a pharmaceutically acceptable salt thereof.

In certain embodiments, the compound is:

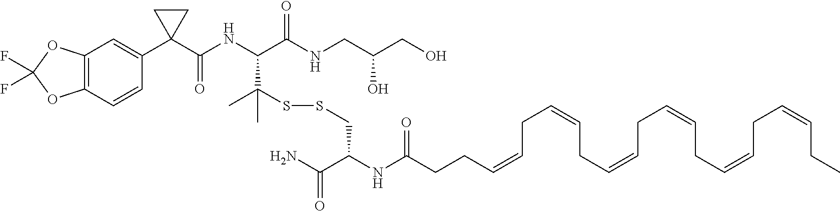 [SMILES: CC/C=C\C/C=C\C/C=C\C/C=C\C/C=C\C/C=C\CCC(=O)N[C@@H](CSSC(C)(C)[C@H](NC(=O)C1(c2ccc3c(c2)OC(F)(F)O3)CC1)C(=O)NC[C@@H](O)CO)C(N)=O]

N—((R)-3-(((R)-3-amino-2-((4Z,7Z,10Z,13Z,16Z,19Z)-docosa-4,7,10,13,16,19-hexaenamido)-3-oxopropyl)disulfanyl)-1-(((R)-2,3-dihydroxypropyl)amino)-3-methyl-1-oxobutan-2-yl)-1-(2,2-difluorobenzo[d][1,3]dioxol-5-yl)cyclopropane-1-carboxamide (I-12) or a pharmaceutically acceptable salt thereof.

In certain embodiments, the compound is:

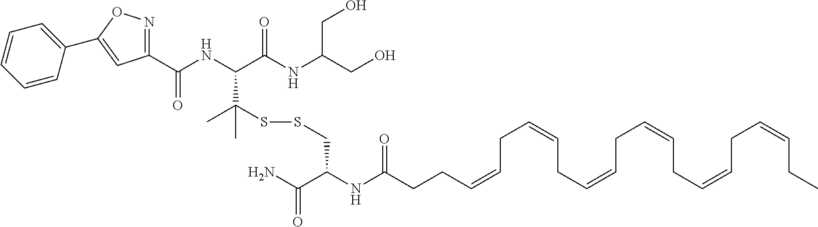 [SMILES: CC/C=C\C/C=C\C/C=C\C/C=C\C/C=C\C/C=C\CCC(=O)N[C@@H](CSSC(C)(C)[C@H](NC(=O)c1cc(-c2ccccc2)on1)C(=O)NC(CO)CO)C(N)=O]

N—((R)-3-(((R)-3-amino-2-((4Z,7Z,10Z,13Z,16Z,19Z)-docosa-4,7,10,13,16,19-hexaenamido)-3-oxopropyl)disulfanyl)-1-((1,3-dihydroxypropan-2-yl)amino)-3-methyl-1-oxobutan-2-yl)-5-phenylisoxazole-3-carboxamide (I-15) or a pharmaceutically acceptable salt thereof.

In certain embodiments, the compound is:

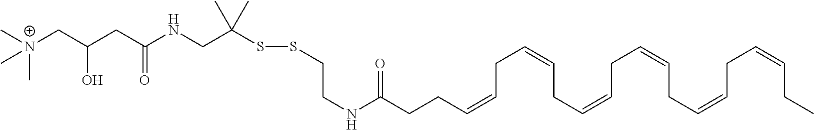 [SMILES: CC/C=C\C/C=C\C/C=C\C/C=C\C/C=C\C/C=C\CCC(=O)NCCSSC(C)(C)CNC(=O)CC(O)C[N+](C)(C)C]

4-((2-((2-((4Z,7Z,10Z,13Z,16Z,19Z)-docosa-4,7,10,13,16,19-hexaenamido)ethyl)disulfanyl)-2-methylpropyl)amino)-2-hydroxy-N,N,N-trimethyl-4-oxobutan-1-aminium (III-1) or a pharmaceutically acceptable salt thereof.

In certain embodiments, the compound is:

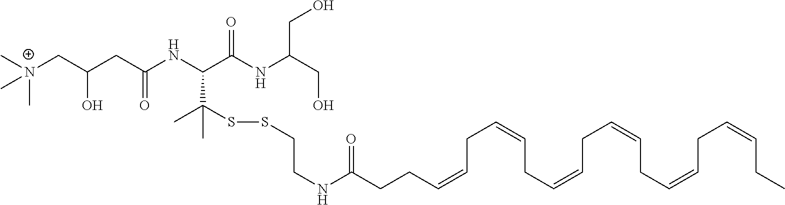 [SMILES: CC/C=C\C/C=C\C/C=C\C/C=C\C/C=C\C/C=C\CCC(=O)NCCSSC(C)(C)[C@H](NC(=O)CC(O)C[N+](C)(C)C)C(=O)NC(CO)CO]

4-(((R)-1-((1,3-dihydroxypropan-2-yl)amino)-3-((2-((4Z,7Z,10Z,13Z,16Z,19Z)-docosa-4,7,10,13,16,19-hexaenamido)ethyl)disulfanyl)-3-methyl-1-oxobutan-2-yl)amino)-2-hydroxy-N,N,N-trimethyl-4-oxobutan-1-aminium (III-2) or a pharmaceutically acceptable salt thereof.

In certain embodiments, the compound is the following or a pharmaceutically acceptable salt thereof:

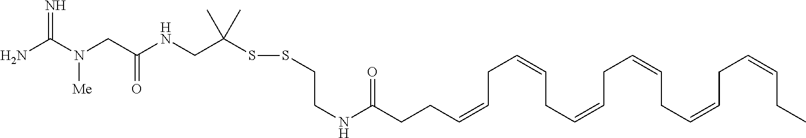 [SMILES: CC/C=C\C/C=C\C/C=C\C/C=C\C/C=C\C/C=C\CCC(=O)NCCSSC(C)(C)CNC(=O)CN(C)C(=N)N]

(4Z,7Z,10Z,13Z,16Z,19Z)—N-(2-((2-methyl-1-(2-(1-methylguanidino)acetamido)propan-2-yl)disulfanyl)ethyl)docosa-4,7,10,13,16,19-hexaenamide (III-4) or a pharmaceutically acceptable salt thereof.

In certain embodiments, the compound is:

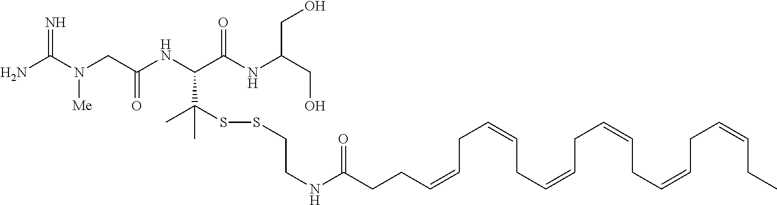 [SMILES: CC/C=C\C/C=C\C/C=C\C/C=C\C/C=C\C/C=C\CCC(=O)NCCSSC(C)(C)[C@H](NC(=O)CN(C)C(=N)N)C(=O)NC(CO)CO]

(4Z,7Z,10Z,13Z,16Z,19Z)—N-(2-(((R)-4-((1,3-dihydroxypropan-2-yl)amino)-2-methyl-3-(2-(1-methylguanidino)acetamido)-4-oxobutan-2-yl)disulfanyl)ethyl)docosa-4,7,10,13,16,19-hexaenamide (III-5) or a pharmaceutically acceptable salt thereof.

As indicated above, the invention provides a pharmaceutical composition comprising a compound described herein, for example, a compound of Formula I, Formula II, Formula III or Formula IV or a pharmaceutically acceptable salt thereof, and a pharmaceutically acceptable carrier. It is contemplated that the compounds of the invention can be synthesized using conventional reagents and methodologies, for example, by using exemplary reagents and exemplary methods and protocols set forth in the Examples.

### III. Therapeutic Applications of Fatty Acid Cysteine-Based Conjugates

As indicated above, the invention is based, in part, upon the discovery that fatty acid cysteine-based conjugates are useful in activating autophagy. In addition, the conjugates of the invention are useful in improving mitochondrial bioenergetics.

The conjugates of the invention have therapeutic effects that cannot be achieved by administering the individual components, for example, the three individual components, separately or as a combination of individual components, and offer a superior way of activating autophagy to treat CF in a way that cannot be replicated by administering the individual components or the combination of the individual components. The covalent linkage of cysteine and an omega-3 fatty acid allows the simultaneous delivery of the two components to an intracellular location, whereupon the individual components are released by cleavage, for example, enzymatic cleavage, at the location and at the same time. Exemplary therapeutic methods and additional features of the therapeutic applications are described below.

### Exemplary Therapeutic Methods

One aspect of the invention provides a method of treating a disease described herein (e.g., a disease selected from the group consisting of CF, a neurodegenerative disease, inflammatory disease, a liver disease, muscle disease, infection, and an immune disease). The method comprises administering to a patient in need thereof a therapeutically effective amount of a fatty acid cysteamine conjugate of a CFTR modulator described herein, such as a compound of Formula I, II, III, or IV to treat the disease. In certain embodiments, the compound is a compound of Formula I or a salt thereof. In certain embodiments, the compound is a compound of Formula II or a salt thereof. In certain embodiments, the compound is a compound of Formula III or a salt thereof. In certain embodiments, the compound is a compound of Formula IV or a salt thereof.

In certain embodiments, the disease a neurodegenerative disease, liver disease, muscle disease, infection, immunity, or inflammatory disease. In certain embodiments, the disease is CF. In certain embodiments, the disease is a neurodegenerative disease (e.g., Huntington's disease, Alzheimer's disease, Parkinson's disease, or transmissible spongiform encephalopathies). In certain embodiments, the disease is an inflammatory disease. In certain embodiments, the disease is idiopathic pulmonary fibrosis. In certain embodiments, the disease is age-related macular degeneration. In yet other embodiments, the disease is a cardiac disease.

In certain embodiments, in the method of treating the disease, the administration of the compound of Formula I increases autophagy in a subject by at least 5%, 10%, 25%, 50%, or 100%. In certain embodiments, in the method of treating the disease, the administration of the compound of Formula II increases autophagy in a subject by at least 5%, 10%, 25%, 50%, or 100%. In certain embodiments, in the method of treating the disease, the administration of the compound of Formula III increases autophagy in a subject by at least 5%, 10%, 25%, 50%, or 100%. In certain embodiments, in the method of treating the disease, the administration of the compound of Formula IV increases autophagy in a subject by at least 5%, 10%, 25%, 50%, or 100%.

Additional diseases contemplated for treatment using methods described herein include, for example, the following diseases that are understood to have defective autophagy: Danon disease, X-linked myopathy, infantile autophagic vacuolar myopathy, adult onset vacuolar myopathy, Pompe disease, sporadic inclusion body myositis, limb girdle muscular dystrophy type 2B, and Miyoshi myopathy.

The fatty acid cysteine-based conjugates described herein may also useful for the treatment of mitochondrial diseases such as Leigh Syndrome, Diabetes Mellitus and Deafness (DAD), Leber's hereditary optic neuropathy, Neuropathy-ataxia-retinis pigmentosa and ptosis (NARP), myoneurogenic gastrointestinal encephalopathy (MNGIE), myoclonic epilepsy with ragged red fibers (MERRF), and mitochondrial myopathy-encephalomyopathy-lactic acidosis-stroke like symptoms (MELAS), Keam-Sayre syndrome, subacute necrotizing encephalopathy (Leigh's Syndrome), and mitochondrial cardiomyopathies and othersyndromes due to multiplemitochondrial DNA deletions. Additional mitochondrial diseases include neurogenic muscle weakness, progressive external opthalmoplegia (PEO), and Complex I disease, Complex II disease, Complex III disease, Complex IV disease and Complex V disease, which relates to dysfunction of the OXPHOS complexes, and MEGDEL syndrome (3-methylglutaconic aciduria type IV with sensorineural deafness, encephalopathy and Leigh-like syndrome.

In certain embodiments, the patient is a human.

Another aspect of the invention provides a method of activating autophagy in a patient. The method comprises administering to a patient in need thereof an effective amount of a fatty acid cysteine-based conjugates described herein, such as a compound of Formula I, II, II, or IV, to activate autophagy in the patient. In certain embodiments, the patient suffers from CF, a neurodegenerative disease, or an inflammatory disease.

Another aspect of the invention provides a method of improving mitochondrial bioenergetics in a patient. The method comprises administering to a patient in need thereof an effective amount of a fatty acid cysteine-based conjugates described herein, such as a compound of Formula I, II, III, or IV or a salt of any of the foregoing, to improve mitochondrial bioenergetics in the patient. In certain embodiments, the patient suffers from CF, a neurodegenerative disease, or an inflammatory disease.

In certain embodiments, in the method of treating the disease, the administration of a compound for example, Formula I increases autophagy in a subject by at least 5%, 10%, 25%, 50% or least 100%. In certain embodiments, in the method of treating the disease, the administration of a compound for example, Formula II increases autophagy in a subject by at least 5%, 10%, 25%, 50% or least 100%. In certain embodiments, in the method of treating the disease, the administration of a compound for example, Formula III increases autophagy in a subject by at least 5%, 10%, 25%, 50% or least 100%. In certain embodiments, in the method of treating the disease, the administration of a compound for example, Formula IV increases autophagy in a subject by at least 5%, 10%, 25%, 50% or least 100%.

In certain embodiments, activation of autophagy can be characterized according to changes in the amount of certain biomarkers. One exemplary biomarker is microtubule-associated protein 1A/1B-light chain 3 (LC3), which is a soluble protein with a molecular mass of approximately 17 kDa that occurs throughout many mammalian tissues and cultured cells. In cells, a cytosolic form of LC3 (LC3-I) becomes conjugated to phosphatidylethanolamine to form LC3-phosphatidylethanolamine conjugate (LC3-II). See, for example, Tanida et al. (2008) Methods Mol. Biol., vol 445, p. 77-88. The amount of LC3-II relative to LC3-I can be used to analyze changes in the amount of autophagy. Accordingly, in certain embodiments, the administration of one or more of the foregoing compounds increases the ratio of LC3-II to LC3-I in the subject, such as at least about 10%, 25%, 50%, 75%, or 100%.

Another exemplary biomarker is p62 protein, also called sequestosome 1 (SQSTM1), which is an ubiquitin-binding scaffold protein that has been reported to colocalize with ubiquitinated protein aggregates. See, for example, Bjorkoy et al. (2009) METHODS ENZYMOL., vol. 452, p. 181-197. Accordingly, in certain embodiments, the administration of one or more of the foregoing compounds decreases the amount of p62 protein in the subject, such as by at least about 1%, 5%, 10%, 15%, 25%, 50%, 75%, or 90% w/w reduction in the amount of p62 protein in the subject relative to the level prior to treatment with the compound.

In certain embodiments, in the method for increasing autophagy, the subject has been diagnosed as having CF. In certain embodiments, in the method for increasing autophagy, the subject has been diagnosed as having a neurodegenerative disease.

Further, and more generally, another aspect of the invention provides a method of increasing autophagy, wherein the method comprises administering to a subject in need thereof an effective amount of a molecular conjugate comprising a fatty acid covalently linked to cysteine, wherein the fatty acid is selected from the group consisting of omega-3 fatty acids and fatty acids that are metabolized in vivo to omega-3 fatty acids.

Another aspect of the invention provides a method of treating a disease susceptible to treatment with a fatty acid cysteine conjugate in a subject in need thereof by administering to the patient an effective amount of a fatty acid cysteamine conjugate of a CFTR modulator.

Another aspect of the invention provides a method of treating a metabolic disease by administering to a subject in need thereof an effective amount of a fatty acid cysteine conjugate.

### Additional Features of Autophagy, Mitochondrial Bioenergetics, Cysteine, and Conjugates

Autophagy is an evolutionarily conserved lysosomal degradation pathway to essentially self-digest some of the cellular components (see, Levine and Kroemer (2008) CELL, 132, p. 27-42). This self-digestion process serves as a means to help cells remove extraneous or damaged organelles, defective or mis-folded proteins and even invading microorganisms. It is known that autophagy is down-regulated in CF patients (Luciani et al. (2011) AUTOPHAGY, 7, p. 104-106). Autophagy also represents an important cellular mechanism for removing pathogens such as Pseudomonas aegurinosa from infected tissues such as lungs. Activation of autophagy can potentially help CF patients clear out Pseudomonas aegurinosa from their chronically infected lungs (Junkins et al. (2013) PLOS ONE, 8, e72263).

In CF, the defective CFTR causes an up-regulation of reactive oxygen species, which increases the activity of tissue transglutaminase (TG2), an enzyme that facilitates the cross linking between proteins. The increased TG2 activity induces the cross-linking of Beclin-1, a key protein in regulating autophagy. The cross-linking process of Beclin-1 displaces it from the endoplasmic reticulum, down-regulates autophagy and consequently causes an accumulation of p62 (also referred to SQSTM1). The increased p62 can sequester the mis-folded CFTR into aggresomes, which are then targeted for degradation by proteasomes. It has been observed that when human epithelial cells from CF patients with homozygous ΔF508 mutation were treated with a high concentration of cystamine (250 μM), there was an up-regulation of autophagy and a restoration of the CFTR to the plasma membrane (Luciani et al. (2012) AUTOPHAGY, 8, p. 1657-1672; Luciani et al. (2010) NAT. CELL BIOL., 12, p. 863-875). The rationale was that cystamine can inhibit TG2 activity, which decreases the cross-linking of BECN1. This process causes a reduction in the level of p62, which then allows the mis-folded CFTR to escape sequestration into the aggresomes and to localize in the Golgi for transport to the membrane. Though promising, this method of restoring activity to the defective CFTR has one major drawback, namely the high concentration of cystamine that is needed to induce autophagy in various epithelial cell lines (250 μM). It is contemplated that this high concentration of cystamine would require a human dose that may be impractical as well as potentially non-compliant to the patients since it is known that cystamine/cysteamine can induce a significant level of GI discomfort at high doses (Kan et al. (1984) BRIT. J. EXP. PATHOLOGY, 65, p. 759-765).

The fatty acid cysteine-based conjugates have been designed to bring together cysteine and an omega-3 fatty acid into a single molecular conjugate. The activity of the fatty acid cysteine conjugate is substantially greater than the sum of the individual components of the molecular conjugate, suggesting that the activity induced by the fatty acid cysteine conjugate is synergistic. Another benefit of the fatty acid cysteine-based conjugates of the invention is that they demonstrate very low or no peripheral toxicity.

### IV. Pharmaceutical Compositions

The invention provides pharmaceutical compositions comprising a fatty acid cysteine-based conjugate, such as a compound of Formula I, II, III, or IV or a salt thereof. In certain embodiments, the compound is a compound of Formula I. In certain embodiments, the compound is a compound of Formula II. In certain embodiments, the compound is a compound of Formula III. In certain embodiments, the compound is a compound of Formula IV.

In certain embodiments, the pharmaceutical compositions preferably comprise a therapeutically-effective amount of one or more of the fatty acid cysteine-based conjugates described above, formulated together with one or more pharmaceutically acceptable carriers. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets (e.g., those targeted for buccal, sublingual, and/or systemic absorption), boluses, powders, granules, pastes for application to the tongue; (2) parenteral administration by, for example, subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration.

The tablets, and other solid dosage forms of the pharmaceutical compositions of the present invention, such as capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Administration of the conjugates of the invention can be accomplished via any suitable mode of administration for therapeutic agents. These modes include systemic or local administration such as oral, nasal, parenteral, transdermal, subcutaneous, vaginal, buccal, rectal or topical administration modes.

Depending on the intended mode of administration, the compositions can be in solid, semi-solid or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices. Likewise, they can also be administered in intravenous (both bolus and infusion), intraperitoneal, subcutaneous or intramuscular form, all using forms well known to those skilled in the pharmaceutical arts.

Illustrative pharmaceutical compositions are tablets and gelatin capsules comprising a fatty acid cysteine conjugate and a pharmaceutically acceptable carrier, such as: a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, alginic acid or its sodium salt, or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the compound such as cyclodextrin, hydroxypropyl-cyclodextrin, PEG400, PEG200.

Liquid, particularly injectable, compositions can, for example, be prepared by dissolution, dispersion, etc. For example, the fatty acid cysteine conjugate is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the fatty acid cysteamine conjugates.

The fatty acid cysteine-based conjugates can be also formulated as a suppository that can be prepared from fatty emulsions or suspensions; using polyalkylene glycols such as propylene glycol, as the carrier.

The fatty acid cysteine-based conjugates can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564.

Parenteral injectable administration is generally used for subcutaneous, intramuscular or intravenous injections and infusions. Injectables can be prepared in conventional forms, either as liquid solutions or suspensions or solid forms suitable for dissolving in liquid prior to injection.

Compositions can be prepared according to conventional mixing, granulating or coating methods, respectively, and the present pharmaceutical compositions can contain from about 0.1% to about 80%, from about 5% to about 60%, or from about 1% to about 20% of the conjugate by weight or volume.

The dosage regimen utilizing the fatty acid cysteine-based conjugates is selected in accordance with a variety of factors including type, species, age, weight, sex and medical condition of the patient; the severity of the condition to be treated; the route of administration; the renal or hepatic function of the patient; and the particular conjugate of CFTR modulator employed. A physician or veterinarian of ordinary skill in the art can readily determine and prescribe the effective amount of the drug required to prevent, counter or arrest the progress of the condition.

Effective dosage amounts of the present invention, when used for the indicated effects, range from about 20 mg to about 5,000 mg of the conjugate per day. Compositions for in vivo or in vitro use can contain about 20, 50, 75, 100, 150, 250, 500, 750, 1,000, 1,250, 2,500, 3,500, or 5,000 mg of the conjugate of a CFTR modulator. In one embodiment, the compositions are in the form of a tablet that can be scored. Effective plasma levels of the conjugates can range from about 5 ng/mL to 5000 ng/mL per day. Appropriate dosages of the conjugate can be determined as set forth in Goodman, L. S.; Gilman, A. (1975) THE PHARMACOLOGICAL BASIS OF THERAPEUTICS, 5th ed.; MacMillan: New York, pp. 201-226.

The conjugates of the invention can be administered in a single daily dose, or the total daily dosage can be administered in divided doses of two, three or four times daily. Furthermore, fatty acid cysteine-based conjugates can be administered in intranasal form via topical use of suitable intranasal vehicles, or via transdermal routes, using those forms of transdermal skin patches well known to those of ordinary skill in that art. To be administered in the form of a transdermal delivery system, the dosage administration can be continuous rather than intermittent throughout the dosage regimen. Other illustrative topical preparations include creams, ointments, lotions, aerosol sprays and gels, wherein the concentration of the fatty acid cysteine conjugate ranges from about 0.1% to about 15%, w/w or w/v.

### Combination Therapies

The conjugates of the invention may also be administered with other therapeutic agents such as CFTR modulators, epithelial sodium channel (ENaC) inhibitors, anti-inflammatory agents, anti-fibrotic agents and antibacterial agents. In some embodiments, the other therapeutic agent is a CFTR modulator. Non-limiting examples of a CFTR modulator include Ivacaftor (VX-770), Lumacaftor (VX-809), VX-661, Orkambi (the combination of VX-770 and VX-809), the combination of VX-661 and VX-770, VX-152, VX-440, the combination of VX-152/VX-809 and VX-770, the combination of VX-440/VX-809 and VX-770, P-1037, Riociguat, N91115, QBW251, QR-010, GLPG1837, GLPG2222, GLP2665, genistein, baicalein, epigallocatechin gallate (EGCG), trimethylangelicin and Ataluren.

In some embodiments, the other therapeutic agent is an anti-inflammatory agent. Non-limiting examples of an anti-inflammatory agent include ibuprofen, prednisolone, dexamethasone, hydrocortisone, methylprednisolone, beclometasone, budesonide, fluticasone, mometasone, Seretide (fluticasone plus salmeterol), Symbicort (budesonide plus formoterol) and N91115.

In some embodiments, the other therapeutic agent is an anti-bacterial agent. Non-limiting examples of an anti-bacterial agent include azithromycin, tobramycin, aztreonam lysine, colistin, aminoglycosides, vancomycin, ciprofloxacin, levofloxacin and sulfonamides.

In some embodiments, the other therapeutic agent is an epithelial sodium channel (ENaC) inhibitor. Non-limiting examples of ENaC inhibitors include amiloride, BA-39-9437, GS-9411 and P-1037.

In some embodiments, the other therapeutic agent is an anti-fibrotic agent. Non-limiting examples of anti-fibrotic agents include pirfenidone, nintedanib, INT-767, STX-100, AM152, pentoxyphilline, FG-3019, CNTO 888, Tralokinumab, SAR156597, GS-6624, BMS-986020, Lebrikizumab, GSK2126458, ACT-064992, vismodegib, PRM-151, IW001 and Fresolimumab.

### V. Kits for Use in Medical Applications

Another aspect of the invention provides a kit for treating a disorder. The kit comprises: i) instructions for treating a medical disorder, such as CF; and ii) a conjugate described herein. The kit may comprise one or more unit dosage forms containing an amount of a fatty acid cysteine conjugate described herein.

The description above describes multiple aspects and embodiments of the invention, including a fatty acid cysteine conjugate, compositions comprising a fatty acid cysteine conjugate, methods of using the fatty acid cysteine conjugate, and kits. The invention specifically contemplates all combinations and permutations of the aspects and embodiments.

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby.

### Example 1

### Preparation of N-(2-(((R)-3-amino-2-((4Z,7Z,10Z,13Z,16Z,19Z)-docosa-4,7,10,13,16,19-hexaenamido)-3-oxopropyl)disulfanyl)-2-methylpropyl)nicotinamide (I-1)

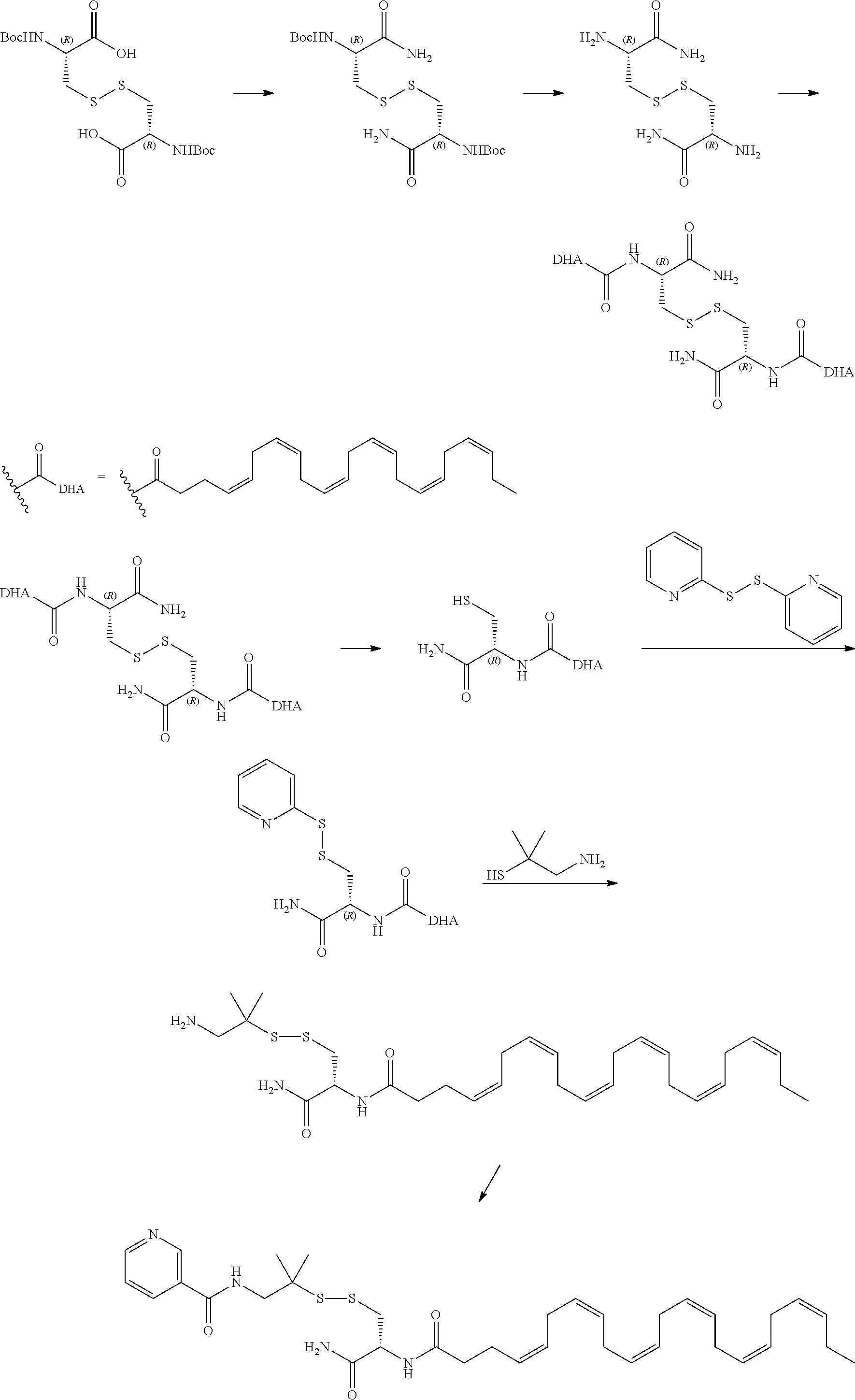

In a typical run, N,N′-Di-Boc-L-cystine (5.0 g, 11 mmol) was suspended in DMF (40 mL) and ammonium chloride (590 mg, 11 mmol) was added, followed by HATU (5.0 g, 13 mmol). DIEA was then added to adjust the pH to about 8. The resulting reaction mixture was stirred at room temperature for 16 hours. It was then diluted with H₂O (400 mL) and the precipitated solids were collected after filtration. The solids were washed with EtOAc (100 mL×2) and dried under reduced pressure to afford tert-butyl-(2R,2′R)-3,3′-disulfanediylbis(1-amino-1-oxopropane-3,2-diyl)dicarbamate (3.3 g, 70% yield). This disulfide intermediate (2.0 g, 4.6 mmol) was then taken up in CH₂Cl₂ (10 mL) and TFA (4 mL) was added. The resulting reaction mixture was stirred at room temperature for 4 hours and then concentrated under reduced pressure to afford (2R,2′R)-3,3′-disulfanediylbis(2-aminopropanamide) (2.2 g, ˜100% yield). This material was used for the next step without further purification. This material (2.2 g, 4.6 mmol) was taken up in DMF (20 mL) DHA (0.748 g, 2.3 mmol) was added, followed by HATU (1.5 g, 5.5 mmol). DIEA was added and the resulting reaction mixture was stirred at room temperature for 16 hours. The following day, the reaction mixture was diluted with H₂O (200 mL) and the resulting solids were collected by filtration. The solids were then washed with EtOAc and dried under reduced pressure to afford (4Z,4′Z,7Z,7′Z,10Z,10′Z,13Z,13′Z,16Z,16′Z,19Z,19′Z)—N,N′-((2R,2′R)-disulfanediylbis(3-amino-3-oxopropane-1,2-diyl))bis(docosa-4,7,10,13,16,19-hexaenamide) (2.0 g, 51% yield) as a yellow solid. This material (6.4 g, 21.6 mmol) was taken up in methanol (30 mL) and DTT (1.38 g, 9.00 mmol) was added under nitrogen at 0° C. Enough aqueous NaOH (1N) was added to the reaction mixture to maintain the pH to about 9. The resulting reaction mixture was allowed to warm to room temperature and stirred for 3 hours. The solvent was removed under reduced pressure to afford (4Z,7Z,10Z,13Z,16Z,19Z)—N—((R)-1-amino-3-mercapto-1-oxopropan-2-yl)docosa-4,7,10,13,16,19-hexaenamide (6.5 g, ˜100% yield).

This material was then used in the next step without further purification.

In a typical run, (4Z,7Z,10Z,13Z,16Z,19Z)—N—((R)-1-Amino-3-mercapto-1-oxopropan-2-yl)docosa-4,7,10,13,16,19-hexaenamide (3.2 g, 22 moml) was taken up in methanol (30 mL) and 1,2-di(pyridin-2-yl)disulfane (5.74 g, 26.1 mmol) was added at room temperature. The resulting reaction mixture was stirred at room temperature for 4 hours and then concentrated under reduced pressure. The resulting residue was diluted with H₂O and extracted twice with EtOAc. The combined organic layers were washed with brine, dried over anhydrous Na₂SO₄ and concentrated under reduced pressure. The resulting crude product was purified by silica gel chromatography using a mixture of CH₂Cl₂/MeOH to afford (4Z,7Z,10Z,13Z,16Z,19Z)—N—((R)-1-amino-1-oxo-3-(pyridin-2-yldisulfanyl)propan-2-yl)docosa-4,7,10,13,16,19-hexaenamide (3.8 g, 70% yield). This material (3.8 g, 14.6 mmol) was taken up in methanol (25 mL) and 1-amino-2-methylpropane-2-thiol (2.1 g, 14.6 mmol) was added. The resulting reaction mixture was stirred at room temperature for 2 hours and then concentrated under reduced pressure to afford (4Z,7Z,10Z,13Z,16Z,19Z)—N—((R)-1-amino-3-((1-amino-2-methylpropan-2-yl)disulfanyl)-1-oxopropan-2-yl)docosa-4,7,10,13,16,19-hexaenamide (3.7 g, ˜100% yield). This material was then used for the next step without further purification. In order to obtain the titled compound, (4Z,7Z,10Z,13Z,16Z,19Z)—N—((R)-1-amino-3-((1-amino-2-methylpropan-2-yl)disulfanyl)-1-oxopropan-2-yl)docosa-4,7,10,13,16,19-hexaenamide (609 mg, 2.4 mmol) was taken up in CH₂Cl₂ (40 mL) and nicotinic acid (295 mg, 2.4 mmol) was added, followed by HATU (1.1 g, 2.9 mmol) at 0° C. The reaction mixture was allowed to warm to room temperature and stirred for 16 hours. The solvent was removed by concentration under reduced pressure. The resulting residue was diluted with H₂O (100 mL) and extracted twice with EtOAc. The combined organic layers were washed with brine, dried over anhydrous Na₂SO₄ and concentrated under reduced pressure. The resulting residue was purified by preparative HPLC to afford N-(2-(((R)-3-amino-2-((4Z,7Z,10Z,13Z,16Z,19Z)-docosa-4,7,10,13,16,19-hexaenamido)-3-oxopropyl)disulfanyl)-2-methylpropyl)nicotinamide (800 mg, 52.4% yield) as yellow oil. MS, calculated for C₃₅H₅₀N₄O₃S₂: 638.33; found 639 [M+H]⁺.

### Example 2

### Preparation of 4-((2-(((R)-3-amino-2-((4Z,7Z,10Z,13Z,16Z,19Z)-docosa-4,7,10,13,16,19-hexaenamido)-3-oxopropyl)disulfanyl)-2-methylpropyl)amino)-2-hydroxy-N,N,N-trimethyl-4-oxobutan-1-aminium (I-5)

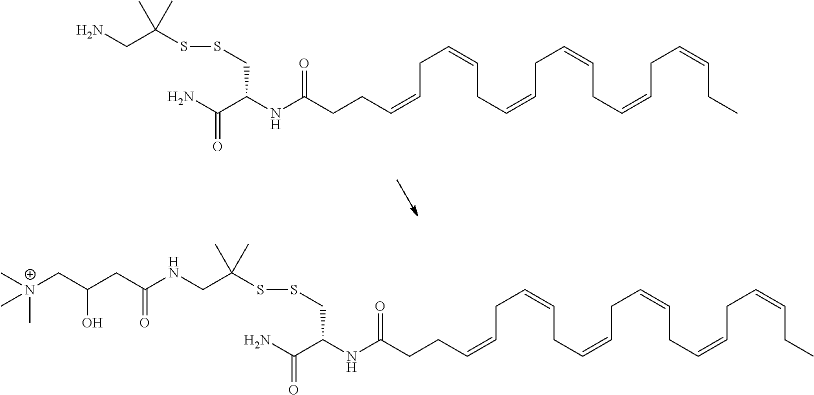

The same experimental procedure outlined in example 1 was used to prepare the key intermediate (4Z,7Z,10Z,13Z,16Z,19Z)—N—((R)-1-amino-3-((1-amino-2-methylpropan-2-yl)disulfanyl)-1-oxopropan-2-yl)docosa-4,7,10,13,16,19-hexaenamide. For the final amide coupling step, L-carnitine was used instead of nicotinic acid. MS, calculated for C₃₆H₆₁N₄O₄S₂: 677.41; found 678 [M+H]⁺.

### Example 3

### Preparation of N-((4Z,7Z,10Z,13Z,16Z,19Z)-docosa-4,7,10,13,16,19-hexaenoyl)-S-((2-methyl-1-(nicotinamido)propan-2-yl)thio)-L-cysteine (I-3)

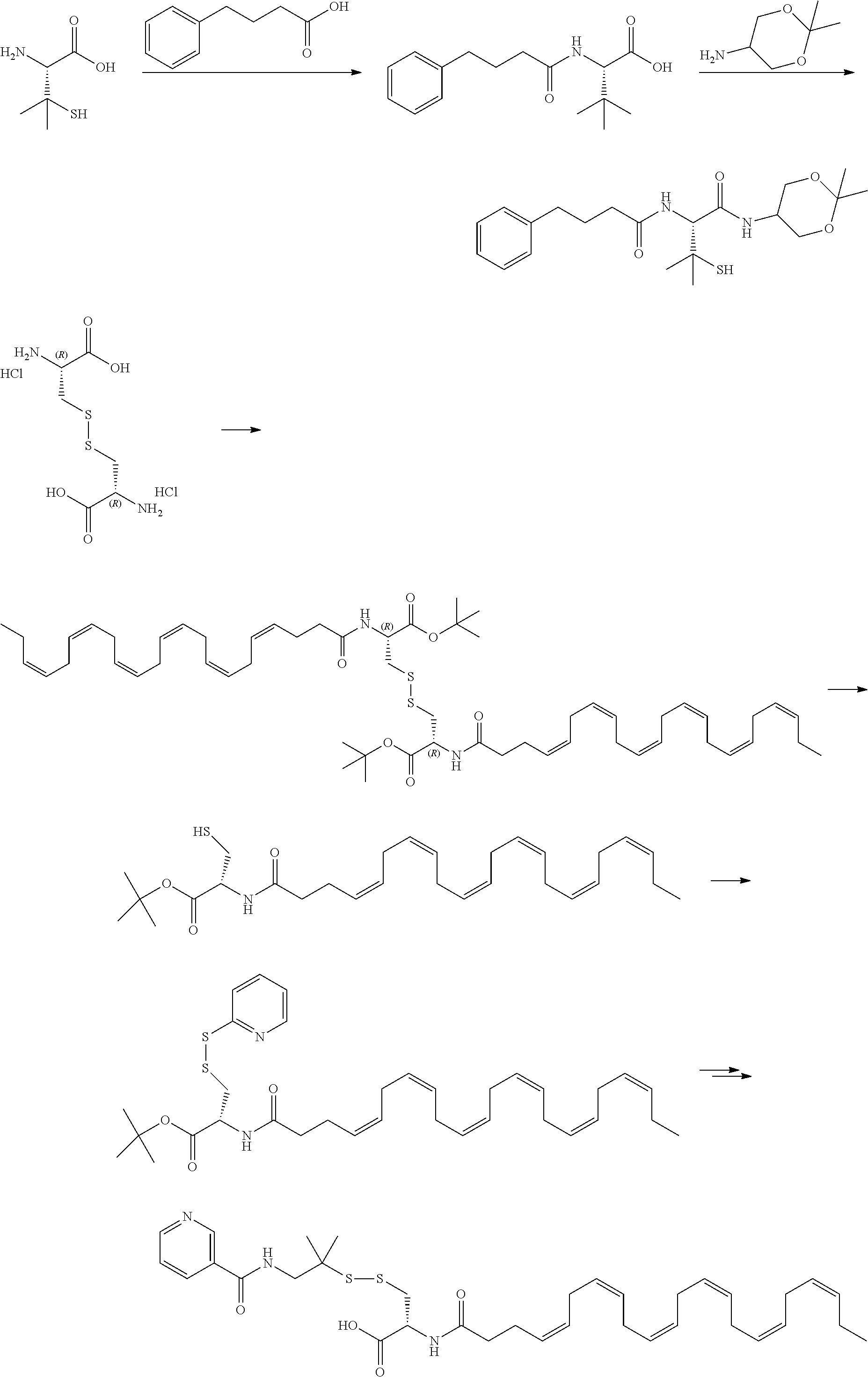

In a typical run, (R)-2-amino-3-mercapto-3-methylbutanoic acid (0.454 g, 3.05 mmol, 1 eq) and 4-phenylbutyric acid (0.5 g, 3.05 mmol, 1 eq) were taken up in DMF (10 mL). HATU (1.51 g, 3.96 mmol, 1.3 eq) and Et₃N (0.924 g, 9.15 mmol, 3 eq) were then added. The resulting reaction mixture was stirred at room temperature for 16 h under an inert atmosphere of nitrogen. The reaction mixture was extracted with EtOAc. The combined organic layers were washed with water and brine, dried over anhydrous Na₂SO₄ and concentrated under reduced pressure. The resulting residue was purified by preparative HPLC to afford (R)-3-mercapto-3-methyl-2-(4-phenylbutanamido)butanoic acid (0.35 g, 39% yield) as a yellow oil. This material (0.35 g, 1.19 mmol, 1 eq) was taken up in DMF (5 mL) and 2,2-dimethyl-1,3-dioxan-5-amine (0.17 g, 1.3 mmol, 1.1 eq) was added, followed by HATU (0.586 g, 1.54 mmol, 1.3 eq) and Et₃N (0.36 g, 3.56 mmol, 3 eq). The resulting reaction mixture was stirred at room temperature for 16 hours. The following day, the reaction mixture was extracted with EOAc. The combined organic layers were washed with water, brine, dried over anhydrous Na₂SO₄ and concentrated under reduced pressure. The resulting residue was purified by preparative HPLC to afford (R)—N-(2,2-dimethyl-1,3-dioxan-5-yl)-3-mercapto-3-methyl-2-(4-phenylbutanamido)butanamide (0.091 g, 18.8% yield) as a white solid.

The commercially available di-tert-butyl 3,3′-disulfanediyl(2R,2′R)-bis(2-aminopropanoate) (30 g, 70.58 mmol, 1 eq) was taken up in CH₂Cl₂ (600 mL) and DHA (53.3 g, 162.33 mmol, 2.3 eq) was added, followed by HATU (80 g, 211.74 mmol, 3 eq) and Et₃N (49 mL, 352.9 mmol, 5 eq). The resulting reaction mixture was stirred at room temperature for 16 hours. The following day, the reaction mixture was extracted with EtOAc. The combined organic layers were washed with water, brine, dried over anhydrous Na₂SO₄ and concentrated under reduced pressure. The resulting residue was purified by silica gel chromatography (CH₂Cl₂/MeOH) to afford di-tert-butyl 3,3′-disulfanediyl(2R,2′R)-bis(2-((4Z,7Z,10Z,13Z,16Z,19Z)-docosa-4,7,10,13,16,19-hexaenamido)propanoate) (61 g, 88.9% yield) as a yellow oil.

Di-tert-butyl 3,3′-disulfanediyl(2R,2′R)-bis(2-((4Z,7Z,10Z,13Z,16Z,19Z)-docosa-4,7,10,13,16,19-hexaenamido)propanoate) (20.66 g, 21.3 mmol, 1 eq) was taken up in EtOH (300 mL) and DTT (3.6 g, 23.4 mmol, 1.1 eq) was added. Enough 1N NaOH was added to adjust the pH to about 8.5-9. The resulting reaction mixture was stirred at room temperature for 20 min and then concentrated under reduced pressure to afford tert-butyl ((4Z,7Z,10Z,13Z,16Z,19Z)-docosa-4,7,10,13,16,19-hexaenoyl)-L-cysteinate in quantitative yield. This material was used in the next step without further purification. tert-Butyl ((4Z,7Z,10Z,13Z,16Z,19Z)-docosa-4,7,10,13,16,19-hexaenoyl)-L-cysteinate (20.66 g, 42.4 mmol, 1 eq) was taken up in MeOH (200 mL) and 1,2-di(pyridin-2-yl)disulfane (5.14 g, 23.3 mmol, 0.55 eq) was added. The resulting reaction mixture was stirred at room temperature for 6 h and then concentrated under reduced pressure. The resulting residue was purified by silica gel chromatography (pentanes/EtOAc) to afford tert-butyl N-((4Z,7Z,10Z,13Z,16Z,19Z)-docosa-4,7,10,13,16,19-hexaenoyl)-S-(pyridin-2-ylthio)-L-cysteinate (9.4 g, 37% yield) as a yellow oil. This material (0.091 g, 0.223 mmol, 1 eq) and (R)—N-(2,2-dimethyl-1,3-dioxan-5-yl)-3-mercapto-3-methyl-2-(4-phenylbutanamido)butanamide (0.133 g, 0.223 mmol, 1 eq) were taken up in DMF (2 mL) and methanol (2 mL). The resulting reaction mixture was stirred at room temperature for 4 h and then concentrated under reduced pressure. The resulting residue was purified by preparative HPLC to afford tert-butyl S—(((R)-4-((2,2-dimethyl-1,3-dioxan-5-yl)amino)-2-methyl-4-oxo-3-(4-phenylbutanamido)butan-2-yl)thio)-N-((4Z,7Z,10Z,13Z,16Z,19Z)-docosa-4,7,10,13,16,19-hexaenoyl)-L-cysteinate (0.15 g, 75.4% yield) as a colorless oil. This material (0.15 g, 0.168 mmol, 1 eq) was taken up in CH₂Cl₂ (5 mL) and TFA (1 mL) was added. The resulting reaction mixture was stirred at room temperature for 4 hours and then concentrated under reduced pressure. The resulting residue was purified by preparative HPLC to afford N-((4Z,7Z,10Z,13Z,16Z,19Z)-docosa-4,7,10,13,16,19-hexaenoyl)-S-((2-methyl-1-(nicotinamido)propan-2-yl)thio)-L-cysteine. MS, calculated for C₃₅H₄₉N₃O₄S₂: 639.32; found 640 [M+H]⁺.

### Example 4

### Preparation of 4-((3-(((R)-3-amino-2-((4Z,7Z,10Z,13Z,16Z,19Z)-docosa-4,7,10,13,16,19-hexaenamido)-3-oxopropyl)disulfanyl)-1-((1,3-dihydroxypropan-2-yl)amino)-3-methyl-1-oxobutan-2-yl)amino)-2-hydroxy-N,N,N-trimethyl-4-oxobutan-1-aminium (I-6)

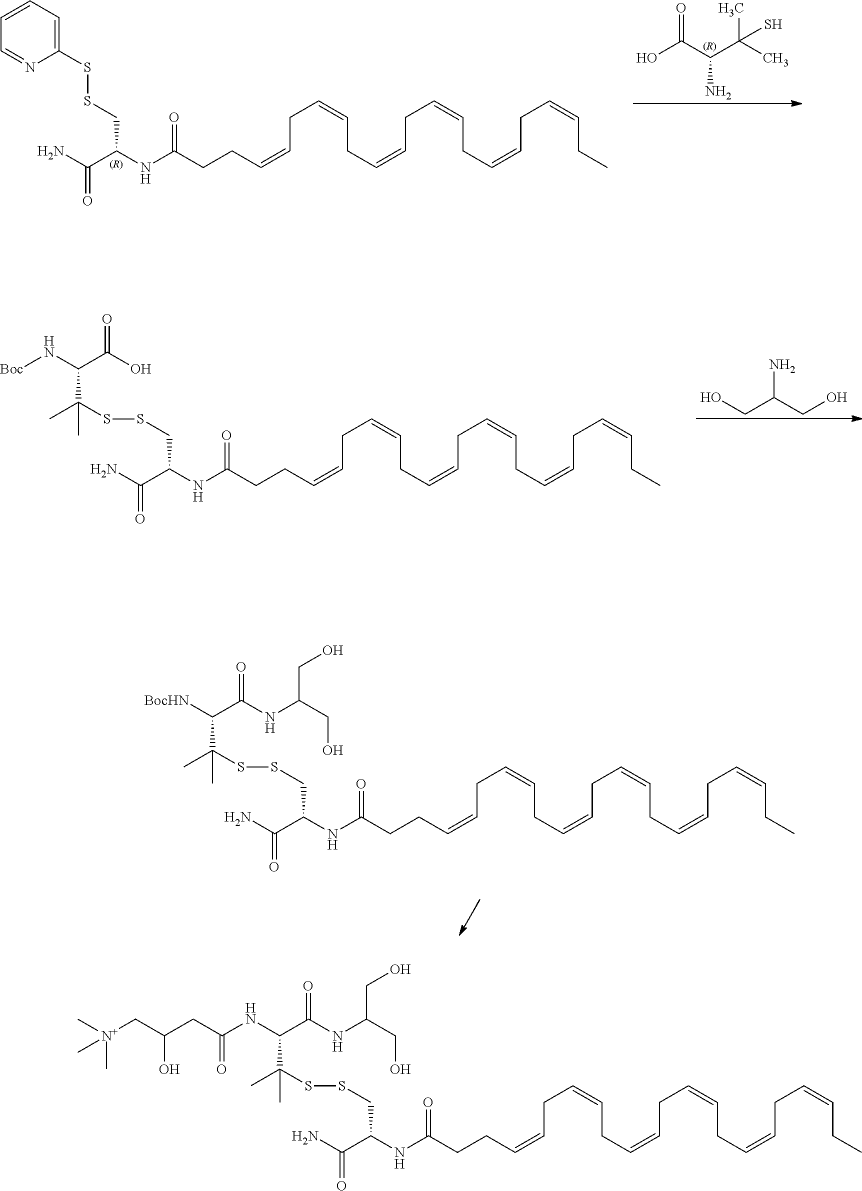

(4Z,7Z,10Z,13Z,16Z,19Z)—N—((R)-1-amino-1-oxo-3-(pyridin-2-yldisulfanyl)propan-2-yl)docosa-4,7,10,13,16,19-hexaenamide was prepared according to the procedures outlined in Example 1. This intermediate (5.0 g, 9.3 mmol) was taken up in methanol (50 mL) and (R)-2-amino-3-mercapto-3-methylbutanoic acid (1.38 g, 9.3 mmol) was added at room temperature. The resulting reaction mixture was stirred at room temperature for 3 hours and then concentrated under reduced pressure to afford (R)-2-amino-3-(((R)-3-amino-2-((4Z,7Z,10Z,13Z,16Z,19Z)-docosa-4,7,10,13,16,19-hexaenamido)-3-oxopropyl)disulfanyl)-3-methylbutanoic acid (9.3 mol). This material was used in the next step without further purification.

A suspension of (R)-2-amino-3-(((R)-3-amino-2-((4Z,7Z,10Z,13Z,16Z,19Z)-docosa-4,7,10,13,16,19-hexaenamido)-3-oxopropyl)disulfanyl)-3-methylbutanoic acid (9.3 mmol) in methanol (30 mL) was cooled in an ice bath and 1N aqueous NaOH (25 mmol) was added. This was followed by the addition of di-tert-butyl dicarbonate (3.12 g, 14 mmol). The resulting reaction mixture was allowed to warm to room temperature and stirred for 3 hours. The mixture was then acidified with HCl (aq) to a pH of about 5. The resulting mixture was extracted with EtOAc (3×100 mL). The combined organic layers were washed with and brine, dried over anhydrous Na₂SO₄ and concentrated under reduced pressure. The resulting residue was purified by silica gel chromatography (gradient elution, methanol: CH₂Cl₂₌=1.5%˜2.2%) to afford (R)-3—(((R)-3-amino-2-((4Z,7Z,10Z,13Z,16Z,19Z)-docosa-4,7,10,13,16,19-hexaenamido)-3-oxopropyl)disulfanyl)-2-((tert-butoxycarbonyl)amino)-3-methylbutanoic acid (4.5 g, 53% yield). This material (3.8 g, 5.6 mmol) was taken up in CH₂Cl₂ (30 mL) and 2-aminopropane-1,3-diol (0.51 g, 5.6 mmol) was added at room temperature, followed by HATU (2.6 g, 6.7 mmol) and DIEA (7.5 mmol). The resulting reaction mixture was stirred at room temperature for 16 hours. The following day, the mixture was diluted with CH₂Cl₂, washed with H₂O and brine, dried over anhydrous Na₂SO₄ and concentrated under reduced pressure to afford tert-butyl ((R)-3-(((R)-3-amino-2-((4Z,7Z,10Z,13Z,16Z,19Z)-docosa-4,7,10,13,16,19-hexaenamido)-3-oxopropyl)disulfanyl)-1-((1,3-dihydroxypropan-2-yl)amino)-3-methyl-1-oxobutan-2-yl)carbamate (5.6 mmol, ˜100% yield). This material was used for the next step without further purification. tert-butyl ((R)-3-(((R)-3-Amino-2-((4Z,7Z,10Z,13Z,16Z,19Z)-docosa-4,7,10,13,16,19-hexaenamido)-3-oxopropyl)disulfanyl)-1-((1,3-dihydroxypropan-2-yl)amino)-3-methyl-1-oxobutan-2-yl)carbamate (5.6 mmol) was treated with 4 N HCl-dioxane (5 mL) at room temperature for 1 h and then concentrated under reduced pressure to afford the BOC-deprotected intermediate. This material (4.2 g, 5.6 mmol) taken up in CH₂Cl₂ (20 mL) and L-carnitine (0.91 g, 5.6 mmol) was added at room temperature, followed by HATU (2.6 g, 6.7 mmol) and DIEA (8 mmol). The resulting reaction mixture was stirred at room temperature for 16 hours. The following day, the reaction mixture was concentrated under reduced pressure and the resulting crude product was purified by preparative HPLC to afford 4-((3-(((R)-3-amino-2-((4Z,7Z,10Z,13Z,16Z,19Z)-docosa-4,7,10,13,16,19-hexaenamido)-3-oxopropyl)disulfanyl)-1-((1,3-dihydroxypropan-2-yl)amino)-3-methyl-1-oxobutan-2-yl)amino)-2-hydroxy-N,N,N-trimethyl-4-oxobutan-1-aminium (850 mg, 19% yield) as a white solid. MS, calculated for C₄₀H₆₈N₅O₇S₂: 794.46; found 795 [M+H]⁺.

### Example 5

### Preparation of (4Z,7Z,10Z,13Z,16Z,19Z)—N—((R)-1-amino-3-(((R)-4-(((R)-2,3-dihydroxypropyl)amino)-2-methyl-4-oxo-3-(4-phenylbutanamido)butan-2-yl)disulfanyl)-1-oxopropan-2-yl)docosa-4,7,10,13,16,19-hexaenamide (I-10)

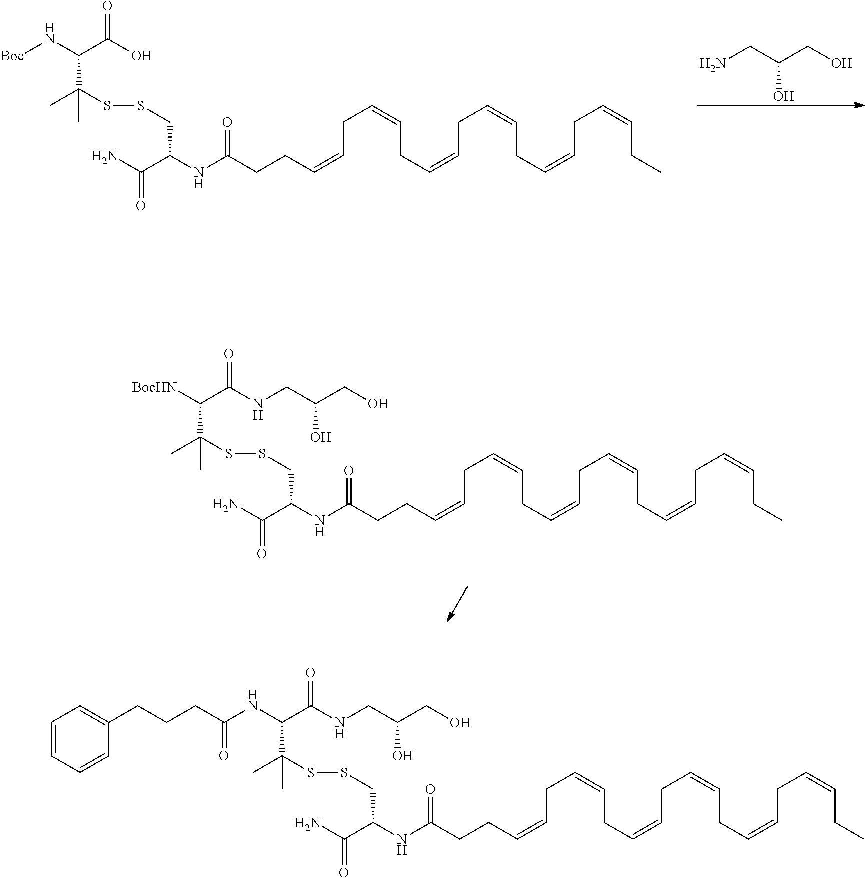

The same experimental procedure outlined in example 4 was used, substituting 2-aminopropane-1,3-diol and L-carnitine with (R)-3-aminopropane-1,2-diol and 4-phenylbutyric acid respectively. The final product was purified by silica gel chromatography (CH₂Cl₂/MeOH). MS, calculated for C₄₃H₆₄N₄O₆S₂: 796.43; found 797 [M+H]⁺.

### Example 6

### Preparation of N—((R)-3-(((R)-3-amino-2-((4Z,7Z,10Z,13Z,16Z,19Z)-docosa-4,7,10,13,16,19-hexaenamido)-3-oxopropyl)disulfanyl)-1-(((R)-2,3-dihydroxypropyl)amino)-3-methyl-1-oxobutan-2-yl)-1-(2,2-difluorobenzo[d][1,3]dioxol-5-yl)cyclopropane-1-carboxamide (I-12)

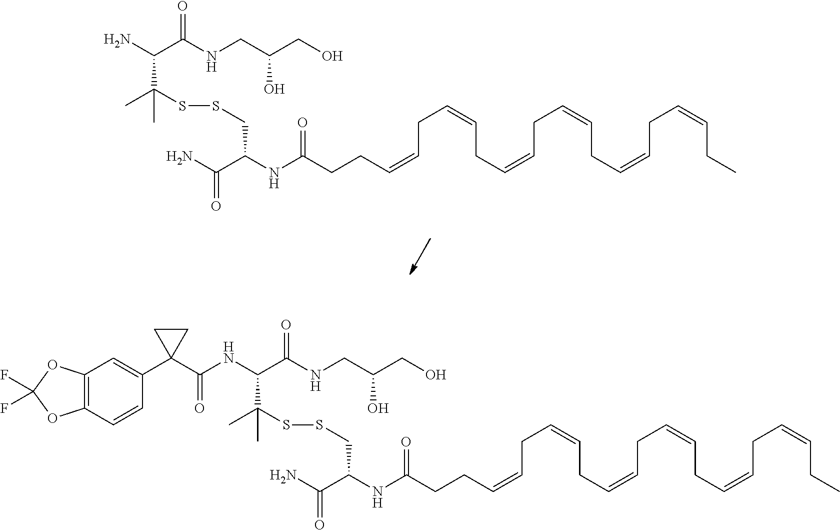 [SMILES: CC/C=C\C/C=C\C/C=C\C/C=C\C/C=C\C/C=C\CCC(=O)N[C@@H](CSSC(C)(C)[C@H](N)C(=O)NC[C@@H](O)CO)C(N)=O ; CC/C=C\C/C=C\C/C=C\C/C=C\C/C=C\C/C=C\CCC(=O)N[C@@H](CSSC(C)(C)[C@H](NC(=O)C1(c2ccc3c(c2)OC(F)(F)O3)CC1)C(=O)NC[C@@H](O)CO)C(N)=O]

(4Z,7Z,10Z,13Z,16Z,19Z)—N—((R)-1-Amino-3-(((R)-3-amino-4-(((R)-2,3-dihydroxypropyl)amino)-2-methyl-4-oxobutan-2-yl)disulfanyl)-1-oxopropan-2-yl)docosa-4,7,10,13,16,19-hexaenamide was prepared according to the procedures outlined in examples 4 and 5. This material was coupled with the commercially available 1-(2,2-difluorobenzo[d][1,3]dioxol-5-yl)cyclopropane-1-carboxylic acid using the same amide coupling procedure outlined in example 4. The titled compound was obtained after silica gel chromatography (CH₂Cl₂/MeOH). MS, calculated for C₄₄H₆₀F₂N₄O₈S₂: 874.38; found 875 [M+H]⁺.

### Example 7

### Preparation of N—((R)-3-(((R)-3-amino-2-((4Z,7Z,10Z,13Z,16Z,19Z)-docosa-4,7,10,13,16,19-hexaenamido)-3-oxopropyl)disulfanyl)-1-((1,3-dihydroxypropan-2-yl)amino)-3-methyl-1-oxobutan-2-yl)-5-phenylisoxazole-3-carboxamide (I-15)

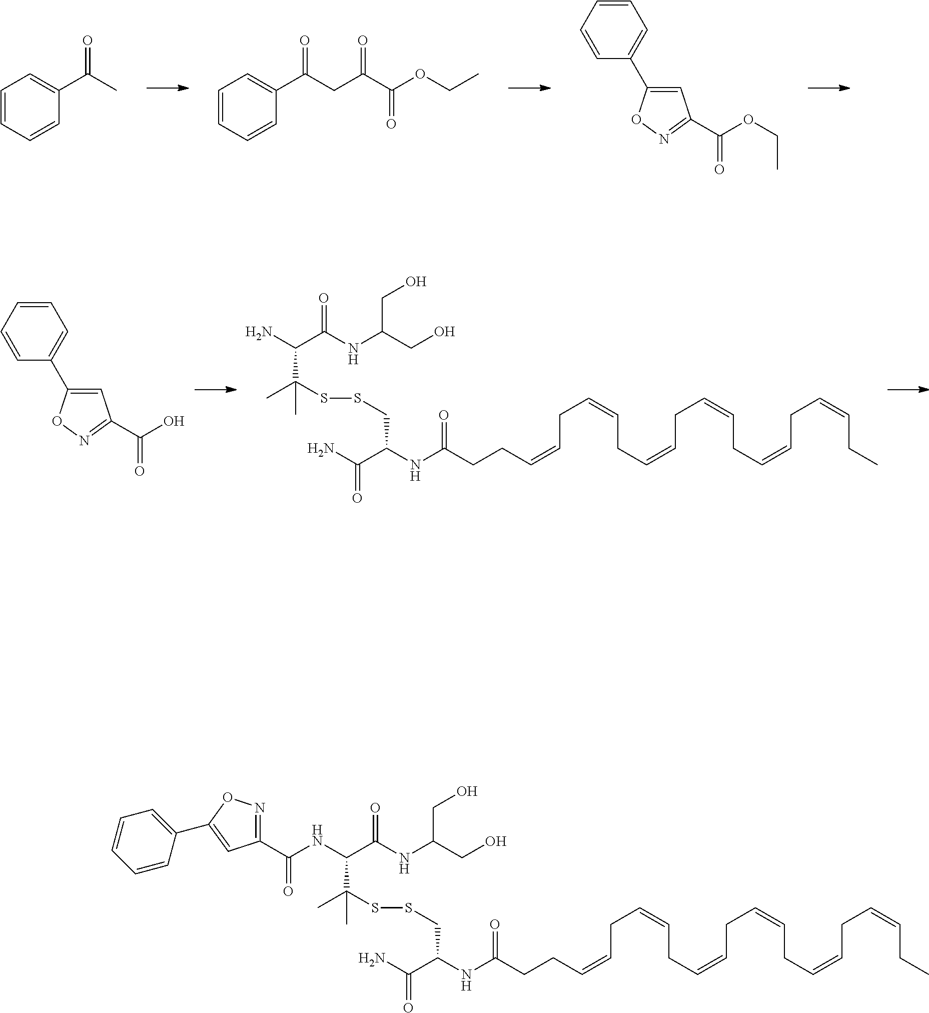

Acetophenone (3 g, 25 mmol) was taken up in 30 mL of dry toluene and NaH (780 mg, 32 mmol) was then added. The resulting reaction mixture was stirred at room temperature for 60 minutes. A solution of diethyl oxalate (5.5 g, 37.5 mmol) in dry toluene (25 mL) was then added drop wise and stirred at room temperature for 1 hour. The reaction mixture was concentrated under reduced pressure and the resulting residue was diluted with ice water. The precipitated solids were collected by filtration and dried to afford 2.85 g of ethyl 2,4-dioxo-4-phenylbutanoate (52% yield) as a yellow solid. This material (2.85 g, 12.9 mmol) was taken up in EtOH (25 mL) along with NH₂OH.HCl (1.16 g, 16.8 mmol) and then stirred under reflux for 3 hours. The reaction mixture was concentrated under reduced pressure. The resulting residue was diluted with water and extracted with EtOAc. The combined organic layers were washed with H₂O, dried (Na₂SO₄) and concentrated under reduced pressure. Purification by silica gel chromatography (pentanes/EtOAc) afforded ethyl 5-phenylisoxazole-3-carboxylate (2.53 g, 90% yield) as a white solid. This material (2.53 g, 11.6 mmol) was taken up in THF/H₂O (45 mL/5 mL) along with LiOH.H₂O (1.0 g, 23.3 mmol) and the resulting reaction mixture was stirred at room temperature for 2 hours. The reaction mixture was then concentrated under reduced pressure. Sufficient 1 N HCl was added to the resulting residue to bring the pH to about 5. The resulting solids were collected by filtration and dried under high vacuum to afford 1.5 g of 5-phenylisoxazole-3-carboxylic acid (69%) as a white solid. 5-Phenylisoxazole-3-carboxylic acid was then coupled with (4Z,7Z,10Z,13Z,16Z,19Z)—N—((R)-1-amino-3-(((R)-3-amino-4-((1,3-dihydroxypropan-2-yl)amino)-2-methyl-4-oxobutan-2-yl)disulfanyl)-1-oxopropan-2-yl)docosa-4,7,10,13,16,19-hexaenamide using the same amide coupling procedure as detailed in example 4. The final product was purified by silica gel chromatography (CH₂Cl₂/MeOH). MS, calculated for C₄₃H₅₉N₅O₇S₂: 821.39; found 822 [M+H]⁺.

### Example 8

### Preparation of S—(((R)-3-(1-(2,2-difluorobenzo[d][1,3]dioxol-5-yl)cyclopropane-1-carboxamido)-4-(((R)-2,3-dihydroxypropyl)amino)-2-methyl-4-oxobutan-2-yl)thio)-N-((4Z,7Z,10Z,13Z,16Z,19Z)-docosa-4,7,10,13,16,19-hexaenoyl)-L-cysteine (I-14)

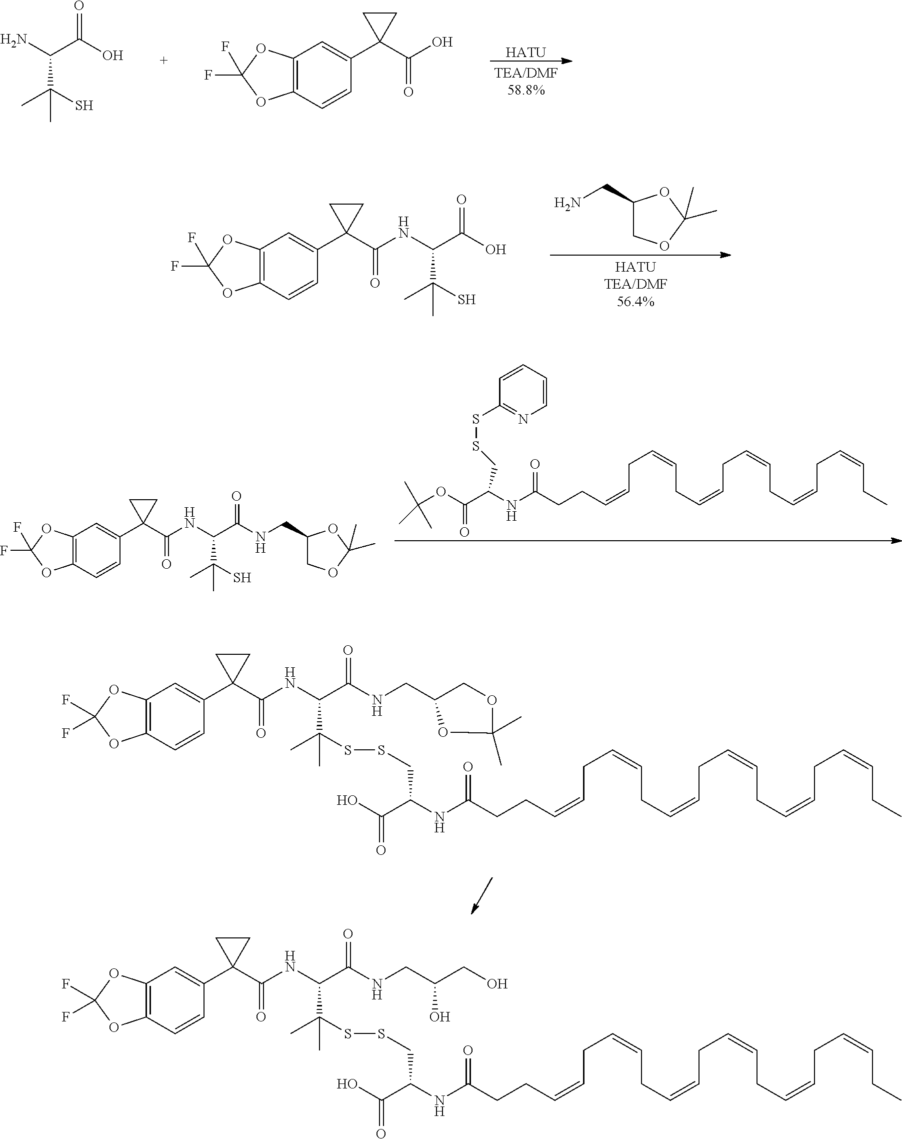

In a typical run, (R)-2-amino-3-mercapto-3-methylbutanoic acid (0.25 g, 1.68 mmol, 1 eq) and 1-(2,2-difluorobenzo[d][1,3]dioxol-5-yl)cyclopropane-1-carboxylic acid (0.45 g, 1.85 mmol, 1.1 eq) were taken up in DMF (10 mL) and HATU (0.83 g, 2.18 mmol, 1.3 eq) was added, followed by Et₃N (0.508 g, 5.03 mmol, 3 eq). The resulting reaction mixture was stirred at room temperature for 16 hours. The following morning, the reaction mixture was extracted with EtOAc. The combined organic layers were washed with water, brine, dried over anhydrous Na₂SO₄ and concentrated under reduced pressure. The resulting residue was purified by preparative HPLC afford (R)-2-(1-(2,2-difluorobenzo[d][1,3]dioxol-5-yl)cyclopropane-1-carboxamido)-3-mercapto-3-methylbutanoic acid (0.368 g, 59% yield) as a white solid. This material (0.368 g, 0.987 mmol, 1 eq) and (R)-(2,2-dimethyl-1,3-dioxolan-4-yl)methanamine (0.155 g, 1.2 mmol, 1.1 eq) were taken up in DMF (5 mL) and HATU (0.487 g, 1.28 mmol, 1.3 eq) was added, followed by Et₃N (0.299 g, 2.96 mmol, 3 eq). The resulting reaction mixture was stirred at room temperature for 16 hours. The following morning, the reaction mixture was extracted with EtOAc. The combined organic layers were washed with water, brine, dried over anhydrous Na₂SO₄ and concentrated and concentrated under reduced pressure. The resulting residue was purified by preparative HPLC to afford 1-(2,2-difluorobenzo[d][1,3]dioxol-5-yl)-N—((R)-1-((((R)-2,2-dimethyl-1,3-dioxolan-4-yl)methyl)amino)-3-mercapto-3-methyl-1-oxobutan-2-yl)cyclopropane-1-carboxamide (0.27 g, 56% yield) as a colorless oil. This material (0.27 g, 0.556 mmol, 1 eq) was taken up in DMF (2 mL) along with tert-butyl N-((4Z,7Z,10Z,13Z,16Z,19Z)-docosa-4,7,10,13,16,19-hexaenoyl)-S-(pyridin-2-ylthio)-L-cysteinate (0.365 g, 0.611 mmol, 1.1 eq) and methanol (2 mL) was added. tert-butyl N-((4Z,7Z,10Z,13Z,16Z,19Z)-Docosa-4,7,10,13,16,19-hexaenoyl)-S-(pyridin-2-ylthio)-L-cysteinate, in turn, was prepared according to the procedures outlined in example 3. The resulting reaction mixture was stirred at room temperature for 16 hours and then concentrated under reduced pressure. The resulting residue was purified by preparative HPLC to afford tert-butyl S—(((R)-3-(1-(2,2-difluorobenzo[d][1,3]dioxol-5-yl)cyclopropane-1-carboxamido)-4-((((R)-2,2-dimethyl-1,3-dioxolan-4-yl)methyl)amino)-2-methyl-4-oxobutan-2-yl)thio)-N-((4Z,7Z,10Z,13Z,16Z,19Z)-docosa-4,7,10,13,16,19-hexaenoyl)-L-cysteinate (0.22 g, 40.8% yield) as a colorless oil. This material (0.22 g, 0.467 mmol, 1 eq) was taken up in CH₂Cl₂ (5 mL) and TFA (1 mL) was added. The resulting reaction mixture was stirred at room temperature for 4 hour and then concentrated under reduced pressure. The resulting residue was purified by preparative HPLC to afford S—(((R)-3-(1-(2,2-difluorobenzo[d][1,3]dioxol-5-yl)cyclopropane-1-carboxamido)-4-(((R)-2,3-dihydroxypropyl)amino)-2-methyl-4-oxobutan-2-yl)thio)-N-((4Z,7Z,10Z,13Z,16Z,19Z)-docosa-4,7,10,13,16,19-hexaenoyl)-L-cysteine (0.02 g, 10% yield) as a colorless oil. MS, calculated for C₄₄H₅₉F₂N₃O₉S₂: 875.37; found 876 [M+H]⁺.

### Example 9

### Preparation of S—(((R)-4-(((R)-2,3-dihydroxypropyl)amino)-2-methyl-4-oxo-3-(4-phenylbutanamido)butan-2-yl)thio)-N-((4Z,7Z,10Z,13Z,16Z,19Z)-docosa-4,7,10,13,16,19-hexaenoyl)-L-cysteine (I-20)

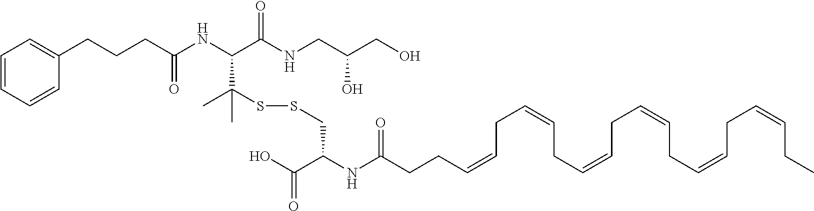 [SMILES: CC/C=C\C/C=C\C/C=C\C/C=C\C/C=C\C/C=C\CCC(=O)N[C@@H](CSSC(C)(C)[C@H](NC(=O)CCCc1ccccc1)C(=O)NC[C@@H](O)CO)C(=O)O]

The same experimental procedure outlined in example 8 was used, substituting 1-(2,2-difluorobenzo[d][1,3]dioxol-5-yl)cyclopropane-1-carboxylic acid with 4-phenylbutyric acid. MS, calculated for C₄₃H₆₃N₃O₇S₂: 797.41; found 798 [M+H]⁺.

### Example 10

### Metabolic Flux Analysis: Measurements for Improvement in Mitochondrial Bioenergetics

C2C12 cells were cultured in Sea Horse XF96 plates according to the manufacturer's instructions and established protocols (see www.seahorsebio.com/products/instruments/selection.php; Yao et al., PROC. NATL. ACAD. SCI. USA (2009) 106, p. 14670-14675).

On the day of the assay, cells were incubated with the compounds of the invention for either 4 hours or 24 hours. The short 4 hour incubation with the test compounds allowed one to evaluate for improvement of mitochondrial bioenergetics under the “scavengers” conditions (i.e. conditions in which the test compounds could effectively scavenge the various reactive oxygen species). The longer 24 hour incubation with the test compounds allowed one to evaluate for improvement of mitochondrial bioenergetics under the Nrf2 conditions (i.e. conditions in which the test compounds could effectively activate the Nrf2 antioxidant pathway in order to cope with the various reactive oxygen species). For these assays, H₂O₂ was used as the source of reactive oxygen species. Under the general stress test, cells were then exposed to either 500 μM of H₂O₂ or 1 mM of H₂O₂ for 4 hours before the evaluation in the Sea Horse system. Baseline measurements of the oxygen consumption rate (OCR) were taken before sequential injections of the various mitochondrial inhibitors. Readings were taken after addition of the mitochondrial inhibitor before injection of the subsequent inhibitors. The mitochondrial inhibitors that were used included oligomycin (1 μM), FCCP (1 μM) and Antimycin A (1 μM). Oligomycin is a known inhibitor ATP synthase; addition of this reagent essentially stopped the synthesis of ATP. Antimycin A inhibits the electron transport chain in the mitochondria. FCCP essentially uncouples the mitochondria and allows one to measure the maximum respiration. Measurements of the OCR following the addition of FCCP allows one to assess the mitochondrial content. An improvement in the OCR following the addition of FCCP is a reflection of the improvement in bioenergetics, when the proper control groups were put in place. The OCR were automatically calculated and recorded by the Sea Horse XF96 software. Ten measurements were taken at each of the indicated time points. Statistical significance in different treatment groups were determined by an ANOVA, followed by a Newman-Keuls post hoc analysis. Compounds I-1, I-3 and 1-5 were evaluated for mitochondrial bioenergetics under various conditions. The results for each are discussed below.

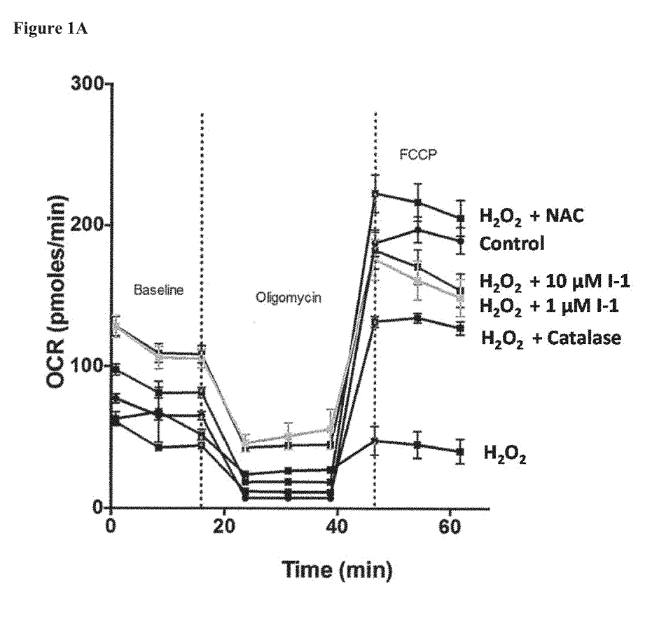

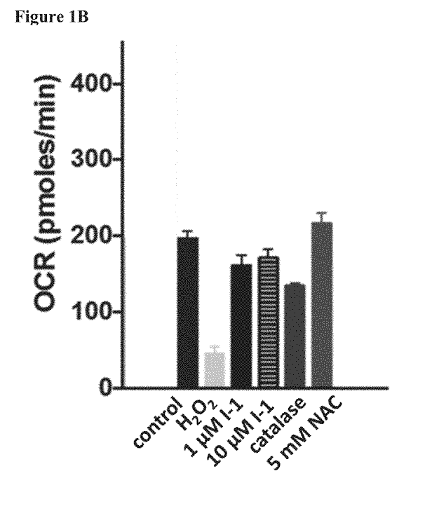

FIG. 1A shows the OCR data over time when C2C12 cells were treated for 4 hours with either 1) vehicle group; 2) 1 μM of compound I-1; 3) 10 μM of compound I-1; 4) positive control consisting of 5 mM of N-acetyl cysteine (NAC); 5) positive control consisting of 5K U/mL of the enzyme catalase. Each of these 5 treatment groups were exposed to a source of reactive oxygen species consisting of 500 μM of H₂O₂ as a stress. A 6^{th} treatment group, the “control group”, consisted of C2C12 cells that have been treated with the vehicle group but not exposed to 500 μM of H₂O₂. As shown in FIG. 1A, the control group shows how normal C2C12 cells would respond to the different mitochondrial inhibitors. Normal C2C12 has a baseline respiration, which was effectively inhibited by the addition of oligomycin. Addition of FCCP resulted in maximal respiration and a large increase in the OCR over time. When cells were treated with the vehicle and later stressed with H₂O₂ (treatment group 1), there was a much smaller increase in the OCR upon the addition of FCCP. When cells were treated with the positive control groups (catalase or NAC), there was a significant restoration of the OCR upon the addition of FCCP, almost to the same level as the normal “control group”. When cells were treated for 4 hours with either 1 μM or 10 μM of compound I-1, there was a significant restoration of the OCR upon the addition of FCCP, essentially to the same level observed with the normal control group and the positive control groups. Treatment of cells with compound I-1 resulted in a significant improvement in mitochondrial bioenergetics; and cells were able to cope with large external source of stress, induced by 500 μM of H₂O₂.

FIG. 1B essentially captures the data shown in FIG. 1A, in bar graph format, with the OCR data corrected for the amount that was inhibited by the addition of antimycin A. FIG. 1B shows how treatment of C2C12 cells with compound I-1 restored mitochondrial bioenergetics to about the same level observed with normal cells or cells that have been treated with catalase or NAC prior to being stressed with H₂O₂.

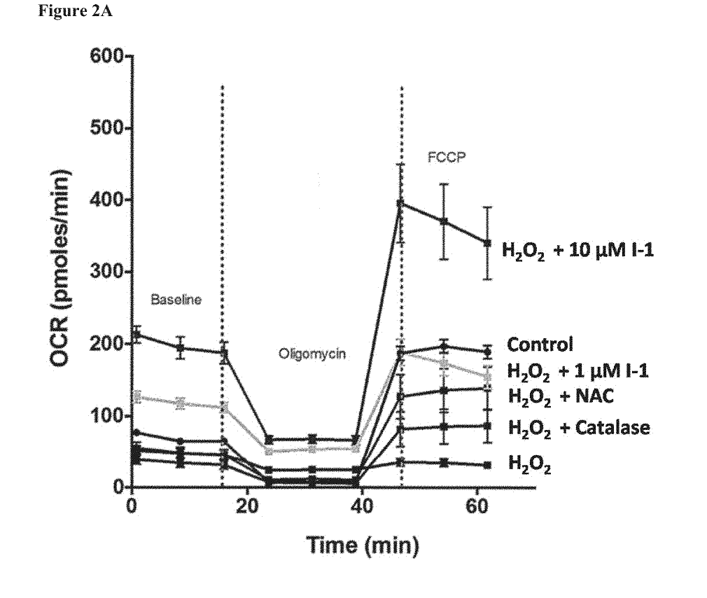

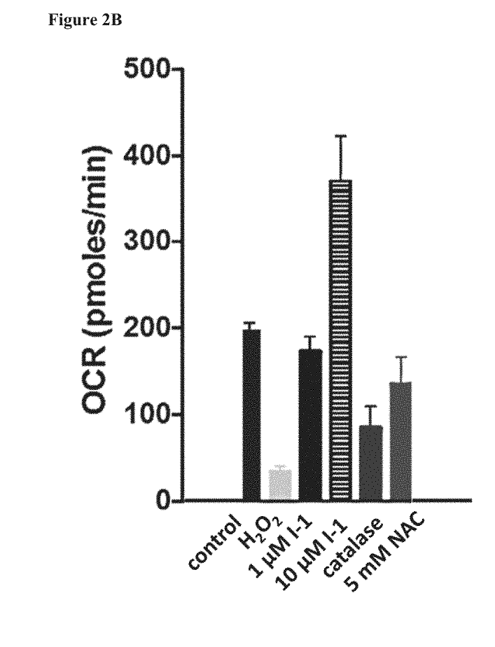

FIG. 2A shows the OCR data over time when C2C12 cells were treated for 24 hours with either 1) vehicle group; 2) 1 μM of compound I-1; 3) 10 μM of compound I-1; 4) positive control consisting of 5 mM of N-acetyl cysteine (NAC); 5) positive control consisting of 5K U/mL of the enzyme catalase. Each of these 5 treatment groups were exposed to a source of reactive oxygen species consisting of 500 μM of H₂O₂ as a stress. A 6^{th} treatment group, the “control group”, consisted of C2C12 cells that have been treated with the vehicle group but not exposed to 500 μM of H₂O₂. As shown in FIG. 2A, treatment with compound I-1 for 24 hours prior to the H₂O₂ stress showed a significant improvement in bioenergetics, and the effect was dose-dependent. The OCR over time was considerably higher for the treatment group consisting of 10 μM compound I-1, when compared with the “control group” and the two positive control groups. FIG. 2B summarizes the data shown in FIG. 2A, in bar graph format, and corrected for the addition of antimycin A. When C2C12 cells were stressed, the OCR decreased significantly (FIG. 2B, comparing the control group to the H₂O₂ treatment group). Treatment with 1 μM of compound I-1 restored the bioenergetics to the same level observed with the normal control group or those treated with catalase or NAC prior the H₂O₂ stress. When compared with the two positive control groups (catalase or NAC), treatment with 10 μM showed a substantial increase in mitochondrial bioenergetics, a nearly 4-fold increase in the OCR.

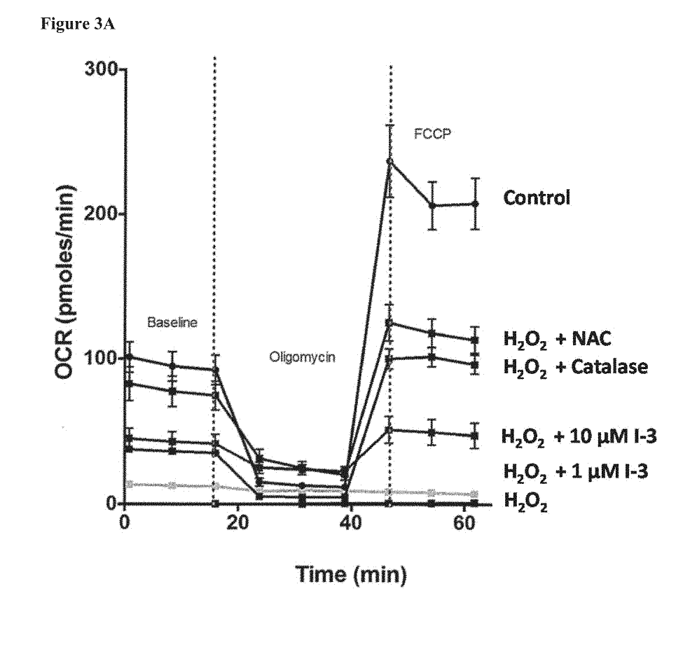

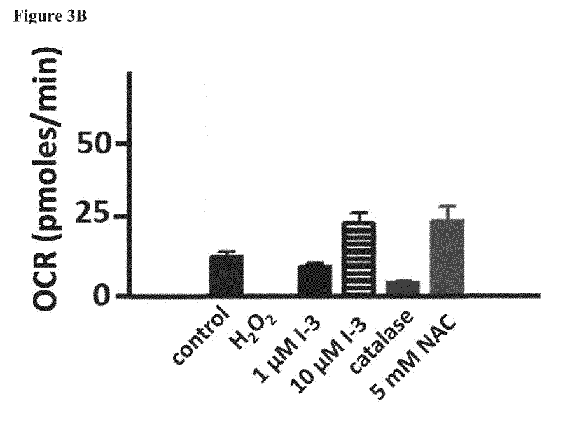

FIG. 3A shows the OCR data over time when C2C12 cells were treated for 4 hours with either 1) vehicle group; 2) 1 μM of compound I-3; 3) 10 μM of compound I-3; 4) positive control consisting of 5 mM of N-acetyl cysteine (NAC); 5) positive control consisting of 5K U/mL of the enzyme catalase. Each of these 5 treatment groups were exposed to a source of reactive oxygen species consisting of 1 mM of H₂O₂ as a stress. A 6^{th} treatment group, the “control group”, consisted of C2C12 cells that have been treated with the vehicle group but not exposed to 1 mM of H₂O₂. With a greater concentration of the stress agent H₂O₂, C2C12 cells did not display any respiration at the baseline level as well as upon the addition of FCCP. Treatment with the two positive controls (catalase or NAC) resulted in an increase in the OCR upon the addition of FCCP; almost to the same level observed with the normal control groups. Treatment with compound I-3 for just 4 hours prior to the 1 mM H₂O₂ stress resulted in a dose-dependent increase in bioenergetics, as shown in the increase in the OCR. FIG. 3B summarizes the data shown in FIG. 3A, in bar graph format, and corrected for the addition of antimycin A. The OCR corresponding to 10 μM of compound I-3 showed a significant improvement in mitochondrial bioenergetics, essentially comparable to the positive control NAC.

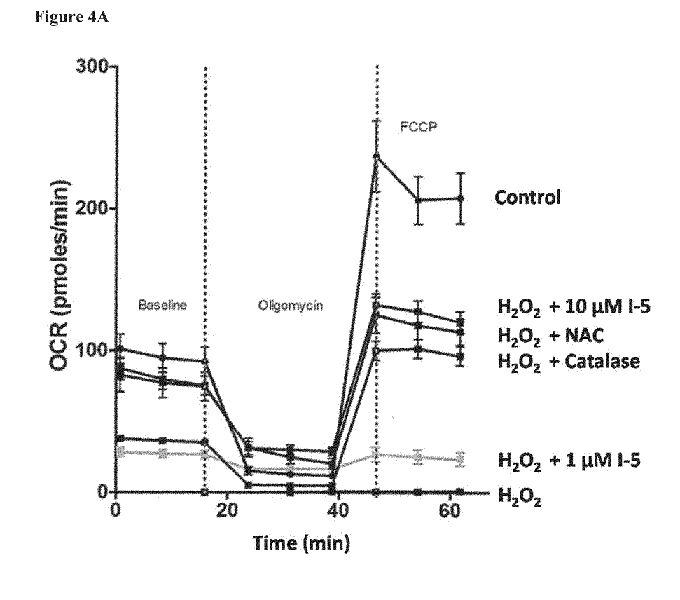

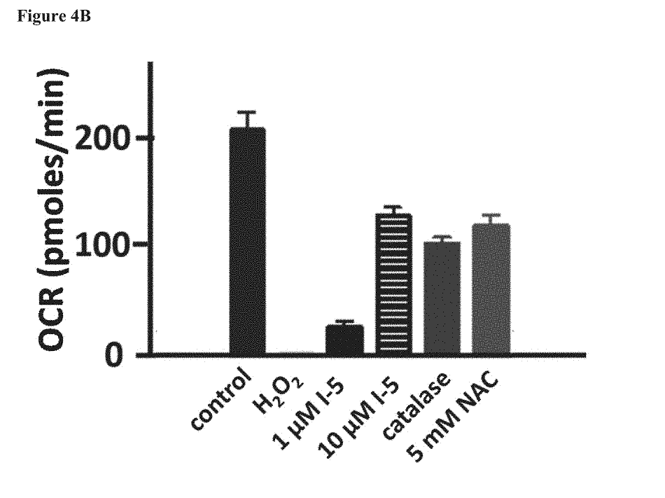

FIG. 4A shows the OCR data over time when C2C12 cells were treated for 4 hours with either 1) vehicle group; 2) 1 μM of compound I-5; 3) 10 μM of compound I-5; 4) positive control consisting of 5 mM of N-acetyl cysteine (NAC); 5) positive control consisting of 5K U/mL of the enzyme catalase. Each of these 5 treatment groups were exposed to a source of reactive oxygen species consisting of 1 mM of H₂O₂ as a stress. A 6^{th} treatment group, the “control group”, consisted of C2C12 cells that have been treated with the vehicle group but not exposed to 1 mM of H₂O₂. With a greater concentration of the stress agent H₂O₂, C2C12 cells did not display any respiration at the baseline level as well as upon the addition of FCCP. Treatment with the two positive controls (catalase or NAC) resulted in an increase in the OCR upon the addition of FCCP; almost to the same level observed with the normal control groups. Treatment with compound I-5 for just 4 hours prior to the 1 mM H₂O₂ stress resulted in a dose-dependent increase in bioenergetics, as shown in the increase in the OCR. FIG. 4B summarizes the data shown in FIG. 4A, in bar graph format, and corrected for the addition of antimycin A. The OCR corresponding to 10 μM of compound I-5 showed a significant improvement in mitochondrial bioenergetics, essentially comparable to the two positive control arms, catalase or NAC.

### Example 11

### Assay to Evaluate Nrf2 Activity

The transcription factor Nrf2 and its repressor Keap1 regulate a large network of antioxidant genes. The mitochondrial inner membrane is particularly susceptible to damage by reactive oxygen species. Antioxidants can play a large role in preserving the mitochondrial inner membrane and maintaining the integrity of the respiratory chain. Holmstrom et al. (BIOLOGY OPEN (2013) 2, p. 761-770) have also shown that the Nrf2 pathway can potentially control the NADH substrates that are needed for mitochondrial respiration. Activation of the Nrf2 pathway is a way of improving the mitochondrial bioenergetics. The compounds of the invention were evaluated in an assay that was designed to assess the various antioxidant genes that could be regulated by the transcription factor Nrf2.

Briefly, mouse C2C12 cells were treated with the test compounds when about 70% of the myotubes were differentiated (Day 13). For the assay, cells were treated with the test compounds for 24 hours in low glucose, 0.5% FBS DMEM and 0.1% BSA final concentration. The total RNA was harvested and the relative mRNA expression levels were assessed via RT-PCR with Hypoxanthine phosphoribosyltransferase (“HPRT”) as the internal control. Data were represented as the mean ΔmRNA/HPRT, error bars represent the standard error of the mean (SEM). Significance was determined by student's t-test in comparison to control. The Nrf2 gene expression targets include:

Gclc—Glutamate-cysteine ligase catalytic subunit;
Gclm—Glutamate-cysteine ligase modifier subunit;

### Gsta2—Glutathione S-transferase A2;

HMOX1—Heme oxygenase 1;
NQO1—NAD(P)H dehydrogenase (quinone) 1;

### SRXN1—Sulfiredoxin-1;

TXNRD1—Thioredoxin reductase 1.

To those familiar in the art, an increase in the mRNA level of any of these gene expression targets indicates that the transcription factor Nrf2 has been up-regulated. Compound I-1 was evaluated in this assay for any upregulation in the seven Nrf2 gene expression targets listed above.

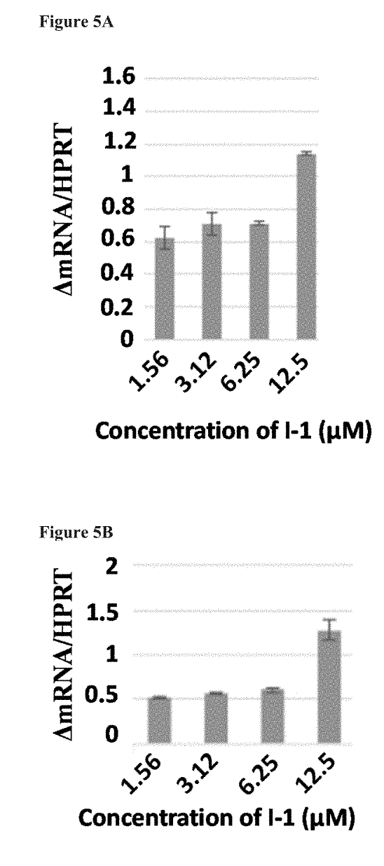

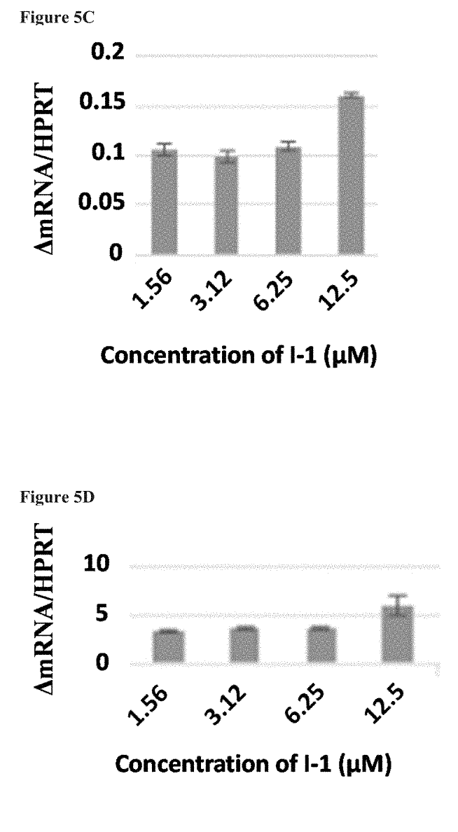

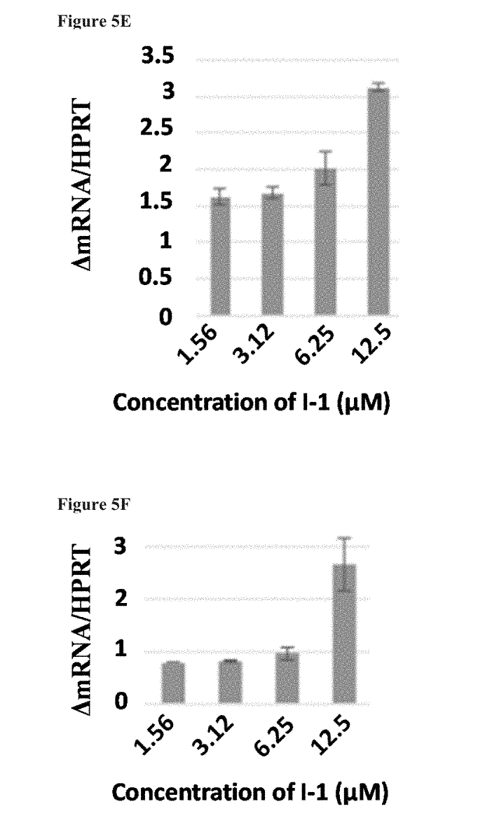

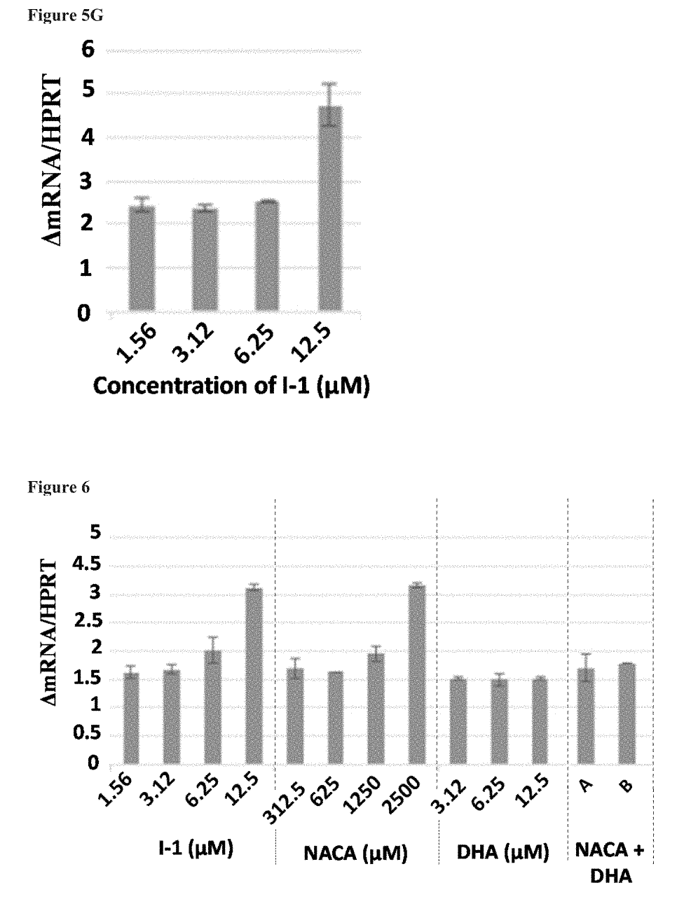

FIG. 5A shows the ΔmRNA/HPRT level for the Gclc gene vs concentration of compound I-1. No significant increase of mRNA expression level of Gclc was observed at the lower concentrations of 1.56 to 6.25 μM of compound I-1. At 12.5 μM of compound I-1, there was a significant increase in the expression of mRNA for the Gclc gene.

FIG. 5B shows the ΔmRNA/HPRT level for the Gclm gene vs concentration of compound I-1. No significant increase of mRNA expression level of Gclm was observed at the lower concentrations of 1.56 to 6.25 μM of compound I-1. At 12.5 μM of compound I-1, there was a significant increase in the expression of mRNA for the Gclm gene.

FIG. 5C shows the ΔmRNA/HPRT level for the Gsta2 gene vs concentration of compound I-1. No significant increase of mRNA expression level of Gsta2 was observed at the lower concentrations of 1.56 to 6.25 μM of compound I-1. At 12.5 μM of compound I-1, there was a significant increase in the expression of mRNA for the Gsta2 gene.

FIG. 5D shows the ΔmRNA/HPRT level for the HMOX gene vs concentration of compound I-1. No significant increase of mRNA expression level of HMOX was observed at the lower concentrations of 1.56 to 6.25 μM of compound I-1. At 12.5 μM of compound I-1, there was a significant increase in the expression of mRNA for the HMOX gene.

FIG. 5E shows the ΔmRNA/HPRT level for the NQO1 gene vs concentration of compound I-1. No significant increase of mRNA expression level of Gclc was observed at the lower concentrations of 1.56 to 3.12 μM of compound I-1. At the 6.25 μM concentration of compound I-1, there was an increase in the expression of mRNA for the NQO1; and at 12.5 μM of compound I-1, there was a two-fold increase in the expression of mRNA for the NQO1 gene.

FIG. 5F shows the ΔmRNA/HPRT level for the SRXN1 gene vs concentration of compound I-1. No significant increase of mRNA expression level of SRXN1 was observed at the lower concentrations of 1.56 to 6.25 μM of compound I-1. At 12.5 μM of compound I-1, there was a significant increase in the expression of mRNA for the SRXN1 gene.

FIG. 5G shows the ΔmRNA/HPRT level for the TXNRD1 gene vs concentration of compound I-1. No significant increase of mRNA expression level of TXNRD1 was observed at the lower concentrations of 1.56 to 6.25 μM of compound I-1. At 12.5 μM of compound I-1, there was a significant increase in the expression of mRNA for the TXNRD1 gene.

FIG. 6 shows the results for a synergy experiment for the fatty acid cysteine conjugate I-1. The ΔmRNA/HPRT level for NQO1 was selected for this experiment. As discussed earlier, compound I-1 induced a dose-dependent increase in the expression of mRNA of NQO1 and a maximal response was observed at 12 μM. With this covalent conjugate, the two key bioactive components were the N-acetylcysteine amide (NACA) and the omega-3 fatty acid. Previous work (Patel et al, Experimental Neurology 2014, 257, p. 95-105) has shown that NACA could upregulate Nrf2 gene expression and consequently mitochondrial bioenergetics. However, the effect was modest and a high concentration of NACA was needed. We confirmed that result and as shown in FIG. 6, NACA could only begin to upregulate the expression of mRNA for the NQO1 gene at the high concentration of 1250 μM. In order to obtain the same level of activity as 12 μM of the fatty acid cysteine conjugate I-1, a much higher concentration of 2,500 μM of NACA was needed. In this assay, the omega-3 fatty acid DHA began to show signs of toxicity at concentration of >25 μM. As shown in FIG. 6, we tested DHA at up to 12 μM and did not observe any upregulation in the mRNA level of the NQO1 gene. In terms of combination of the individual components, the combination of A (consisting of 312.5 μM of NACA and 3.12 μM of DHA) and the combination of B (consisting of 625 μM of NACA and 6.25 μM of DHA) were used. Both were ineffective and did not increase the expression of mRNA level of the NQO1 gene. The fatty acid cysteine conjugate I-1 was able to increase the Nrf2 pathway synergistically and the effect could not be replicated by the administering the individual components or a combination of the individual components.

### Example 12

### Effect of Compounds on Autophagy, CFTR in Huh-7, HT-29 or THP-1 Cells

The compounds of the invention are evaluated for their effect on autophagy and CFTR trafficking using the following procedure.

Compound Preparation:

Test compounds are first solubilized in 100% DMSO as 50 mM solution, and then diluted 1 to 100 in FBS as a 10× stock solution of 500 μm.

Immunoblotting:

Huh-7 or HT-29 or THP-1 cells are seeded in 10% FBS DMEM overnight. The cells media is replaced with drug diluted 1 to 10 in DMEM (final concentration 50 μm in 10% FBS DMEM). 24 hours after the drug addition, cells are lysed in RIPA buffer. Cell lysates are analyzed by immunoblotting with anti-LC3A/B antibodies (Cell signaling 12741) and anti-CFTR antibodies (Cell signaling 2269). Data of LC3 are presented as LC3-II/LC3-I ratio compared to vehicle treated samples. Data of CFTR are normalized with actin and represented as ratio compared to the vehicle treated samples.

Cell Surface Biotinylation:

HT-29 cells are seeded at 2.0×10⁶ cells in 10 mm² plates in 10% FBS DMEM overnight. The cells media are replaced with drug diluted 1 to 10 in DMEM (final concentration 50 μm in 10% FBS DMEM). 24 hours after the drug addition, cells are processed for cell surface biotinylation using Pierce Cell Surface Protein Isolation Kit (Thermo Scientific 89881). Briefly, cells are washed once with cold PBS and incubated with Sulfo-NHS-SS-Biotin for 30 minutes at 4° C. and reaction is stopped by adding Quenching solution. Cells are scraped and lysed and centrifuged at 10,000×g. Cell suspension are incubated with NeutrAvidin Agarose for 60 minutes at room temperature. Cell pellet (intracellular) are lysed in RIPA buffer. Protein bound to NeutrAvidin Agarose (cell surface) are eluted by SDS-PAGE sample buffer containing 50 mM DTT. Both cell surface and intracellular parts are analyzed by immunoblotting with anti-CFTR antibodies.

### Example 13

### Effect of Compounds of the Invention on Primary CF Human Bronchial Epithelial Cells, Homozygous for ΔF508 CFTR Deletion Mutation: Immunoblot Analysis and Immunoprecipitation

It is contemplated that compounds of the invention may be useful in treating CF given their ability to activate autophagy. The compounds of the invention are evaluated in the following cellular assay to determine their ability to traffic the defective, mutant CFTR to the cell membrane.

Primary cells from homozygous ΔF508 CF patients can be obtained from either Asterand Bioscience (Detroit, Mich.) or ChanTest, a Charles River Company (Cleveland, Ohio). Cells are then treated at various concentrations to determine the compound's ability to restore the defective CFTR. As reviewed in Derichs (2013) EUR. RESP. REV., 22, p. 58-65, a successful detection of the mutant CFTR band C by immunoblot indicates that the defective CFTR can be rescued to the cell membrane.

Compound Preparation:

Compounds of the invention are first solubilized in 100% DMSO as 50 mM solution, and then diluted 1 to 100 in FBS as a 10× stock solution of 500 μM.

Immunoblotting:

Primary CF cells (homozygous ΔF508, source: ChanTest, KKCFFT004I) are prepared and grown on Snapwell™ filter inserts according to the procedures outlined in Amaral, M. D. and Kunzelmann, K. (Eds) (2011) CYSTIC FIBROSIS, METHODS IN MOLECULAR BIOLOGY 741, DOI 10.1007/978-1-61779-117-8_4 Springer Science+Business Media, LLC). Primary CF cells are kept in differentiation media consisting of Dulbecco's MEM (DMEM)/F12, Ultroser-G (2.0%; Pall, Catalog #15950-017), fetal clone II (2%), insulin (2.5 μg/ml), bovine brain extract (0.25%; Lonza, Kit #CC-4133, component # CC-4092C), hydrocortisone (20 nM), triiodothyronine (500 nM), transferrin (2.5 μg/ml: Invitrogen, Catalog #0030124SA), ethanolamine (250 nM), epinephrine (1.5 μM), phosphoethanolamine (250 nM), and retinoic acid (10 nM). The test compounds, solubilized in FBS according to the procedure outlined above and diluted to the desired concentration, are then added to the individual Snapwell™ filter inserts in the differentiation media at 37° C. Twenty-four hours after the drug addition, cells are snap frozen and later lysed in RIPA buffer. The amounts of proteins are determined by Bio-Rad protein assay. Fifty μg of total cell lysates are analyzed by immunoblotting with anti-CFTR, anti-Beclin-1, anti-p62 and anti-LC3 antibodies. The immuno-activity can be normalized with actin as the loading control. Data can be presented as CFTR-band-C/actin, Beclin-1/actin, p62/actin and LC3-II/LC3-I ratio compared to vehicle treated samples. Antibodies against CFTR clone M3A7 (Cell Signaling Technology, 2269), LC3A/B antibodies (Cell signaling, 12741), Beclin-1 (Cell Signaling Technology, 3495), p62 (Cell Signaling Technology, 5114) and β-actin (Cell Signaling Technology, 4970) are used as primary antibodies.

### Example 14

### Evaluation of Compounds in the Fisher Rat Thyroid Epithelial Cells Ussing Chamber for Functional Rescue of CFTR Ion Channel Activity.

The most prevalent disease causing mutation of the CF transmembrane conductance regulator (CFTR) chloride channel is deletion of phenylalanine at position 508 in the primary sequence of CFTR (ΔF508-CFTR). This mutation causes a trafficking defect resulting in a severe reduction of ΔF508-CFTR protein at the cell surface. The trafficking defect can be corrected by incubation at low temperature (27° C. overnight) or pharmacologically by small molecules and CFTR correctors. Chloride transport function of Fisher Rat Thyroid (FRT) epithelial cells overexpressing ΔF508-CFTR in monolayers grown on Snapwell™ filter inserts are monitored as the CFTR agonist evoked short circuit (I_{SC}) current output of an Ussing epithelial voltage clamp apparatus. An objective of this study is to measure the ability of test compounds to restore function to defective ΔF508-CFTR in FRT epithelial cell monolayers.

Measurement of corrector efficacy can be divided into two phases. The initial phase is incubation of epithelia with the test compounds for a period of time (that can range from 2 hours to one or two days) in a 37° C. incubator and the second phase is measurement of epithelial ΔF508-CFTR chloride channel current with an epithelial voltage clamp (Ussing assay). The short circuit current (I_{SC}) is measured under short circuit conditions (0 mV transepithelial potential). The I_{SC} magnitude is an index of corrector efficacy and is compared to vehicle and positive control.

Cryopreserved FRT cells stably transfected with ΔF508-CFTR cDNA (Pedemonte et al. (2005) J. CLIN. INVEST., 115, p. 2564-2571) are expanded and plated on Snapwell™ filters for measurement of short circuit current in an Ussing apparatus (Physiologic Instruments, Inc., Sand Diego, Calif.). Cells are grown in Coon's modification of Ham's F-12 media supplemented with zeocin and G-418.

To conduct the assay, a compound of the invention is solubilized as follows:
1) Prepare 25 mM stock solution in 100% DMSO.
2) Dilute 12 μL of 25 mM DMSO stock in 1.20 mL of FBS to make 250 μM intermediate dilution 10× stock (1% DMSO, 99% FBS), and gently vortex until they become clear.
3) Prepare final dilution of 10 μM in 10 mL of Coon's media per well (4 wells×2 mL per well bottom+0.2 mL per insert top=8.8 mL and 1.2 mL reserve for handling losses) by addition of 400 μL of 10× stock and 600 μL of 10× carrier (1% DMSO, 99% FBS) to 9000 μL of Coon's media.

For the following example, the test compound is solubilized and added to the appropriate inserts of Us sing chambers (n=4 for each test compound, final test concentration of 10 μM). A DMSO vehicle control and a positive control (VX-809 at 3 μM) are also used. For this particular example, all the test articles, including the positive control, are incubated with the cells for a period of 4 hours. The FRT cell monolayers grown on Snapwell™ filter inserts are transferred to Physiologic Instruments Ussing recording chambers (Physiologic Instruments, Inc., San Diego, Calif.) and superfused with HB-PS on the basolateral side and 78CF-PS on the apical side. One or more 6-channel or 8-channel Physiologic Instruments VCC MC6 or VCC MC8 epithelial voltage clamps are then used in combination to record the short circuit current (I_{SC}) during the entire run. To initiate the I_{SC} measurement, amphotericin (100 μM) was added to the basolateral side of the Snapwell™ filter insert to permeabilize the epithelial cells for 15 min. Forskolin (10 μM), IBMX (100 μM), Genistein (20 μM) and the CFTRᵢₙₕ-172 (20 μM) are added sequentially after the following incubation periods (15 min, 20 min, 10 min, 15 min and 15 min respectively). Data acquisition and analyses are performed using iWorx data acquisition hardware and Labscribe 2 software (iWorx, Dover, N.H.). Comparison of agonist evoked I_{SC} among both corrector positive control, negative control and test article treated epithelia is obtained with one-way ANOVA followed by Dunnett's multiple comparison test and Student's t-test when appropriate. Significant correction is defined at the level of P<0.05.

In this assay, the positive control VX-809 is able to functionally rescue the defective CFTR under the test conditions when the cells were treated sequentially with Forskolin, IBMX and then with the CFTR potentiator Genistein. To test for CFTR specificity, the commercially available inhibitor CFTRᵢₙₕ-172 (which has chemical name (E)-4-((4-oxo-2-thioxo-3-(3-(trifluoromethyl)phenyl)thiazolidin-5-ylidene)methyl)benzoic acid (CAS no. 307510-92-5)) is added near the completion of the run to bring the short circuit current down to the baseline.

An alternative protocol to this assay involves the chronic pre-incubation of compounds of the invention along with VX-770 or the combination of VX-809 and VX-770. With this protocol, the compounds of the invention are pre-incubated with either VX-770 or a combination of VX-770 and VX-809 for 24 hours using the same protocols outlined above. Amphotericin (100 μM) is first added to permeabilize the cell membrane. Fifteen minutes after the addition of amphotericin, Forskolin (20 μM) is added. Twenty minutes after the addition of Forskolin, the commercially available inhibitor CFTRᵢₙₕ-172 is added. The reaction is then terminated 15 minutes after the addition of the CFTRᵢₙₕ-172. A representative trace of the short circuit currents is then obtained from this type of experiment. The functional activity of the compounds of the invention is assessed when comparison was made between the vehicle group and the positive control group. For all the Ussing chamber experiments, the positive control was a combination of the CFTR corrector VX-809 (3 μM) and the CFTR potentiator VX-770 (100 nM). Quantification of the short circuit currents was carried out to determine the ΔI_{SC} at two different time points, first upon the addition of Forskolin and later upon the addition of the CFTRᵢₙₕ-172 (For a more comprehensive description of the assay, please refer to Van Goor et al. (2011) PNAS, 108 (46), p. 18843-18848).

### Example 15

### Evaluation of Compounds in the Primary CF Bronchial Epithelial Cells (Homozygous F508) Ussing Chamber for Functional Rescue of CFTR Ion Channel Activity

Primary CF cells (homozygous ΔF508, source: ChanTest, KKCFFT004I) are prepared and grown on Snapwell™ filter inserts according to the procedures outlined in Amaral, M. D. and Kunzelmann, K. (Eds) (2011) CYSTIC FIBROSIS, METHODS IN MOLECULAR BIOLOGY 741, DOI 10.1007/978-1-61779-117-8_4 Springer Science+Business Media, LLC). Primary CF cells are kept in differentiation media consisting of Dulbecco's MEM (DMEM)/F12, Ultroser-G (2.0%; Pall, Catalog #15950-017), fetal clone II (2%), insulin (2.5 μg/ml), bovine brain extract (0.25%; Lonza, Kit #CC-4133, component # CC-4092C), hydrocortisone (20 nM), triiodothyronine (500 nM), transferrin (2.5 μg/ml: Invitrogen, Catalog #0030124SA), ethanolamine (250 nM), epinephrine (1.5 μM), phosphoethanolamine (250 nM), and retinoic acid (10 nM).

To run the Us sing chamber assay, the test compounds (solubilized in FBS according to the procedure outlined above, and diluted to the desired concentration) are then added to the individual Snapwell™ filter inserts in the differentiation media at 37° C. Twenty-four hours after the drug addition, the inserts are transferred to Physiologic Instruments Us sing recording chambers (Physiologic Instruments, Inc.; San Diego, Calif.) and maintained in both the apical and basolateral chambers with a HEPES buffered physiological saline (HB-PS) with composition (in mM): NaCl, 137; KCl, 4.0; CaCl₂, 1.8; MgCl₂, 1; HEPES, 10; Glucose, 10; pH adjusted to 7.4 with NaOH. One or more 6-channel or 8-channel Physiologic Instruments VCC MC6 or VCC MC8 epithelial voltage clamps are then used in combination to record the short circuit current (I_{SC}) during the entire run. The short circuit I_{SC} measurements are conducted at 27° C. To initiate the run, amiloride (30 μM) is added to the apical side of the Snapwell™ filter inserts to block epithelial Na channels (ENaC). Fifteen minutes later, Forskolin (10 μM) is added to activate the CFTR. Sixty minutes later, the experiment is terminated by the addition of the CFTRᵢₙₕ-172 (20 μM). Data acquisition and analyses are performed using iWorx data acquisition hardware and Labscribe 2 software (iWorx, Dover, N.H.). Comparison of agonist evoked I_{SC} among both corrector positive control, negative control and test article treated epithelia is obtained with one-way ANOVA followed by Dunnett's multiple comparison test and Student's t-test when appropriate. Significant correction is defined at the level of P<0.05.

### Example 16

### In Vitro Bacterial Clearance Assay Using Human Bronchial Epithelial Cells

In this assay, normal 16HBE cells are cultured and seeded at 2×10⁵ cells per well using a 48-well plate. The resulting plates are incubated at 37° C. with 5% CO₂ until ˜90% confluency. Cells are then treated with the vehicle control, the desired test compound and a positive control (Cytochalasin-D) for 24 hours and then infected with Pseudomona aeruginosa strain Xen05 at a multiplicity of infection (MOI) of 1:50 (i.e. ratio of cells:bacteria) for 2 hours. Cells then are incubated with 500 μL of a mixture consisting non-permeable antibiotic (50 U/mL each of pencillin and streptomycin, mixed with 200 μg/mL gentamicin) for 3 hours to remove the extracellular bacteria. Afterwards, cells are lysed and a bacteria count is carried out to determine the remaining intracellular bacteria load.

### Example 17

### Assay to Assess Plasma Stability of Fatty Acid Cysteine-Based Conjugates

The in vitro stability of the test compounds can be studied in human, mouse, beagle and rat plasma (Plasma was purchased from Bioreclamation). Plasma is diluted to 50% with PBS (pH 7.4). Test compounds are dissolved in DMSO to a final concentration of 10 mM and then diluted to 1 mM in MeOH. Incubations are carried out at a test compound concentration of 5 μM with a final DMSO concentration of 2.5%. Plasma (198 μL) is added to 96-well plate and incubated at 37° C. for 30 minutes before the addition of 2 μL of the test compound. The resulting mixture is then incubated at 37° C. for 2 hours. At appropriate time intervals (0, 30, 60 and 120 min), aliquots (50 μL) are removed and reactions were terminated by adding 200 μL of acetonitrile with an internal standard. Simultaneously, plasma samples containing Benflourex or Procaine (control compound) are terminated by adding 200 μL of acetonitrile internal standard. The sample plate is centrifuged at 3500 rpm for 45 minutes at 4° C. and the supernatant is transferred to a new plate for analysis by LC/MS-MS (Agilent Model No: HPLC: 1200, MS: 6410). Chromatographic separation is achieved with a Phenomenex C6-phenyl (5u) column. A binary gradient consisting of 0.1% formic acid in water and 0.1% formic acid in methanol is used for analyte elution.

### Example 18

### Evaluation of Fatty Acid Cysteine-Based Conjugates in an Oral Cannulated Rat PK Study

The compounds of the invention can be solubilized in a mixture of excipients consisting 40% Tween, 50% Peceol, 10% PEG400 and diluted with water to form a self-emulsifying aqueous mixture for oral administration to animals. For this study, Sprague Dawley rats that are surgically implanted with indwelling jugular vein cannula (JVC) and portal vein cannula (PVC) can be used (Agilux, Worcester, Mass.). This approach using double-cannulated rats allows the measurement of the drug concentration that is delivered in the portal vein as well as the drug concentration that is present in the peripheral. For the PK study, serial blood collection is carried out at both the portal and jugular vein at the following time points: 10, 20, 40 minutes and 1, 2, 4 and 6 hours post dose. The bioanalytical portion of the PK study is carried out using an LC/MS/MS system (Agilent Model No: HPLC: 1200, MS: 6410) and analyzed with the appropriate software (WinNonlin Phoenix 64 6.3.0 395).

### Example 19

### In Vivo Determination of Autophagy Activation

In order to evaluate for in vivo autophagy activation, naïve male C57BL/6 mice are dosed orally with the desired test compound (100 mg/kg, BID, 3.5 days). One hour after the last dose, lung tissues and plasma are collected to analyze for drug concentration and autophagy biomarkers. As discussed in earlier examples, the ratio of LC3-II to LC3-I, obtained from lung tissues, is used as autophagy biomarker.

### Example 20

Assessment of Fatty Acid Cysteine Conjugate in a Model of Murine Lung Infection with Pseudomonas aeruginosa

In this model of murine lung infection with Pseudomonas aeruginosa, female BALB/c mice, aged 6-7 weeks, are allowed to acclimate for one week in five groups of 10 animals per cage. From 3.5 days prior to the infection, animals are treated with the test compound (formulated as described above) at 100 mg/kg po, BID; animals were then kept on the same compound treatment for the duration of the study. Four other treatment groups are used in this study, including the vehicle control and the positive control groups: Group 1) vehicle, po (BID from day −3.5) and s.c. (BID from 8 hours post infection); Group 2) test compound, po (BID, 100 mg/kg from day −3.5) plus vehicle s.c. (BID from 8 hours post infection); Group hours hours r post infection), plus vehicle p.o. (BID from day −3.5); Group 4) Ciprofloxacin, 5 mg/kg s.c. (BID from 8 hours post infection), plus test compound po (BID, 100 mg/kg, from day −3.5); Group 5) Ciprofloxacin, positive control, 20 mg/kg s.c. (BID from 8 hours post infection).

Animals are weighed prior to treatment and daily thereafter until the termination of the study. Once infected with Pseudomonas aeruginosa, animals are observed regularly for signs of ill-health and body temperatures were monitored. Animals reaching humane endpoints are terminated and time of death recorded. At termination, 24/48 hours post infection, lungs are removed and signs of gross pathology scored and photographed. Lung, spleen, and kidney are removed, weighed and transferred into PBS, homogenized and serial dilutions plated out to determine the bacterial load.

The following is non-limiting list of embodiments contemplated by the invention.
I. A compound of Formula I:

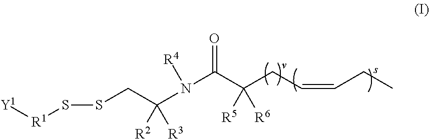

or a pharmaceutically acceptable salt or solvate thereof; wherein:

R¹ is C₁-C₆ alkylene optionally substituted by —C(O)N(R²)(hydroxyalkyl), —N(R²)C(O)-(hydroxyalkyl), —O-(hydroxyalkyl), —N(R²)-(hydroxyalkyl), —OC(O)N(R²)(hydroxyalkyl), —C(O)N(R²)(R⁸), —N(R²)C(O)R⁹, or —CO₂R⁹;

R³ is

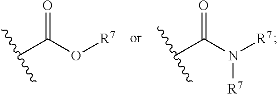

R², R⁴, and R⁷ each represent independently for each occurrence hydrogen or C₁-C₄ alkyl;

R⁵ and R⁶ are independently hydrogen, C₁-C₄ alkyl, or halogen;

R⁸ is C₁-C₆ alkyl, phenyl, benzyl, C₁-C₆ alkylene-CO₂R⁷, or C₁-C₆ alkylene-C(O)N(R⁷)₂;

R⁹ represents independently for each occurrence hydrogen, C₁-C₆ alkyl, phenyl, benzyl, C₁-C₆ alkylene-CO₂R⁷, or C₁-C₆ alkylene-C(O)N(R⁷)₂;

s is 3, 5, or 6;

v is 1, 2, or 6;

Y¹ is an amide or carbamate selected from the group consisting of —N(R⁷)C(O)-aralkyl, —N(R⁷)C(O)-(hydroxyalkyl), —C(O)N(R⁷)(hydroxyalkyl), —N(R⁷)CO₂-(hydroxyalkyl), —OC(O)N(R⁷)(hydroxyalkyl), —N(R⁷)C(O)—Z¹, and

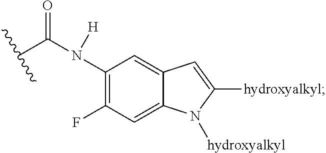

and

Z¹ is one of the following:

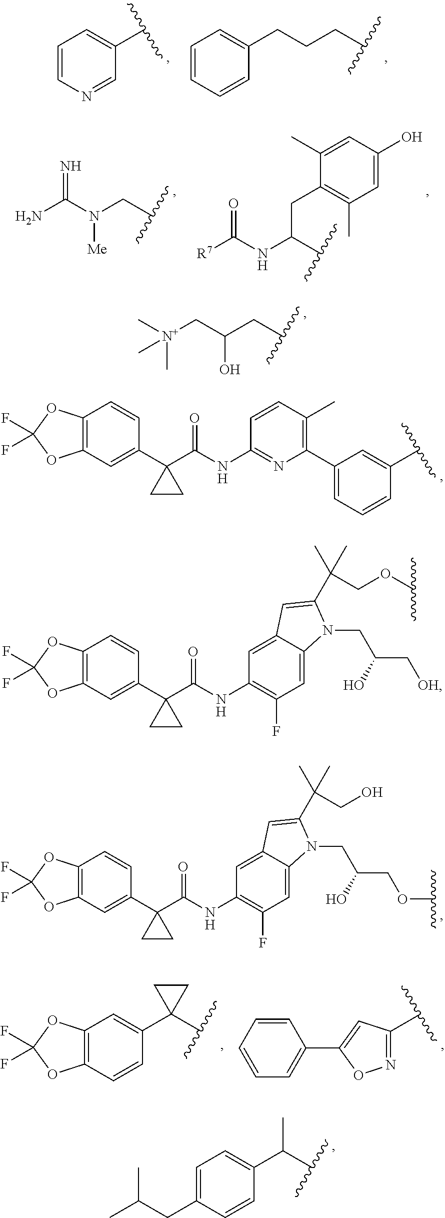

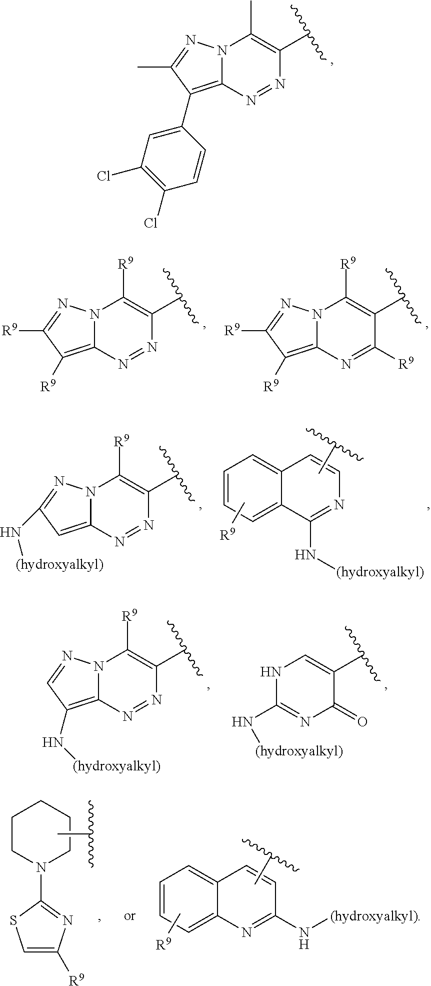

II. The compound of embodiment I, wherein R¹ is C₁-C₆ alkylene optionally substituted with —C(O)N(R²)(hydroxyalkyl), —N(R²)C(O)(hydroxyalkyl), —O-(hydroxyalkyl), —N(R²)-(hydroxyalkyl) or —OC(O)N(R²)(hydroxyalkyl).
III. The compound of embodiment I, wherein R¹ is C₁-C₆ alkylene.
IV. The compound of embodiment III, wherein R¹ is ethylene.
V. The compound of embodiment I or II, wherein R¹ is C₂-C₄ alkylene optionally substituted with one of the following:

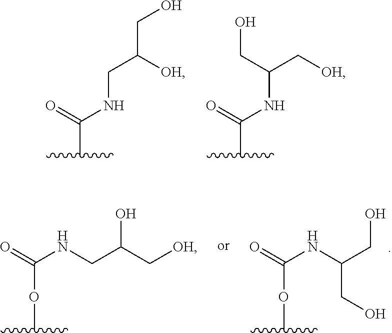 [SMILES: *C(=O)NCC(O)CO ; *C(=O)NC(CO)CO ; *OC(=O)NCC(O)CO ; "or" ; *OC(=O)NC(CO)CO ; "."]

VI. The compound of any one of embodiments I-V, wherein R2 and R4 are hydrogen.
VII. The compound of any one of embodiments I-VI, wherein R5 and R6 are hydrogen.
VIII. The compound of any one of embodiments I-VII, wherein R⁷ is hydrogen.
IX. The compound of any one of embodiments I-VIII, wherein R³ is

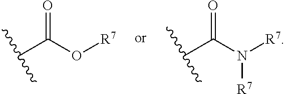

X. The compound of any one of embodiments I-IX, wherein v is 1, and s is 6.
XI. The compound of any one of embodiments I-IX, wherein v is 2, and s is 5.
XII. The compound of any one of embodiments I-XI, wherein Y¹ is —N(R⁷)C(O)-aralkyl.
XIII The compound of any one of embodiments I-XI, wherein Y¹ is —N(R⁷)C(O)—(CH₂)₃-phenyl.
XIV. The compound of any one of embodiments I-XI, wherein Y¹ is —N(R⁷)C(O)-(hydroxyalkyl), —C(O)N(R⁷)(hydroxyalkyl), —N(R⁷)CO₂-(hydroxyalkyl), or —OC(O)N(R⁷)(hydroxyalkyl).
XV. The compound of any one of embodiments I-XI, wherein Y¹ is selected from:

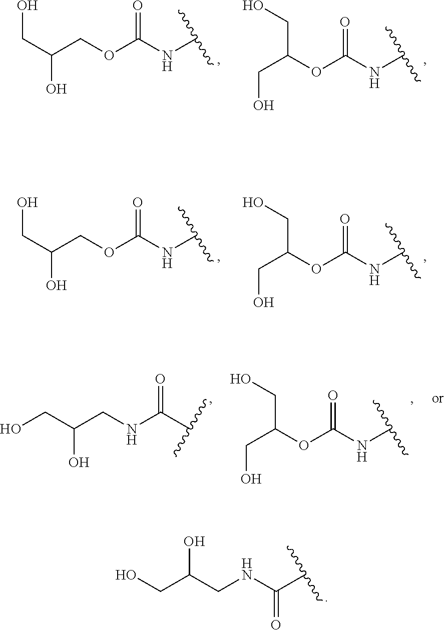

XVI. The compound of any one of embodiments I-XI, wherein Y¹ is —N(R⁷)C(O)—Z¹.
XVII. The compound of any one of embodiments I-XI or XVI, wherein Z¹ is selected from the group consisting of

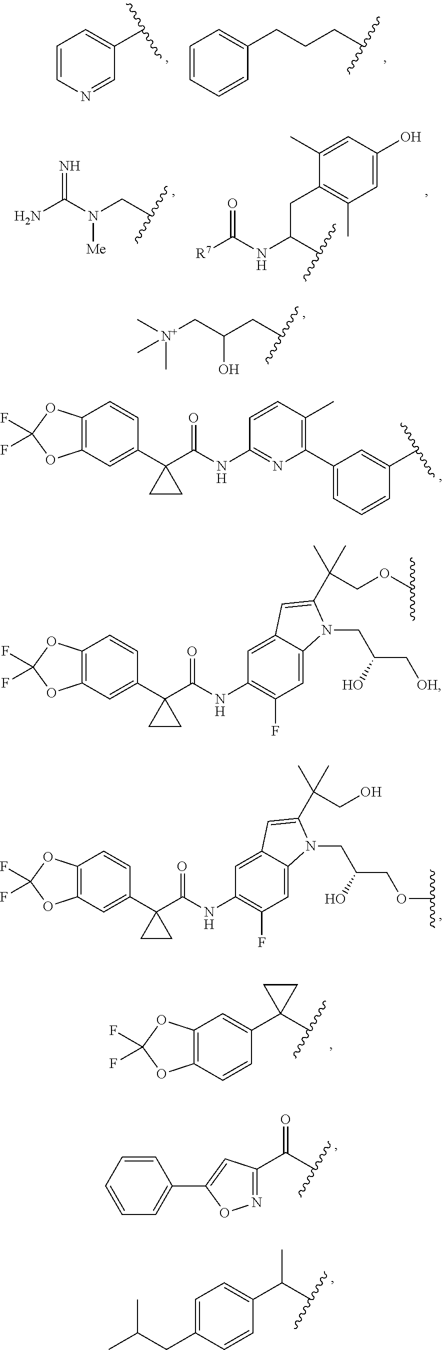

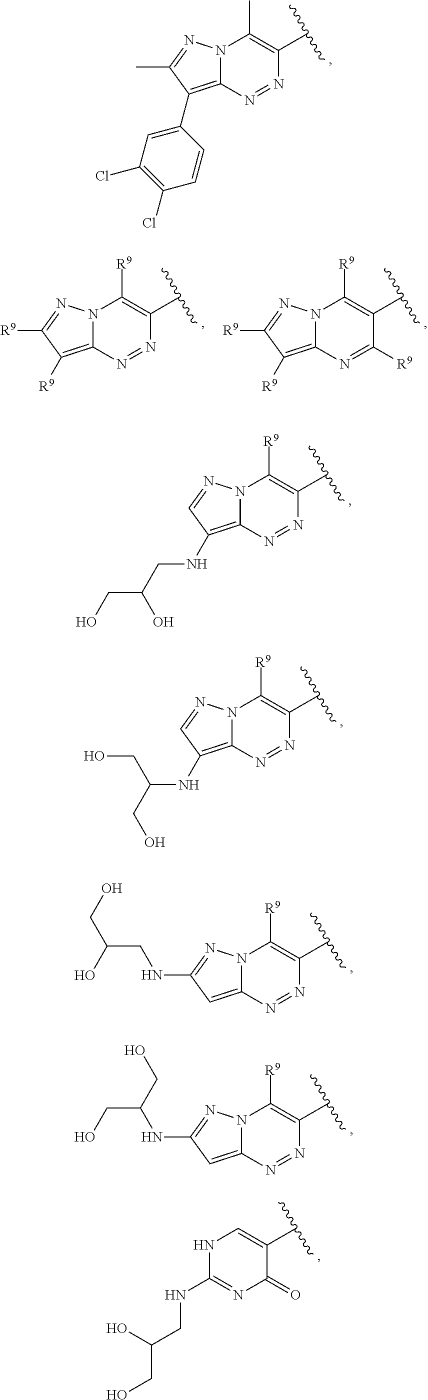

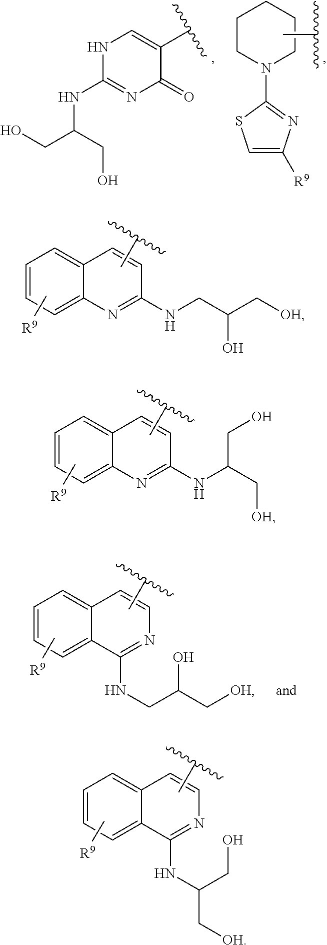

XVIII. The compound of any one of embodiments I-XI, wherein Y¹ is

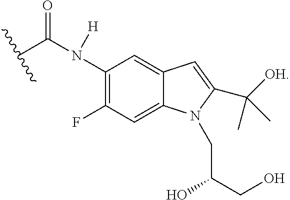 [SMILES: *C(=O)Nc1cc2cc(C(C)(C)O)n(C[C@@H](O)CO)c2cc1F]

XIX. A compound of Formula II:

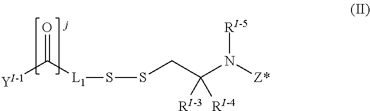

or a pharmaceutically acceptable salt or solvate thereof; wherein:

L₁ is independently selected from the group consisting of

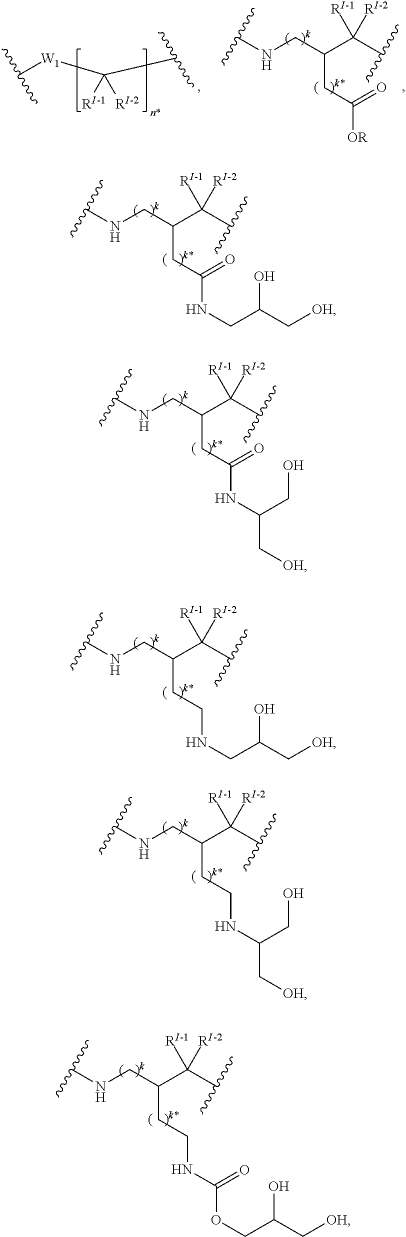

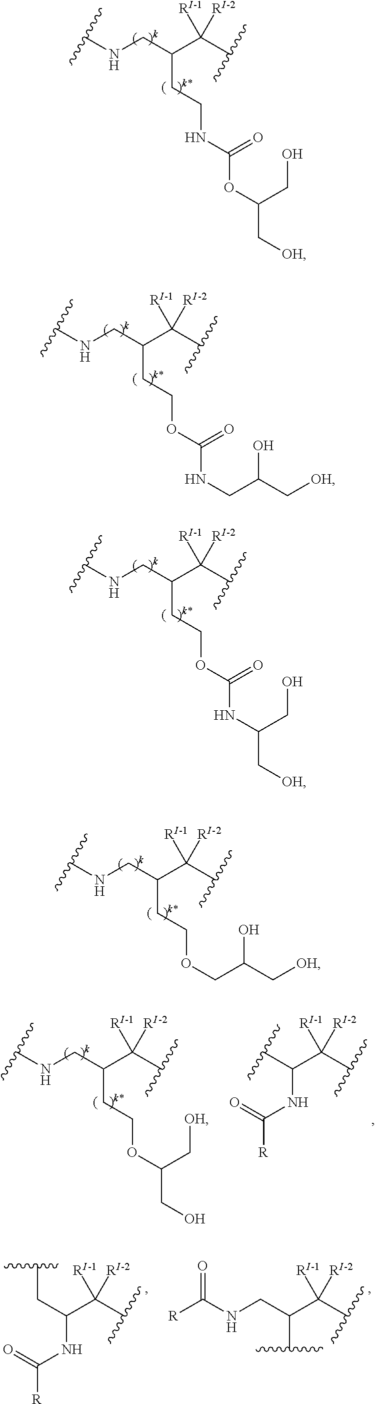

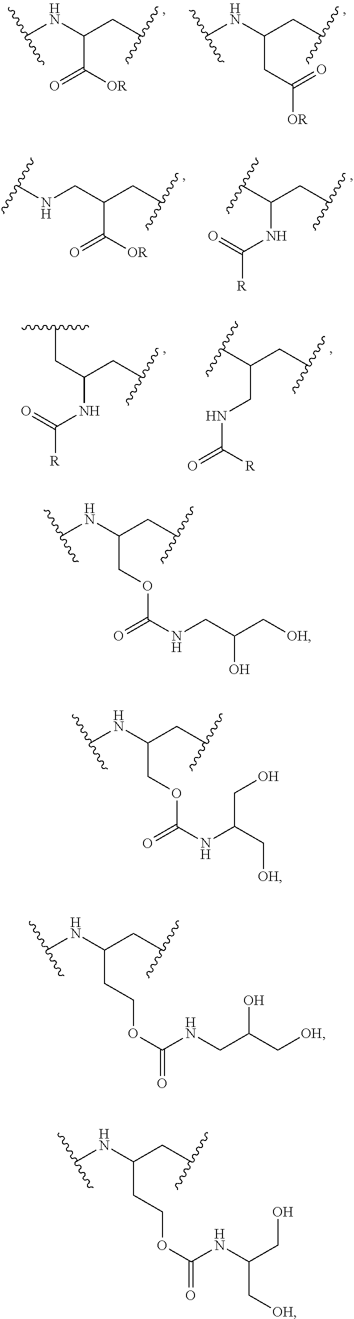

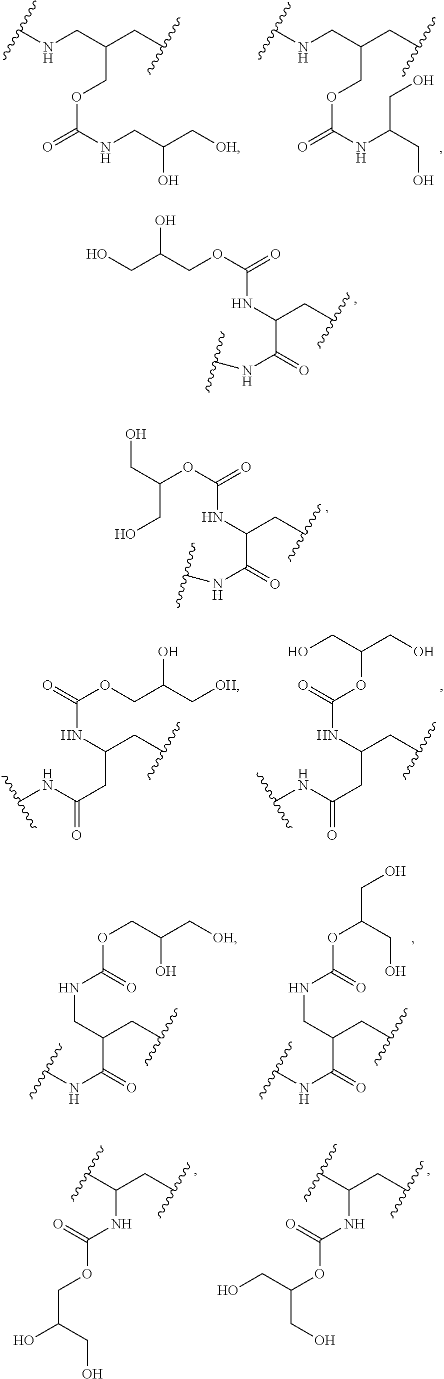

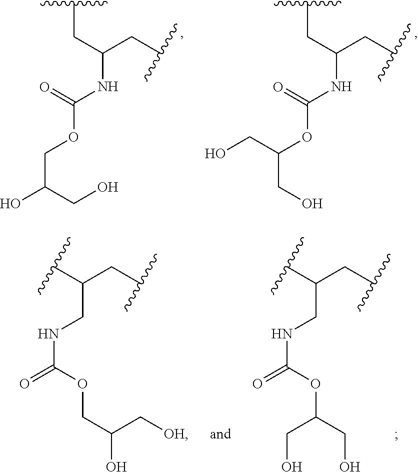 [SMILES: *CC(C*)NC(=O)OCC(O)CO ; *CC(C*)NC(=O)OC(CO)CO ; *CC(*)CNC(=O)OCC(O)CO ; *CC(*)CNC(=O)OC(CO)CO]

wherein the representation of L₁ is limited directionally left to right as is depicted, with the right hand side connected to one of the two S groups shown in Formula II;

R^{I-1}, R^{I-2}, R^{I-3}, and R^{I-5} each represent independently for each occurrence hydrogen or C₁-C₄ alkyl;

R^{I-4} is

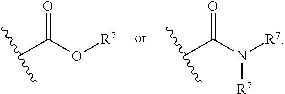

W₁ is independently a bond, O, or N(R^{I-1});

j is 0 or 1;

k and k* is independently 0 or 1;

n* is independently 1, 2, or 3, with the proviso that when n*=1 then W₁ cannot be O or NR^{I-1};

each R is independently —H, —C₁-C₆ alkyl, phenyl, benzyl, —CH₂CO₂R^{I-1}, —CH₂CON(R^{I-1})(R^{I-1});

Y^{I-1} is independently selected from the group consisting of

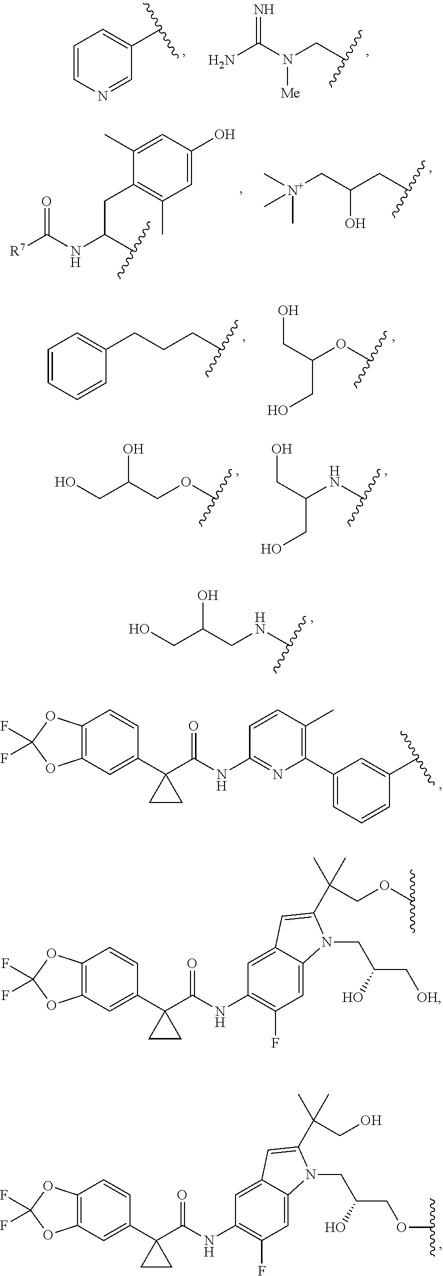

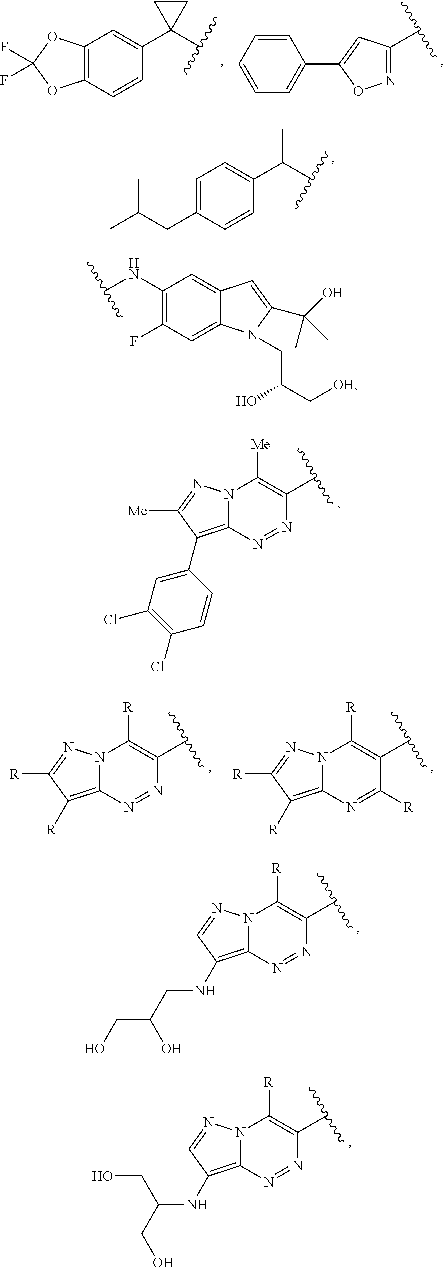

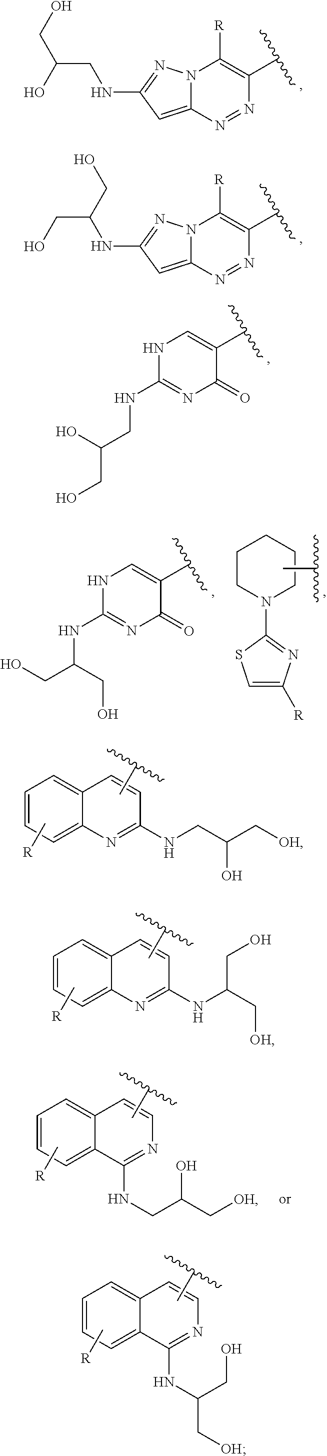

Z* is

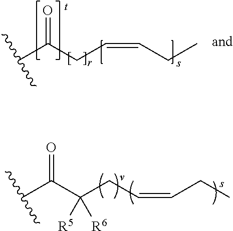

wherein:
R⁵, R⁶ and R⁷ each represent independently for each occurrence hydrogen, C₁-C₄ alkyl, or halogen;
r is 2, 3, or 7;
s is 3, 5, or 6;
t is 0 or 1; and
v is 1, 2, or 6.
XX. A compound of Formula III:

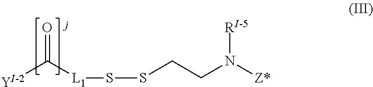

or a pharmaceutically acceptable salt or solvate thereof; wherein:

L₁ is independently selected from the group consisting of

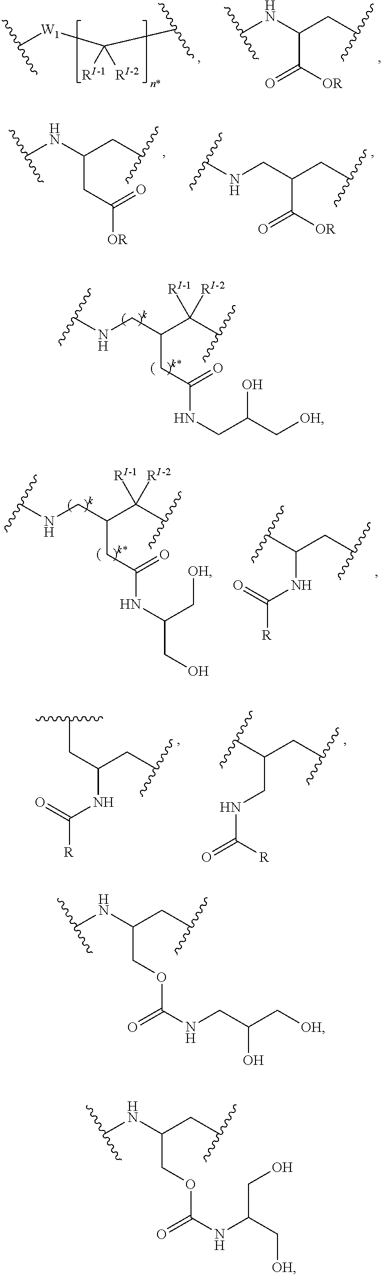

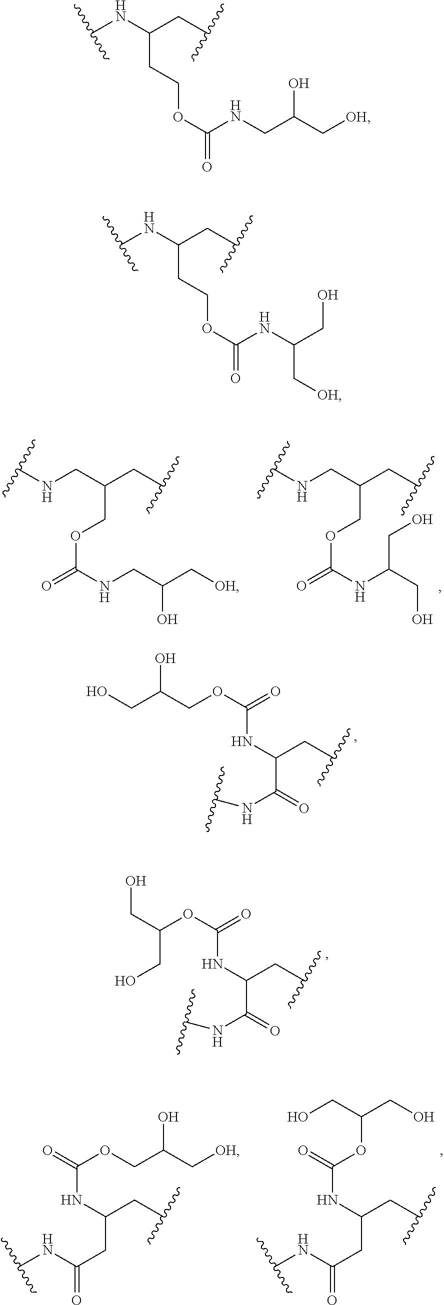

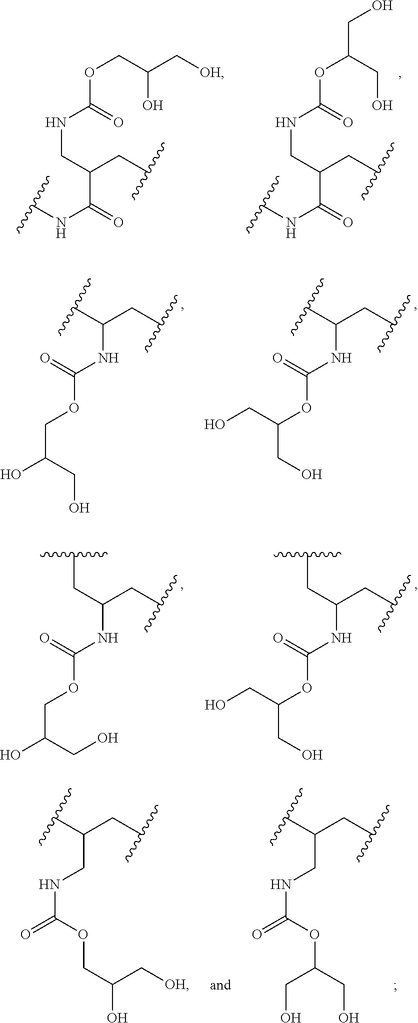

wherein the representation of L₁ is limited directionally left to right as is depicted, with the right hand side connected to one of the two S groups shown in Formula III;

R^{I-1}, R^{I-2}, and R^{I-5} each represent independently for each occurrence hydrogen or C₁-C₄ alkyl;

W₁ is independently a bond, O, or N(R^{I-1});

j is 0 or 1;

n* is independently 1, 2, or 3, with the proviso that when n*=1 then W₁ cannot be O or NR^{I-1};

each R is independently —H, —C₁-C₆ alkyl, phenyl, benzyl, —CH₂CO₂R^{I-1}, —CH₂CON(R^{I-1})(R^{I-1});

Y^{I-2} is independently selected from the group consisting of

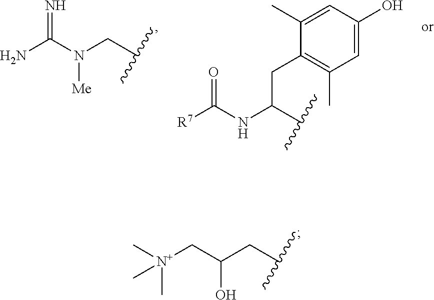

Z* is

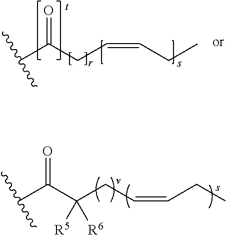

wherein:
R⁵, R⁶, and R⁷ independently are hydrogen, C₁-C₄ alkyl, or halogen;
r is 2, 3, or 7;
s is 3, 5, or 6;
t is 0 or 1; and

v is 1, 2, or 6.
XXI. The compound of embodiment I or XIX, wherein the compound is one of the following compounds or a pharmaceutically acceptable salt thereof:

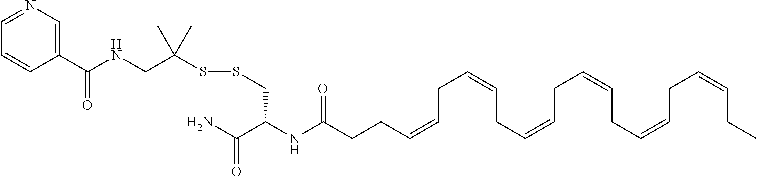 [SMILES: CC/C=C\C/C=C\C/C=C\C/C=C\C/C=C\C/C=C\CCC(=O)N[C@@H](CSSC(C)(C)CNC(=O)c1cccnc1)C(N)=O]

N-(2-(((R)-3-amino-2-((4Z,7Z,10Z,13Z,16Z,19Z)-docosa-4,7,10,13,16,19-hexaenamido)-3-oxopropyl)disulfanyl)-2-methylpropyl)nicotinamide (I-1);

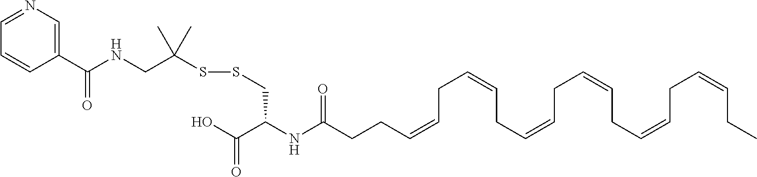 [SMILES: CC/C=C\C/C=C\C/C=C\C/C=C\C/C=C\C/C=C\CCC(=O)N[C@@H](CSSC(C)(C)CNC(=O)c1cccnc1)C(=O)O]

N-((4Z,7Z,10Z,13Z,16Z,19Z)-docosa-4,7,10,13,16,19-hexaenoyl)-S-((2-methyl-1-(nicotinamido)propan-2-yl)thio)-L-cysteine (I-3);

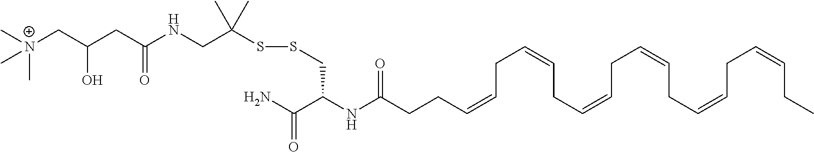 [SMILES: CC/C=C\C/C=C\C/C=C\C/C=C\C/C=C\C/C=C\CCC(=O)N[C@@H](CSSC(C)(C)CNC(=O)CC(O)C[N+](C)(C)C)C(N)=O]

4-((2-(((R)-3-amino-2-((4Z,7Z,10Z,13Z,16Z,19Z)-docosa-4,7,10,13,16,19-hexaenamido)-3-oxopropyl)disulfanyl)-2-methylpropyl)amino)-2-hydroxy-N,N,N-trimethyl-4-oxobutan-1-aminium (I-5);

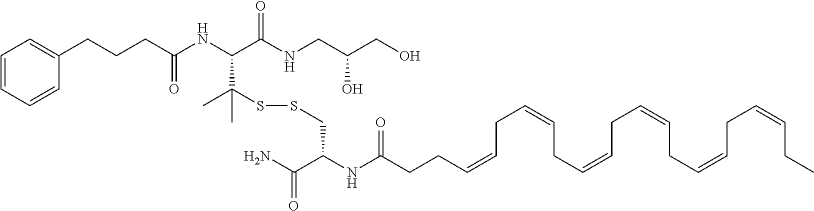 [SMILES: CC/C=C\C/C=C\C/C=C\C/C=C\C/C=C\C/C=C\CCC(=O)N[C@@H](CSSC(C)(C)[C@H](NC(=O)CCCc1ccccc1)C(=O)NC[C@@H](O)CO)C(N)=O]

(4Z,7Z,10Z,13Z,16Z,19Z)—N—((R)-1-amino-3-(((R)-4-(((R)-2,3-dihydroxypropyl)amino)-2-methyl-4-oxo-3-(4-phenylbutanamido)butan-2-yl)disulfanyl)-1-oxopropan-2-yl)docosa-4,7,10,13,16,19-hexaenamide (I-10);

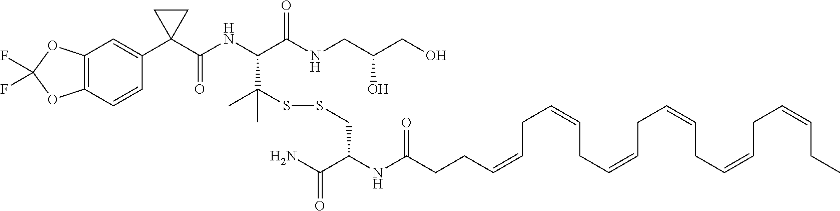 [SMILES: CC/C=C\C/C=C\C/C=C\C/C=C\C/C=C\C/C=C\CCC(=O)N[C@@H](CSSC(C)(C)[C@H](NC(=O)C1(c2ccc3c(c2)OC(F)(F)O3)CC1)C(=O)NC[C@@H](O)CO)C(N)=O]

N—((R)-3-(((R)-3-amino-2-((4Z,7Z,10Z,13Z,16Z,19Z)-docosa-4,7,10,13,16,19-hexaenamido)-3-oxopropyl)disulfanyl)-1-(((R)-2,3-dihydroxypropyl)amino)-3-methyl-1-oxobutan-2-yl)-1-(2,2-difluorobenzo[d][1,3]dioxol-5-yl)cyclopropane-1-carboxamide (I-12); or

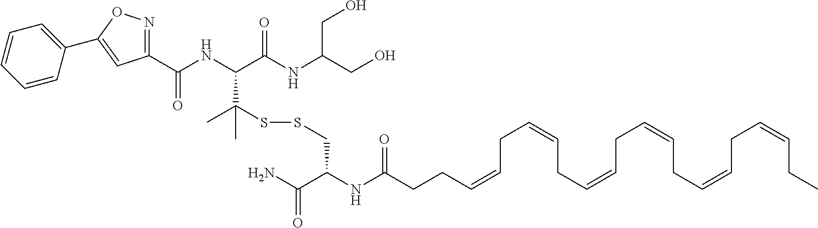 [SMILES: CC/C=C\C/C=C\C/C=C\C/C=C\C/C=C\C/C=C\CCC(=O)N[C@@H](CSSC(C)(C)[C@H](NC(=O)c1cc(-c2ccccc2)on1)C(=O)NC(CO)CO)C(N)=O]

N—((R)-3-(((R)-3-amino-2-((4Z,7Z,10Z,13Z,16Z,19Z)-docosa-4,7,10,13,16,19-hexaenamido)-3-oxopropyl)disulfanyl)-1-((1,3-dihydroxypropan-2-yl)amino)-3-methyl-1-oxobutan-2-yl)-5-phenylisoxazole-3-carboxamide (I-15).
XXII. The compound of embodiment XX, wherein the compound is one of the following compounds or a pharmaceutically acceptable salt thereof:

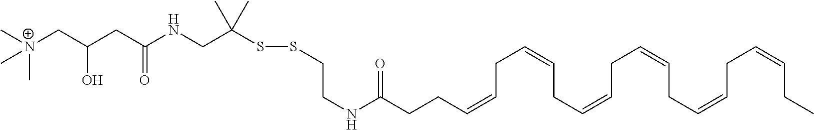 [SMILES: CC/C=C\C/C=C\C/C=C\C/C=C\C/C=C\C/C=C\CCC(=O)NCCSSC(C)(C)CNC(=O)CC(O)C[N+](C)(C)C]

4-((2-((2-((4Z,7Z,10Z,13Z,16Z,19Z)-docosa-4,7,10,13,16,19-hexaenamido)ethyl)disulfanyl)-2-methylpropyl)amino)-2-hydroxy-N,N,N-trimethyl-4-oxobutan-1-aminium (III-1);

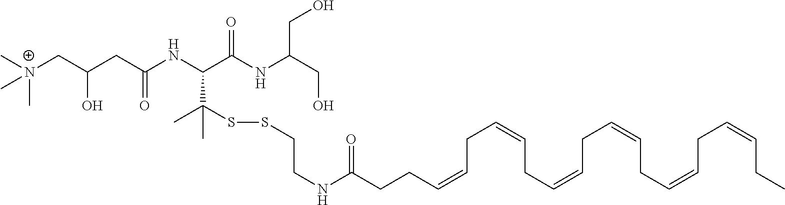 [SMILES: CC/C=C\C/C=C\C/C=C\C/C=C\C/C=C\C/C=C\CCC(=O)NCCSSC(C)(C)[C@H](NC(=O)CC(O)C[N+](C)(C)C)C(=O)NC(CO)CO]

4-(((R)-1-((1,3-dihydroxypropan-2-yl)amino)-3-((2-((4Z,7Z,10Z,13Z,16Z,19Z)-docosa-4,7,10,13,16,19-hexaenamido)ethyl)disulfanyl)-3-methyl-1-oxobutan-2-yl)amino)-2-hydroxy-N,N,N-trimethyl-4-oxobutan-1-aminium (III-2);

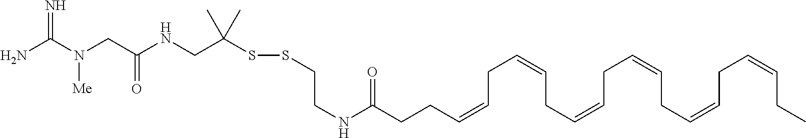 [SMILES: CC/C=C\C/C=C\C/C=C\C/C=C\C/C=C\C/C=C\CCC(=O)NCCSSC(C)(C)CNC(=O)CN(C)C(=N)N]

(4Z,7Z,10Z,13Z,16Z,19Z)—N-(2-((2-methyl-1-(2-(1-methylguanidino)acetamido)propan-2-yl)disulfanyl)ethyl)docosa-4,7,10,13,16,19-hexaenamide (III-4);

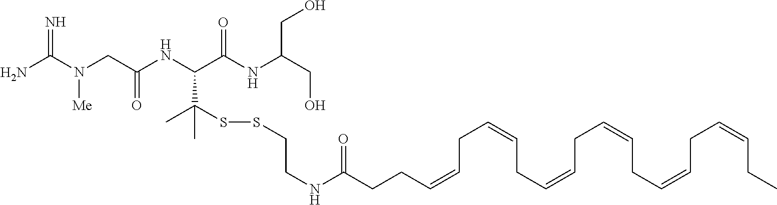 [SMILES: CC/C=C\C/C=C\C/C=C\C/C=C\C/C=C\C/C=C\CCC(=O)NCCSSC(C)(C)[C@H](NC(=O)CN(C)C(=N)N)C(=O)NC(CO)CO]

(4Z,7Z,10Z,13Z,16Z,19Z)—N-(2-(((R)-4-((1,3-dihydroxypropan-2-yl)amino)-2-methyl-3-(2-(1-methylguanidino)acetamido)-4-oxobutan-2-yl)disulfanyl)ethyl)docosa-4,7,10,13,16,19-hexaenamide (III-5); or

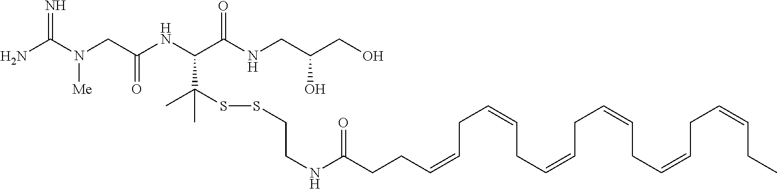 [SMILES: CC/C=C\C/C=C\C/C=C\C/C=C\C/C=C\C/C=C\CCC(=O)NCCSSC(C)(C)[C@H](NC(=O)CN(C)C(=N)N)C(=O)NC[C@@H](O)CO]

(4Z,7Z,10Z,13Z,16Z,19Z)—N-(2-(((R)-4-(((R)-2,3-dihydroxypropyl)amino)-2-methyl-3-(2-(1-methylguanidino)acetamido)-4-oxobutan-2-yl)disulfanyl)ethyl)docosa-4,7,10,13,16,19-hexaenamide (III-6).
XXIII. The compound of embodiment I or XIX, wherein the compound is:

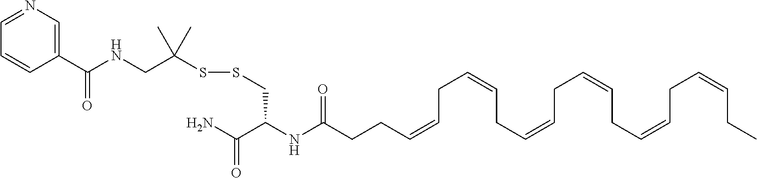 [SMILES: CC/C=C\C/C=C\C/C=C\C/C=C\C/C=C\C/C=C\CCC(=O)N[C@@H](CSSC(C)(C)CNC(=O)c1cccnc1)C(N)=O]

N-(2-(((R)-3-amino-2-((4Z,7Z,10Z,13Z,16Z,19Z)-docosa-4,7,10,13,16,19-hexaenamido)-3-oxopropyl)disulfanyl)-2-methylpropyl)nicotinamide (I-1) or a pharmaceutically acceptable salt thereof.
XXIV. The compound of embodiment I or XIX, wherein the compound is:

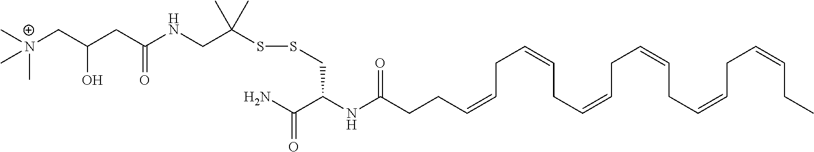 [SMILES: CC/C=C\C/C=C\C/C=C\C/C=C\C/C=C\C/C=C\CCC(=O)N[C@@H](CSSC(C)(C)CNC(=O)CC(O)C[N+](C)(C)C)C(N)=O]

4-((2-(((R)-3-amino-2-((4Z,7Z,10Z,13Z,16Z,19Z)-docosa-4,7,10,13,16,19-hexaenamido)-3-oxopropyl)disulfanyl)-2-methylpropyl)amino)-2-hydroxy-N,N,N-trimethyl-4-oxobutan-1-aminium (I-5) or a pharmaceutically acceptable salt thereof.
XXV. The compound of embodiment I or XIX, wherein the compound is:

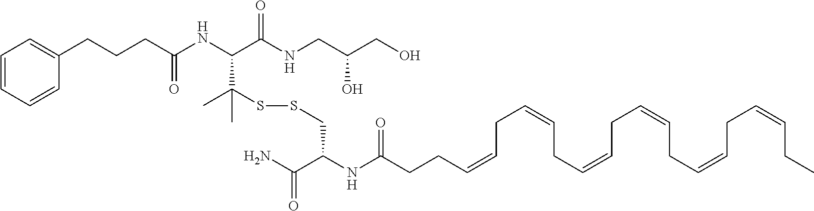 [SMILES: CC/C=C\C/C=C\C/C=C\C/C=C\C/C=C\C/C=C\CCC(=O)N[C@@H](CSSC(C)(C)[C@H](NC(=O)CCCc1ccccc1)C(=O)NC[C@@H](O)CO)C(N)=O]

(4Z,7Z,10Z,13Z,16Z,19Z)—N—((R)-1-amino-3-(((R)-4-(((R)-2,3-dihydroxypropyl)amino)-2-methyl-4-oxo-3-(4-phenylbutanamido)butan-2-yl)disulfanyl)-1-oxopropan-2-yl)docosa-4,7,10,13,16,19-hexaenamide (I-10) or a pharmaceutically acceptable salt thereof.
XXVI. The compound of embodiment I or XIX, wherein the compound is:

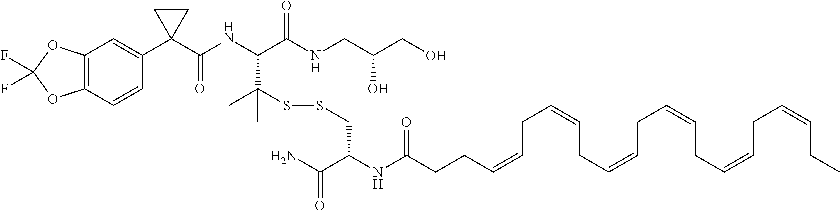 [SMILES: CC/C=C\C/C=C\C/C=C\C/C=C\C/C=C\C/C=C\CCC(=O)N[C@@H](CSSC(C)(C)[C@H](NC(=O)C1(c2ccc3c(c2)OC(F)(F)O3)CC1)C(=O)NC[C@@H](O)CO)C(N)=O]

N—((R)-3-(((R)-3-amino-2-((4Z,7Z,10Z,13Z,16Z,19Z)-docosa-4,7,10,13,16,19-hexaenamido)-3-oxopropyl)disulfanyl)-1-(((R)-2,3-dihydroxypropyl)amino)-3-methyl-1-oxobutan-2-yl)-1-(2,2-difluorobenzo[d][1,3]dioxol-5-yl)cyclopropane-1-carboxamide (I-12) or a pharmaceutically acceptable salt thereof.
XXVII. The compound of embodiment XX, wherein the compound is:

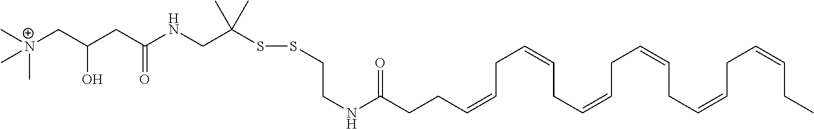 [SMILES: CC/C=C\C/C=C\C/C=C\C/C=C\C/C=C\C/C=C\CCC(=O)NCCSSC(C)(C)CNC(=O)CC(O)C[N+](C)(C)C]

4-((2-((2-((4Z,7Z,10Z,13Z,16Z,19Z)-docosa-4,7,10,13,16,19-hexaenamido)ethyl)disulfanyl)-2-methylpropyl)amino)-2-hydroxy-N,N,N-trimethyl-4-oxobutan-1-aminium (III-1) or a pharmaceutically acceptable salt thereof.
XXVIII. The compound of embodiment XX, wherein the compound is:

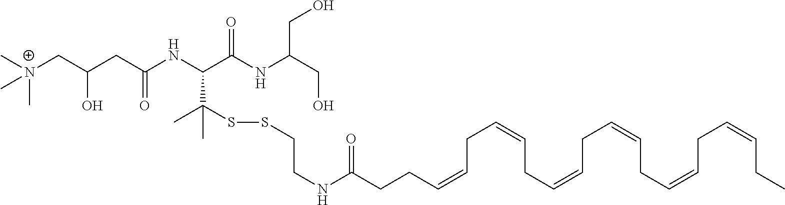 [SMILES: CC/C=C\C/C=C\C/C=C\C/C=C\C/C=C\C/C=C\CCC(=O)NCCSSC(C)(C)[C@H](NC(=O)CC(O)C[N+](C)(C)C)C(=O)NC(CO)CO]

4-(((R)-1-((1,3-dihydroxypropan-2-yl)amino)-3-((2-((4Z,7Z,10Z,13Z,16Z,19Z)-docosa-4,7,10,13,16,19-hexaenamido)ethyl)disulfanyl)-3-methyl-1-oxobutan-2-yl)amino)-2-hydroxy-N,N,N-trimethyl-4-oxobutan-1-aminium (III-2) or a pharmaceutically acceptable salt thereof.
XXIX. The compound of embodiment XX, wherein the compound is:

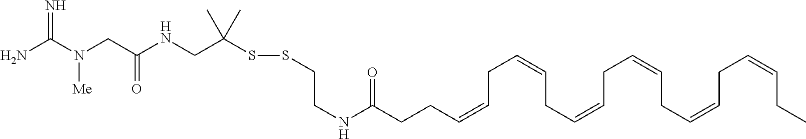 [SMILES: CC/C=C\C/C=C\C/C=C\C/C=C\C/C=C\C/C=C\CCC(=O)NCCSSC(C)(C)CNC(=O)CN(C)C(=N)N]

(4Z,7Z,10Z,13Z,16Z,19Z)—N-(2-((2-methyl-1-(2-(1-methylguanidino)acetamido)propan-2-yl)disulfanyl)ethyl)docosa-4,7,10,13,16,19-hexaenamide (III-4) or a pharmaceutically acceptable salt thereof.
XXX. The compound of embodiment XX, wherein the compound is

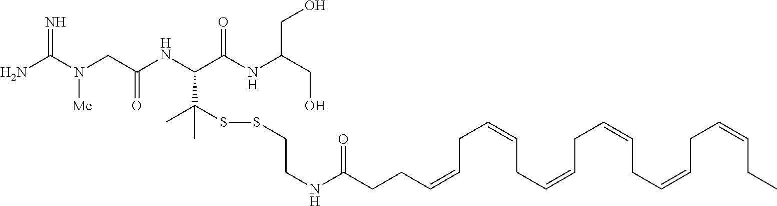 [SMILES: CC/C=C\C/C=C\C/C=C\C/C=C\C/C=C\C/C=C\CCC(=O)NCCSSC(C)(C)[C@H](NC(=O)CN(C)C(=N)N)C(=O)NC(CO)CO]

(4Z,7Z,10Z,13Z,16Z,19Z)—N-(2-(((R)-4-((1,3-dihydroxypropan-2-yl)amino)-2-methyl-3-(2-(1-methylguanidino)acetamido)-4-oxobutan-2-yl)disulfanyl)ethyl)docosa-4,7,10,13,16,19-hexaenamide (III-5) or a pharmaceutically acceptable salt thereof.
XXXI. A compound of Formula IV

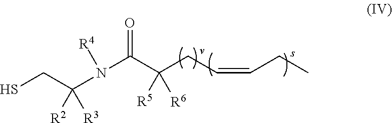

or a pharmaceutically acceptable salt or solvate thereof; wherein:

R² and R⁴ each represent independently for each occurrence hydrogen or C₁-C₄ alkyl;

R³ is

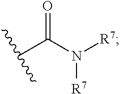

R⁵, R⁶, and R⁷ are independently hydrogen, C₁-C₄ alkyl, or halogen;

s is 3, 5, or 6; and

v is 1, 2, or 6.
XXXII. The compound of embodiment XXXI, wherein the compound is one of the following or a pharmaceutically acceptable salt thereof:

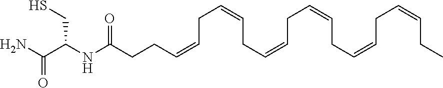 [SMILES: CC/C=C\C/C=C\C/C=C\C/C=C\C/C=C\C/C=C\CCC(=O)N[C@@H](CS)C(N)=O]

(4Z,7Z,10Z,13Z,16Z,19Z)—N—((R)-1-amino-3-mercapto-1-oxopropan-2-yl)docosa-4,7,10,13,16,19-hexaenamide (IV-1); or

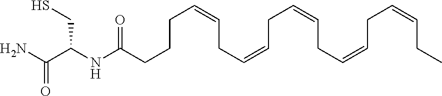 [SMILES: CC/C=C\C/C=C\C/C=C\C/C=C\C/C=C\CCCC(=O)N[C@@H](CS)C(N)=O]

(5Z,8Z,11Z,14Z,17Z)—N—((R)-1-amino-3-mercapto-1-oxopropan-2-yl)icosa-5,8,11,14,17-pentaenamide (IV-3).
XXXIII. A pharmaceutical composition comprising a compound of any one of embodiments I-XXXII and a pharmaceutically acceptable carrier.
XXXIV. A method of treating a disease selected from the group consisting of CF, a neurodegenerative disease, inflammatory disease, liver disease, muscle disease, infection, and immune disease in a patient, the method comprising administering to a subject in need thereof a therapeutically effective amount of the compound of any one of embodiments I-XXXII or the pharmaceutical composition of embodiment XXXIII to treat the disease.
XXXV. The method of embodiment XXXIV, wherein the disease is CF.
XXXVI. The method of embodiment XXXIV, wherein the disease is a neurodegenerative disease.
XXXVII. The method of embodiment XXXVI, wherein the neurodegenerative disease is Huntington's disease, Alzheimer's disease, or Parkinson's disease.
XXXVIII. A method of embodiment XXXIV, wherein the method further comprises administering a second therapeutic agent selected from the group consisting of Ivacaftor (VX-770), Lumacaftor (VX-809), VX-661, Orkambi (a combination of VX-770 and VX-809), and a combination of VX-661 and VX-770.
XXXIX. The method of embodiment XXXIV, wherein the disease is idiopathic pulmonary fibrosis.
XL. A method of activating autophagy in a subject, the method comprising administering to a subject in need thereof an effective amount of the compound of any one of embodiments I-XXXII or the pharmaceutical composition of embodiment XXXIII to activate autophagy in the subject.
XLI. The method of embodiment XL, wherein the administering increases the ratio of LC3-II to LC3-I in the subject by at least 10%.
XLII. The method of embodiment XL or XLI, wherein the administering decreases the amount of p62 protein in the subject by at least 1% w/w.
XLIII. The method of any one of embodiments XXXIV-XLII, wherein the patient has been diagnosed as having CF or a neurodegenerative disease or idiopathic pulmonary fibrosis.
XLIV. The method of any one of embodiments XXXIV-XLIII, wherein the patient is a human.

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A compound of Formula I: 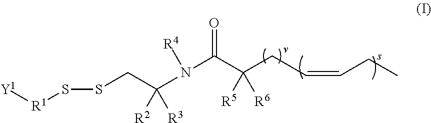
or a pharmaceutically acceptable salt or solvate thereof; wherein:
R¹ is C₁-C₆ alkylene optionally substituted by —C(O)N(R²)(hydroxyalkyl), —N(R²)C(O)-(hydroxyalkyl), —O-(hydroxyalkyl), —N(R²)-(hydroxyalkyl), —OC(O)N(R²)(hydroxyalkyl), —C(O)N(R²)(R⁸), —N(R²)C(O)R⁹, or —CO₂R⁹;
R³ is 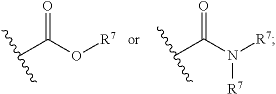
R², R⁴, and R⁷ each represent independently for each occurrence hydrogen or C₁-C₄ alkyl;
R⁵ and R⁶ are independently hydrogen, C₁-C₄ alkyl, or halogen;
R⁸ is C₁-C₆ alkyl, phenyl, benzyl, C₁-C₆ alkylene-CO₂R⁷, or C₁-C₆ alkylene-C(O)N(R⁷)₂;
R⁹ represents independently for each occurrence hydrogen, C₁-C₆ alkyl, phenyl, benzyl, C₁-C₆ alkylene-CO₂R⁷, or C₁-C₆ alkylene-C(O)N(R⁷)₂;
s is 3, 5, or 6;
v is 1, 2, or 6;
Y¹ is an amide or carbamate selected from the group consisting of —N(R⁷)C(O)-aralkyl, —N(R⁷)C(O)-(hydroxyalkyl), —C(O)N(R⁷)(hydroxyalkyl), —N(R⁷)CO₂-(hydroxyalkyl), —OC(O)N(R⁷)(hydroxyalkyl), —N(R⁷)C(O)—Z¹, and 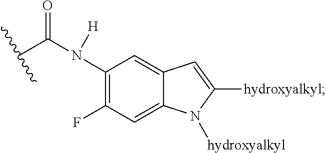
and
Z¹ is one of the following:

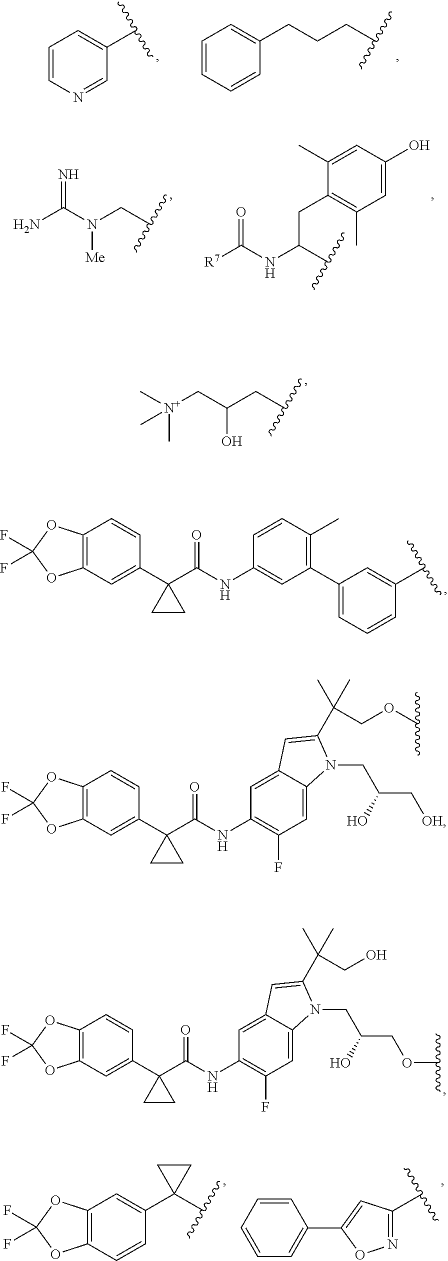

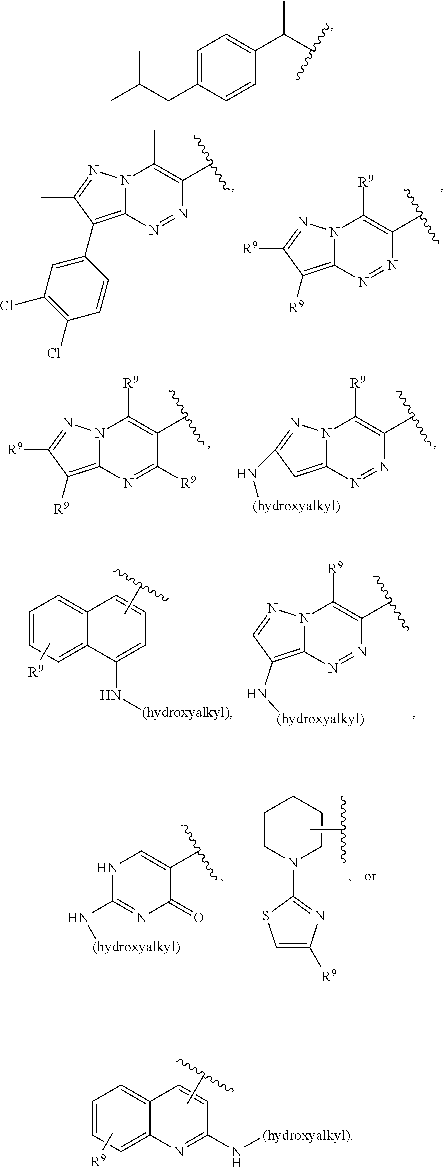

2. The compound of claim 1, wherein R¹ is C₁-C₆ alkylene optionally substituted with —C(O)N(R²)(hydroxyalkyl), —N(R²)C(O)(hydroxyalkyl), —O-(hydroxyalkyl), —N(R²)-(hydroxyalkyl) or —OC(O)N(R²)(hydroxyalkyl).

3. The compound of claim 1, wherein R¹ is C₁-C₆ alkylene.

4. The compound of claim 3, wherein R¹ is ethylene.

5. The compound of claim 1, wherein R¹ is C₂-C₄ alkylene optionally substituted with one of the following:

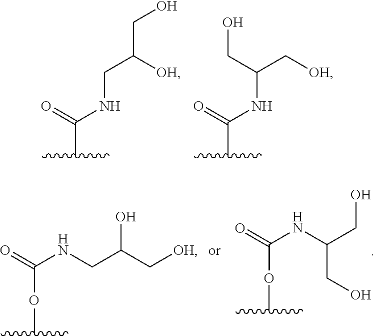 [SMILES: *C(=O)NCC(O)CO ; *C(=O)NC(CO)CO ; *OC(=O)NCC(O)CO ; *OC(=O)NC(CO)CO]

6. The compound of claim 1, wherein R² and R⁴ are hydrogen.

7. The compound of claim 1, wherein R⁵ and R⁶ are hydrogen.

8. The compound of claim 1, wherein R⁷ is hydrogen.

9. The compound of claim 1, wherein R³ is

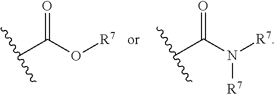

10. The compound of claim 1, wherein v is 1, and s is 6.

11. The compound of claim 1, wherein v is 2, and s is 5.

12. The compound of claim 1, wherein Y¹ is —N(R⁷)C(O)-aralkyl.

13. The compound of claim 1, wherein Y¹ is —N(R⁷)C(O)—(CH₂)₃-phenyl.

14. The compound of claim 1, wherein Y¹ is —N(R⁷)C(O)-(hydroxyalkyl), —C(O)N(R⁷)(hydroxyalkyl), —N(R⁷)CO₂-(hydroxyalkyl), or —OC(O)N(R⁷)(hydroxyalkyl).

15. The compound of claim 1, wherein Y¹ is selected from:

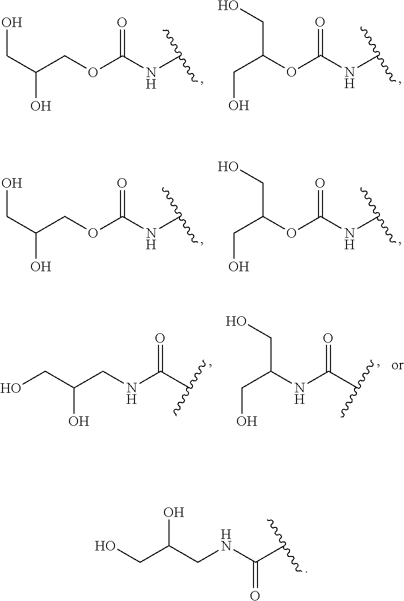

16. The compound of claim 1, wherein Y¹ is —N(R⁷)C(O)—Z¹.

17. The compound of claim 1, wherein Z¹ is selected from the group consisting of

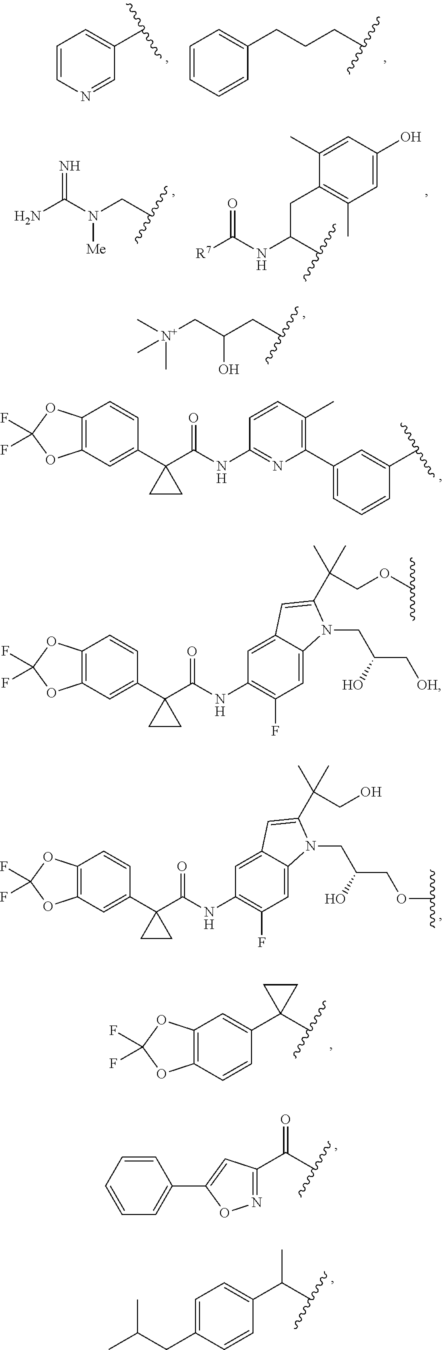

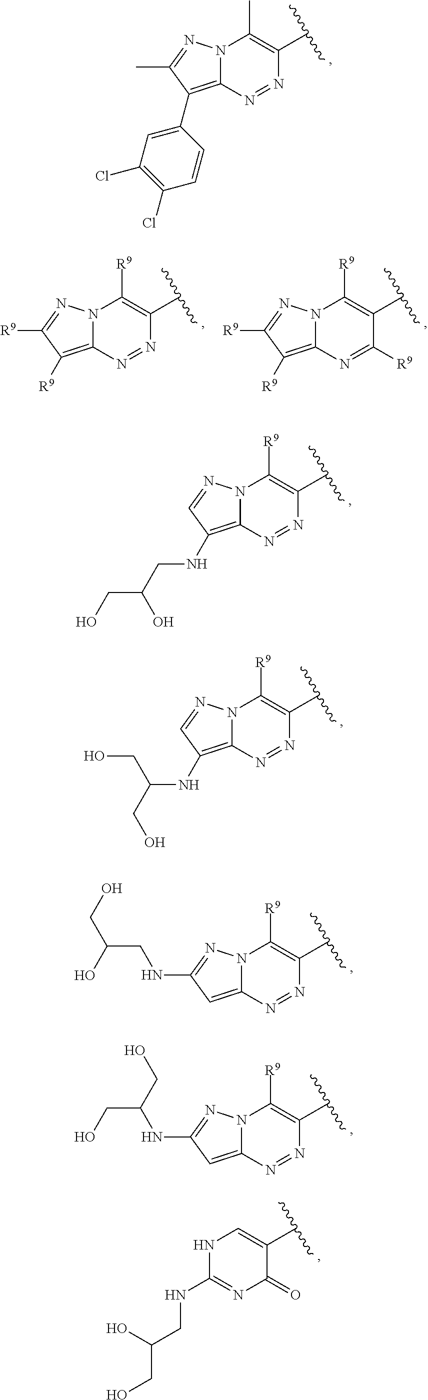

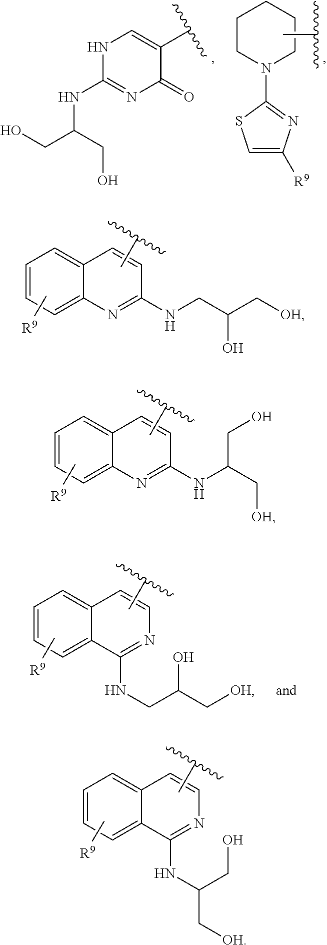

18. The compound of claim 1, wherein Y¹ is

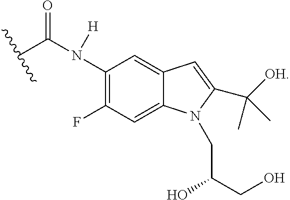 [SMILES: *C(=O)Nc1cc2cc(C(C)(C)O)n(C[C@@H](O)CO)c2cc1F]

19. A compound of Formula II: 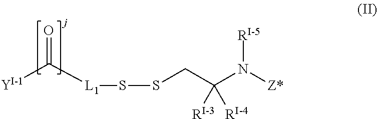
or a pharmaceutically acceptable salt or solvate thereof; wherein:
L₁ is independently selected from the group consisting of 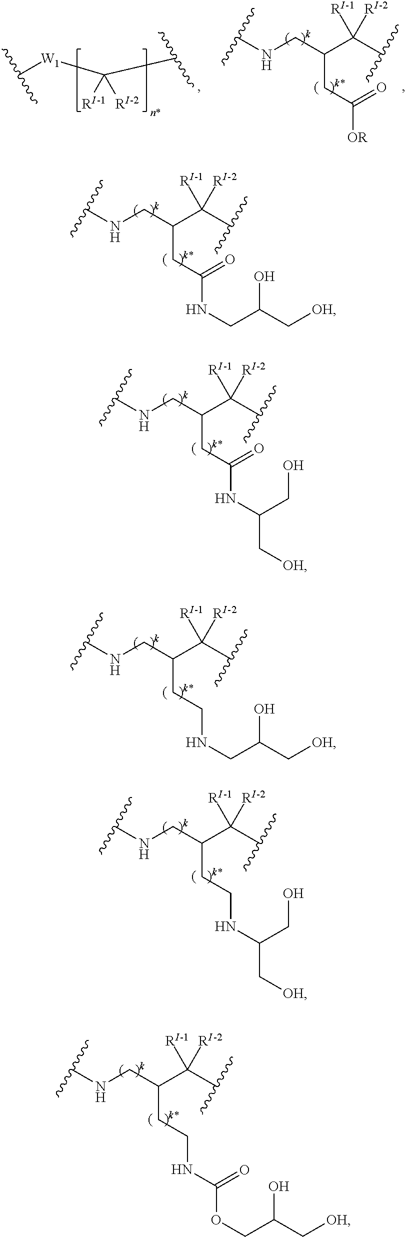 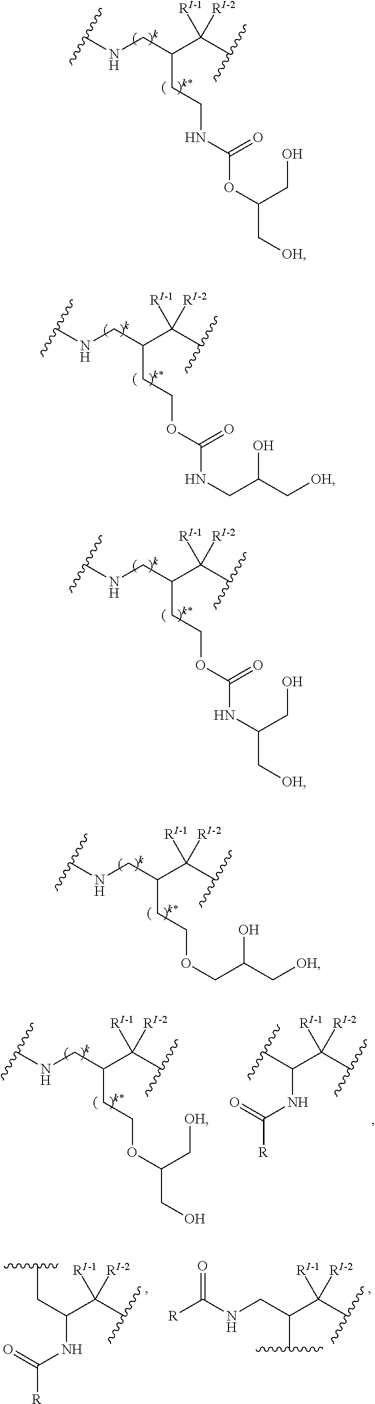 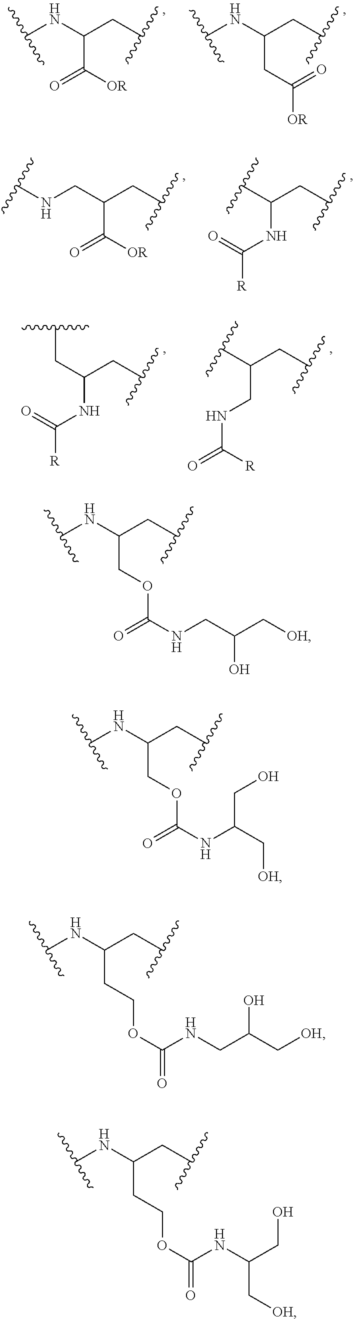 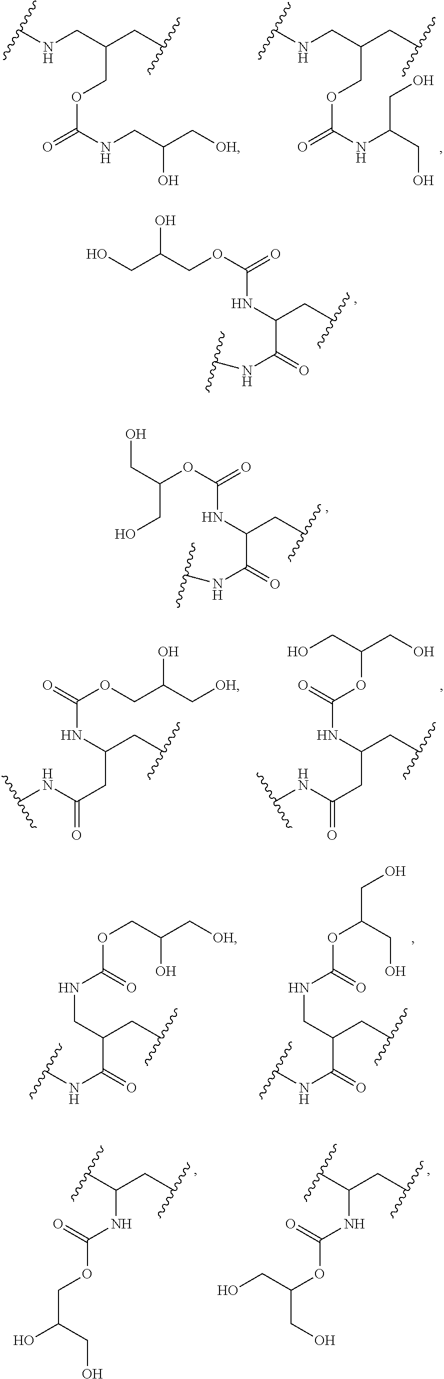 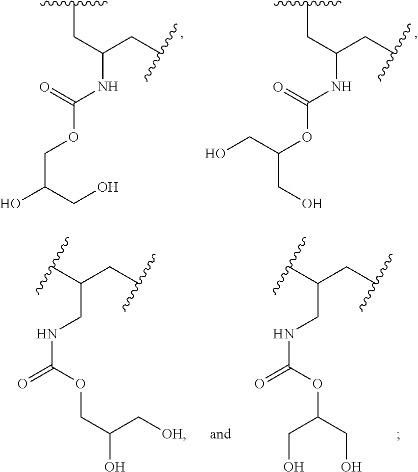 [SMILES: *CC(C*)NC(=O)OCC(O)CO ; *CC(C*)NC(=O)OC(CO)CO ; *CC(*)CNC(=O)OCC(O)CO ; *CC(*)CNC(=O)OC(CO)CO]
wherein the representation of L₁ is limited directionally left to right as is depicted, with the right hand side connected to one of the two S groups shown in Formula II;
R^{I-1}, R^{I-2}, R^{I-1}, and R^{I-5} each represent independently for each occurrence hydrogen or C₁-C₄ alkyl;
R^{I-4} is 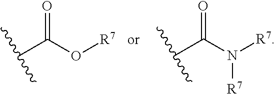
W₁ is independently a bond, O, or N(R^{I-1});
j is 0 or 1;
k and k* is independently 0 or 1;
n* is independently 1, 2, or 3, with the proviso that when n*=1 then W₁ cannot be O or NR^{I-1};
each R is independently —H, —C₁-C₆ alkyl, phenyl, benzyl, —CH₂CO₂R^{I-1}, —CH₂CON(R^{I-1})(R^{I-1});
Y^{I-1} is independently selected from the group consisting of 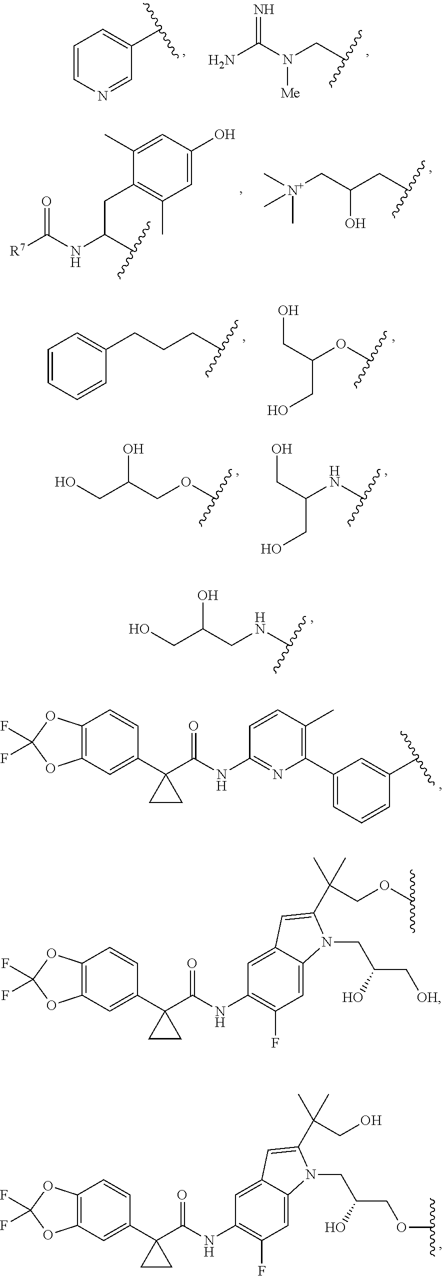 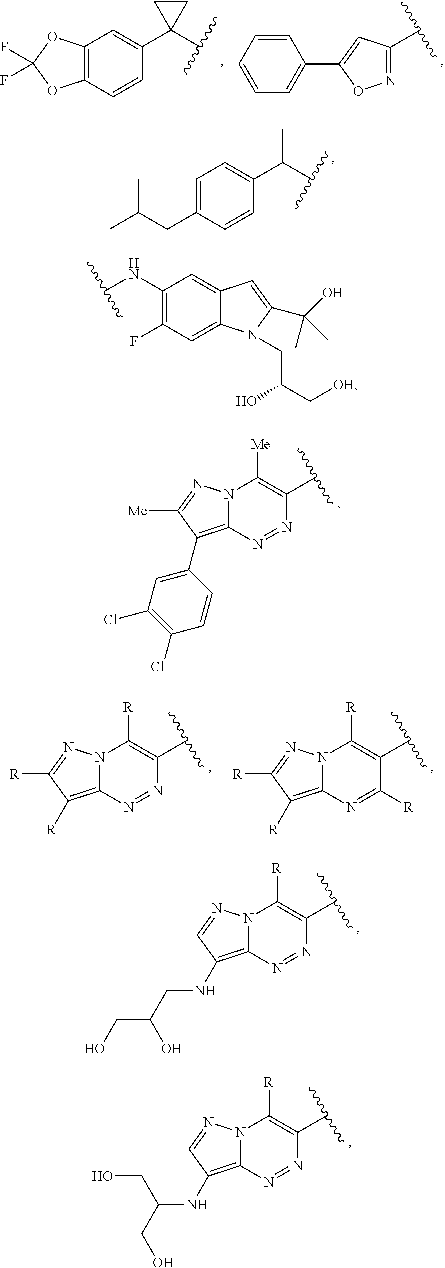 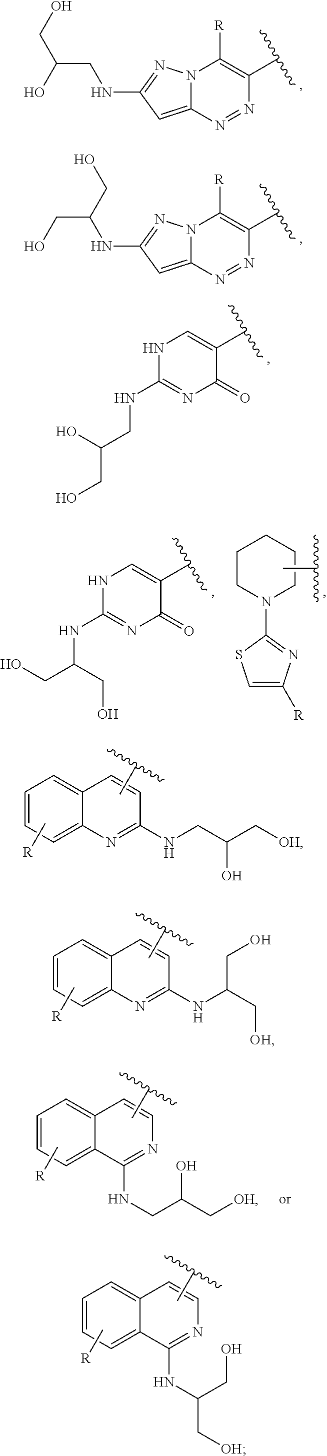
Z* is 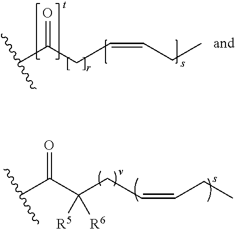
wherein:
R⁵, R⁶ and R⁷ each represent independently for each occurrence hydrogen, C₁-C₄ alkyl, or halogen;
r is 2, 3, or 7;
s is 3, 5, or 6;
t is 0 or 1; and
v is 1, 2, or 6.

20. A compound of Formula III: 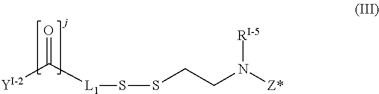
or a pharmaceutically acceptable salt or solvate thereof; wherein:
L₁ is independently selected from the group consisting of 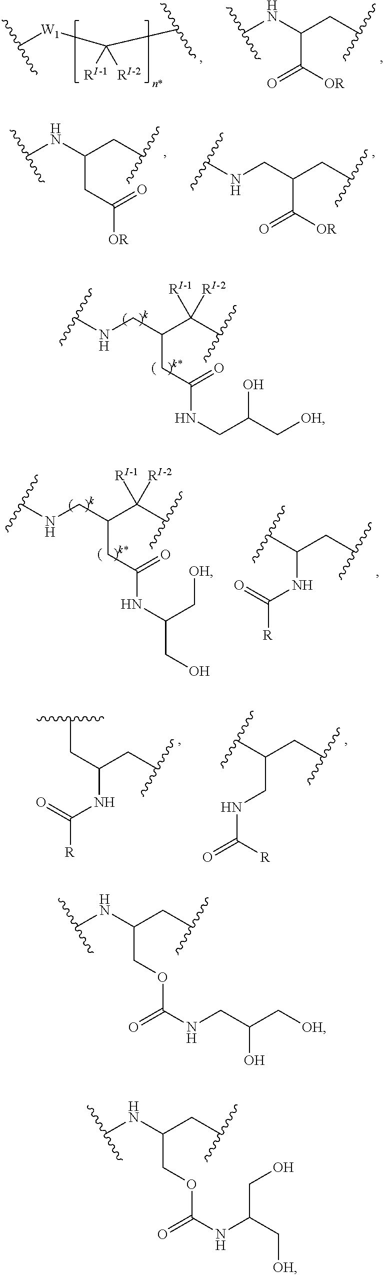 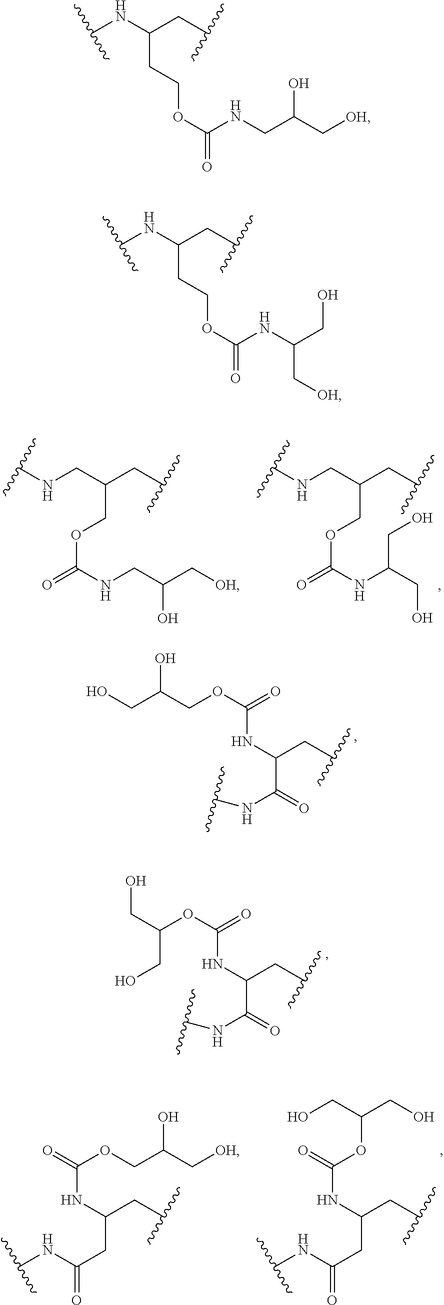 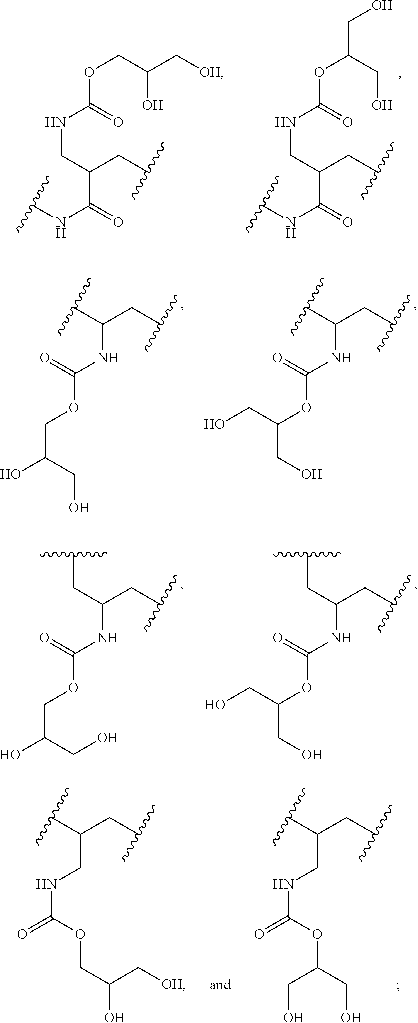
wherein the representation of L₁ is limited directionally left to right as is depicted, with the right hand side connected to one of the two S groups shown in Formula III;
R^{I-1}, R^{I-2}, and R^{I-5} each represent independently for each occurrence hydrogen or C₁-C₄ alkyl;
W₁ is independently a bond, O, or N(R^{I-1});
j is 0 or 1;
n* is independently 1, 2, or 3, with the proviso that when n*=1 then W₁ cannot be O or NR^{I-1};
each R is independently —H, —C₁-C₆ alkyl, phenyl, benzyl, —CH₂CO₂R^{I-1}, —CH₂CON(R^{I-1})(R^{I-1});
Y^{I-2} is independently selected from the group consisting of 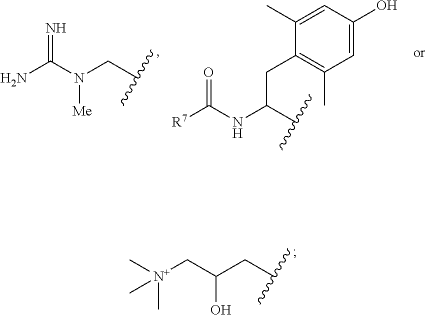
Z* is 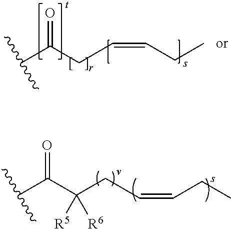
wherein:
R⁵, R⁶, and R⁷ independently are hydrogen, C₁-C₄ alkyl, or halogen;
r is 2, 3, or 7;
s is 3, 5, or 6;
t is 0 or 1; and
v is 1, 2, or 6.

21-44. (canceled)
